(19) United States
(12) Patent Application Publication
(10) Pub. No.: US 2022/0017911 A1
(43) Pub. Date: Jan. 20, 2022

(54) METHODS, COMPOSITIONS, AND MEDIA FOR IMPROVING PLANT TRAITS

(71) Applicant: Pivot Bio, Inc., Berkeley, CA (US)

(72) Inventors: Karsten Temme, Berkeley, CA (US); Alvin Tamsir, Berkeley, CA (US); Sarah Bloch, Berkeley, CA (US); Neal Shah, Berkeley, CA (US); Shayin Gottlieb, Berkeley, CA (US); Richard Broglie, Berkeley, CA (US); Emily Tung, Berkeley, CA (US); Rosemary Clark, Berkeley, CA (US)

(21) Appl. No.: 17/312,172

(22) PCT Filed: Dec. 20, 2019
(86) PCT No.: PCT/US2019/068152

§ 371 Date: Jun. 9, 2021

Related U.S. Application Data
(60) Provisional application No. 62/784,277, filed on Dec. 21, 2018.

(51) Int. Cl. C12N 15/74; C12N 1/20; C12Q 1/04

(52) U.S. Cl. CPC C12N 15/743; C12N 2513/00; C12Q 1/04; C12N 1/20

(57) ABSTRACT

Methods of predicting in planta phenotypes of microbial strains are provided. The methods can include culturing microbial strains in a plant exudate medium and assaying in vitro phenotypes of the microbial strains. The methods may also include using the in vitro phenotypes to predict in planta phenotypes of the microbial strains. Methods of using the microbial strains in field trials are also provided.

### CROSS REFERENCE TO RELATED APPLICATIONS

This application claims the benefit of U.S. Provisional Patent Application No. 62/784,277, filed Dec. 21, 2018, the contents of which is hereby incorporated in its entirety.

### BACKGROUND

Plants are linked to the microbiome via a shared metabolome. A multidimensional relationship between a particular crop trait and the underlying metabolome is characterized by a landscape with numerous local maxima. Optimizing from an inferior local maximum to another representing a better trait by altering the influence of the microbiome on the metabolome may be desirable for a variety of reasons, such as for crop optimization. Economically, environmentally, and socially sustainable approaches to agriculture and food production are required to meet the needs of a growing global population. By 2050 the United Nations' Food and Agriculture Organization projects that total food production must increase by 70% to meet the needs of the growing population, a challenge that is exacerbated by numerous factors, including diminishing freshwater resources, increasing competition for arable land, rising energy prices, increasing input costs, and the likely need for crops to adapt to the pressures of a drier, hotter, and more extreme global climate.

One area of interest is in the improvement of nitrogen fixation. Nitrogen gas (N2) is a major component of the atmosphere of Earth. In addition, elemental nitrogen (N) is an important component of many chemical compounds which make up living organisms. However, many organisms cannot use N₂ directly to synthesize the chemicals used in physiological processes, such as growth and reproduction. In order to utilize the N₂, the N₂ must be combined with hydrogen. The combining of hydrogen with N₂ is referred to as nitrogen fixation. Nitrogen fixation, whether accomplished chemically or biologically, requires an investment of large amounts of energy. In biological systems, an enzyme known as nitrogenase catalyzes the reaction which results in nitrogen fixation. An important goal of nitrogen fixation research is the extension of this phenotype to non-leguminous plants, particularly to important agronomic grasses such as wheat, rice, and maize. Despite enormous progress in understanding the development of the nitrogen-fixing symbiosis between rhizobia and legumes, the path to use that knowledge to induce nitrogen- fixing nodules on non-leguminous crops is still not clear. Meanwhile, the challenge of providing sufficient supplemental sources of nitrogen, such as in fertilizer, will continue to increase with the growing need for increased food production.

### SUMMARY

In some embodiments, the present disclosure provides a method of predicting an in planta phenotype of a microbial strain, the method comprising culturing a microbial strain in a plant exudate medium (PEM); assaying an in vitro phenotype of the microbial strain; and using the in vitro phenotype to predict an in planta phenotype of the microbial strain. In some cases, the microbial strain is isolated from a soil sample. In some cases, the microbial strain is a genetically modified microbial strain. In some cases, the genetically modified microbial strain is produced by random mutagenesis. In some cases, the genetically modified microbial strain is produced by transposon mutagenesis. In some cases, the genetically modified microbial strain is produced by site-directed mutagenesis. In some cases, the genetically modified microbial strain is an endophyte. In some cases, the genetically modified microbial strain is an epiphyte. In some cases, the genetically modified microbial strain is rhizospheric. In some cases, the predicted in planta phenotype is an improved phenotype. In some cases, the predicted in planta phenotype is a worsened phenotype. In some cases, the in vitro phenotype is nitrogen fixation activity. In some cases, nitrogen fixation activity is assayed using an acetylene reduction assay. In some cases, the in vitro phenotype is ammonium excretion. In some cases, the in planta phenotype is plant colonization ability. In some cases, the in vitro phenotype is a growth rate. In some cases, the in vitro phenotype is the peak optical density of a microbial strain culture.

In some cases, the in planta phenotype is rhizosphere fitness. In some cases, rhizosphere fitness is assayed using a colonization assay. In some cases, the colonization assay is conducted in planta in a hydroponic system. In some cases, the colonization assay is conducted in planta in a growth chamber. In some cases, the colonization assay is conducted in planta in a greenhouse. In some cases, the colonization assay is conducted in planta in a field. In some cases, rhizosphere fitness is assayed using a cell growth competition assay. In some cases, the in vitro phenotype is growth in a cell growth competition assay. In some cases, the cell growth competition assay is conducted in a cell culture plate. In some cases, the cell growth competition assay is conducted in a flask. In some cases, the cell growth competition assay is conducted in planta in a hydroponic system. In some cases, the cell growth competition assay is conducted in planta in a grow room or greenhouse. In some cases, the cell growth competition assay is conducted in planta in a field. In some cases, rhizosphere fitness is assayed using two or more cell growth competition assays. In some cases, the two or more cell growth competition assays are conducted under different environmental conditions.

In some embodiments, the present disclosure provides a method of selecting a genetically modified microbial strain having an altered in planta phenotype, the method comprising: culturing a genetically modified microbial strain in a plant exudate medium (PEM); assaying an in vitro phenotype of the genetically modified microbial strain; and selecting the genetically modified microbial strain if it exhibits an alteration in the in vitro phenotype compared to a non-genetically modified microbial strain of the same species cultured under similar conditions, thereby selecting the genetically modified microbial strain having the altered in planta phenotype. In some cases, the altered in planta phenotype is an improved phenotype. In some cases, the altered in planta phenotype is a worsened phenotype. In some cases, the method further comprises introducing a genetic variation into a parent microbial strain to produce the genetically modified microbial strain. In some cases, the parent microbial strain is a non-genetically modified microbial strain of the same species as the genetically modified microbial strain. In some cases, the method further comprises culturing the parent microbial strain in the PEM. In some cases, the in vitro phenotype is nitrogen fixation activity. In some cases, nitrogen fixation activity is assayed using an acetylene reduction assay. In some cases, the in vitro phenotype is ammonium excretion. In some cases, the in planta phenotype is promotion of plant growth. In some cases, the in planta phenotype is plant colonization ability. In some cases, the in vitro phenotype is a growth rate. In some cases, the in vitro phenotype is the peak optical density of a microbial strain culture. In some cases, the in planta phenotype is rhizosphere fitness. In some cases, rhizosphere fitness is assayed using a colonization assay. In some cases, the colonization assay is conducted in planta in a hydroponic system. In some cases, the colonization assay is conducted in planta in a growth chamber. In some cases, the colonization assay is conducted in planta in a greenhouse.

In some cases, the colonization assay is conducted in planta in a field. In some cases, rhizosphere fitness is assayed using a cell growth competition assay. In some cases, the in vitro phenotype is growth in a cell growth competition assay. In some cases, the cell growth competition assay is conducted in a cell culture plate. In some cases, the cell growth competition assay is conducted in a flask. In some cases, the cell growth competition assay is conducted in planta in a hydroponic system. In some cases, the cell growth competition assay is conducted in planta in a grow room or greenhouse. In some cases, the cell growth competition assay is conducted in planta in a field. In some cases, rhizosphere fitness is assayed using two or more cell growth competition assays. In some cases, the two or more cell growth competition assays are conducted under different environmental conditions. In some cases, the genetically modified microbial strain is produced by random mutagenesis. In some cases, the genetically modified microbial strain is produced by transposon mutagenesis. In some cases, the genetically modified microbial strain is produced by site-directed mutagenesis. In some cases, the genetically modified microbial strain is an endophyte.

In some cases, the genetically modified microbial strain is an epiphyte. In some cases, the genetically modified microbial strain is rhizospheric. In some cases, the in planta phenotype is observed when the genetically modified microbial strain is grown with a plant. In some cases, the plant is a cereal plant. In some cases, the plant is selected from the group consisting of: corn, soybean, canola, sorghum, potato, rice, barley, fonio, oats, Palmer's grass, rye, pearl millet, sorghum, spelt, teff, triticale, wheat, breadnut, buckwheat, cattail, chia, flax, grain amaranth, hanza, quinoa, and sesame. In some cases, the plant is selected from the group consisting of: corn, wheat, and rice.

In some embodiments, the present disclosure provides a method of selecting a plant-associated microbe that is attracted to a component of a plant exudate medium (PEM), the method comprising obtaining or providing a semisolid agar plate comprising multiple regions, the multiple regions comprising: a first region comprising agar dissolved in a rich medium; a last region comprising agar dissolved in the PEM; and a plurality of intermediate regions that each comprise a mix of the rich medium and the PEM to form a gradient from said first region to said last region; applying a plurality of putative plant-associated microbes to said first region; culturing the plurality of putative plant-associated microbes for a period of time; and collecting one or more microbes which have migrated the furthest from the first region toward the last region, thereby selecting the plant-associated microbe.

In some cases, the method further comprises selecting a plurality of collected plant-associated microbes above and obtaining an additional semisolid agar plate as described; applying the plurality of collected plant-associated microbes from step to the additional semisolid agar plate; and collecting one or more microbes which have migrated the furthest from the first region toward the last region. In some cases, the plurality of putative plant-associated microbes and/or the plurality of collected plant-associated microbes is cultured for at least 6 hours. In some cases, the plurality of putative plant-associated microbes and/or the plurality of collected plant-associated microbes is cultured for at least 16 hours. In some cases, the plurality of putative plant-associated microbes and/or the plurality of collected plant-associated microbes is cultured for at least one day. In some cases, the plurality of putative plant-associated microbes and/or the plurality of collected plant-associated microbes is cultured for at two days.

In some cases, the method further comprises exposing the collected microbes to a mutagen prior to performing the final steps. In some cases, the mutagen is selected from the group consisting of: a chemical mutagen, ionizing radiation, and ultraviolet radiation. In some cases, the plurality of putative plant-associated microbes comprises wildtype strains. In some cases, the plurality of putative plant-associated microbes comprises microbes isolated from an environmental sample. In some cases, the plurality of putative plant-associated microbes comprises a library of strains formed by mutagenesis. In some cases, the plurality of putative plant-associated microbes comprises one or more strains with defects in DNA repair. In some cases, the plurality of putative plant-associated microbes comprises genetically modified microbes. In some cases, plurality of putative plant-associated microbes comprises one or more endophytes. In some cases, the plurality of putative plant-associated microbes comprises one or more epiphytes. In some cases, the plurality of putative plant-associated microbes comprises one or more rhizospheric microbes.

In some embodiments, the present disclosure provides a method of generating a variant microbial strain having altered plant colonization activity as compared to a parent microbial strain of the variant microbial strain, the method comprising: introducing genetic variations into a parent microbial strain, thereby producing a plurality of variant microbial strains; culturing the plurality of variant microbial strains in a plant exudate media (PEM); and isolating a variant microbial strain having altered growth in the PEM as compared to the parent microbial strain from the plurality of variant microbial strains. In some cases, the plurality of genetic variant microbial strains is cultured in the PEM as a community. In some cases, the altered plant colonization activity is improved plant colonization activity. In some cases, the altered plant colonization activity is worsened plant colonization activity. In some cases, the method further comprises repeating steps at least two, three, four, or five times. In some embodiments, the present disclosure provides a method of conducting a field trial of a plant beneficial microbial strain, comprising: culturing a plurality of plant beneficial microbial strains in a plant exudate medium (PEM); assaying an in vitro phenotype of the plurality of plant beneficial microbial strains; selecting a plant beneficial microbial strain that exhibits a desired in vitro phenotype; contacting the selected plant beneficial microbial strain with plants in a field; and assessing a plant phenotype of the plants in the field as compared to similar plants in a similar field which are not contacted with the selected plant beneficial microbial strain. In some cases, the plurality of plant beneficial microbial strains comprises a plurality of different species of microbes. In some cases, the plurality of plant beneficial microbial strains comprises a plurality of genetic variants of a single microbial species. In some cases, the method further comprises selecting a plant beneficial microbial strain when the desired in vitro phenotype is high titer growth in PEM. In some cases, the method further comprises selecting a plant beneficial microbial strain when the desired in vitro phenotype is a rapid growth rate in PEM.

In some embodiments, the present disclosure provides a method of conducting a field trial of a plant beneficial microbial strain, comprising: culturing a plant beneficial microbial strain in a plant exudate medium (PEM); assaying an in vitro phenotype of the plant beneficial microbial strain; if the in vitro phenotype is within a given range, contacting the plant beneficial microbial strain with plants in a field; and assessing a plant phenotype of the plants in the field as compared to similar plants in a similar field which are not contacted with the plant beneficial microbial strain. In some cases, the in vitro phenotype is a growth rate. In some cases, the plant phenotype is a yield of the plant. In some cases, the plants are cereal plants. In some cases, the method further comprises identifying microbes naturally associated with the plants; assaying the microbes for growth in the PEM; and identifying a microbe with a desired growth rate in the PEM. In some cases, the method further comprises introducing a genetic variation into one or more microbes naturally associated with the plants. In some embodiments, the present disclosure provides a method of improving growth of a plant, the method comprising exposing the plant to a microbe that has a desired growth rate in PEM.

In some cases, the desired growth rate is a rate of growth of the microbe which has previously been associated with improved growth of the plant. In some cases, the desired growth rate is determined by: (i) identifying a microbe that is able to colonize the plant; (ii) assaying the microbe for growth in PEM; (iii) assaying the impact of the microbe on growth of the plant; (iv) determining the desired growth rate of the microbe in PEM as a growth rate of the microbe in PEM that is associated with improved growth of the plant. In some cases, the method further comprises between (iii) and (iv), introducing a genetic mutation into the microbe. In some cases, the method further comprises selecting the microbe that has a desired growth rate in PEM by: (i) identifying one or more microbes that are able to colonize the plant; (ii) assaying the one or more microbes for growth in PEM; (iii) assaying the impact of the one or more microbes on growth of the plant; and (iv) determining a microbe with a desired growth rate in PEM as a microbe of the one or more microbes that is associated with improved growth of the plant. In some cases, the method further comprises, introducing a genetic mutation into the microbe after step (iii) to create a genetically modified microbe, and repeating steps (i) to (iii) with the genetically modified microbe. In some cases, the growth of the plant is measured by a yield of the plant. In some cases, the yield of the plant is measured by a yield of a grain produced by the plant. In some cases, the microbe is a diazotroph. In some cases, the microbe is a phosphate-solubilizing microbe. In some cases, the microbe is a genetically altered microbe. In some cases, the microbe is an epiphyte. In some cases, the microbe is an endophyte. In some cases, the microbe is rhizospheric. In some cases, the plant is a cereal plant.

In some cases, the plant is selected from the group consisting of: corn, soybean, canola, sorghum, potato, rice, barley, fonio, oats, Palmer's grass, rye, pearl millet, sorghum, spelt, teff, triticale, wheat, breadnut, buckwheat, cattail, chia, flax, grain amaranth, hanza, quinoa, and sesame. In some cases, the plant is selected from the group consisting of: corn, wheat, and rice.

In some embodiments, the present disclosure provides a method of predicting a phenotype of a plant grown with a microbial strain, the method comprising: culturing a microbial strain in a plant exudate medium (PEM); assaying an in vitro phenotype of the microbial strain; and using the in vitro phenotype from (b) to predict an phenotype of a plant grown with the microbial strain. In some cases, the in vitro phenotype of the microbial strain is microbial growth in PEM. In some cases, the phenotype of the plant grown with the microbial strain is plant growth. In some cases, the phenotype of the plant grown with the microbial strain is plant yield. In some cases, the plant grown with the microbial strain is a cereal plant. In some cases, the plant grown with the microbial strain is selected from the group consisting of: corn, soybean, canola, sorghum, potato, rice, barley, fonio, oats, Palmer's grass, rye, pearl millet, sorghum, spelt, teff, triticale, wheat, breadnut, buckwheat, cattail, chia, flax, grain amaranth, hanza, quinoa, and sesame.

In some cases, the PEM is a natural PEM (NPEM). In some cases, the NPEM is formed by steeping a root system of a plant in an aqueous solution. In some cases, the NPEM is formed by steeping a root system of a plant in an aeroponic system. In some cases, the NPEM is formed by steeping a root system of a plant in semisolid agar. In some cases, the NPEM is formed by steeping a root system of a plant on an absorbent surface. In some cases, the NPEM is formed by steeping a root system of a plant on an adsorbent surface. In some cases, the NPEM is formed by homogenizing a plant part in an aqueous solution. In some cases, the NPEM is formed by homogenizing a plant root system in an aqueous solution. In some cases, the plant is a cereal plant. In some cases, the plant is selected from the group consisting of: corn, soybean, canola, sorghum, potato, rice, barley, fonio, oats, Palmer's grass, rye, pearl millet, sorghum, spelt, teff, triticale, wheat, breadnut, buckwheat, cattail, chia, flax, grain amaranth, hanza, quinoa, and sesame. In some cases, the plant is selected from the group consisting of: corn, wheat, and rice. In some cases, the PEM is a synthetic PEM.

In some embodiments, the present disclosure provides an engineered microbe which comprises a modification which alters chemoattraction to a component of a plant exudate medium (PEM). In some cases, the altered chemoattraction is improved chemoattraction. In some cases, the altered chemoattraction is decreased chemoattraction. In some cases, the engineered microbe is a diazotrophic bacterium. In some cases, the engineered microbe is a phosphate-solubilizing bacterium. In some cases, the PEM is a natural PEM (NPEM). In some cases, the NPEM is formed by steeping a root system of a plant in an aqueous solution. In some cases, the NPEM is formed by steeping a root system of a plant in an aeroponic system. In some cases, the NPEM is formed by steeping a root system of a plant in semisolid agar. In some cases, the NPEM is formed by steeping a root system of a plant on an absorbent surface. In some cases, the NPEM is formed by steeping a root system of a plant on an adsorbent surface. In some cases, the NPEM is formed by homogenizing a plant part in an aqueous solution. In some cases, the NPEM is formed by homogenizing a plant root system in an aqueous solution. In some cases, the plant is a cereal plant. In some cases, the plant is selected from the group consisting of: corn, soybean, canola, sorghum, potato, rice, barley, fonio, oats, Palmer's grass, rye, pearl millet, sorghum, spelt, teff, triticale, wheat, breadnut, buckwheat, cattail, chia, flax, grain amaranth, hanza, quinoa, and sesame. In some cases, the plant is selected from the group consisting of: corn, wheat, and rice. In some cases, the PEM is a synthetic PEM.

In some embodiments, the present disclosure provides a method of selecting a microbial strain with an improved phenotype in planta, the method comprising: introducing genetic variations into a parent strain, thereby producing a library of genetic variant strains, culturing the genetic variant strains, and the parent strain, in plant exudate media, assaying a phenotype of the genetic variant strains in plant exudate media, selecting a genetic variant strain which shows an improvement in the phenotype in plant exudate media compared to the parent strain under similar conditions; thereby selecting a microbial strain with an improved phenotype in planta. In some cases, the phenotype is nitrogen fixation activity. In some cases, the assay for nitrogen fixation is an acetylene reduction assay. In some cases, the phenotype is ammonium excretion. In some cases, the assay for ammonium excretion is an ammonium excretion assay. In some cases, the phenotype is plant colonization ability. In some cases, the assay is a plant colonization assay. In some cases, the phenotype is rhizosphere fitness. In some cases, the assay is a cell growth competition assay. In some cases, the cell growth competition assay is conducted in vitro. In some cases, the cell growth competition assay is conducted a 96 well plate. In some cases, the cell growth competition assay is conducted in a flask. In some cases, the cell growth competition assay is conducted in planta in a hydroponic system. In some cases, the cell growth competition assay is conducted in planta. In some cases, the cell growth competition assay is conducted in planta in a grow room or greenhouse. In some cases, the cell growth competition assay is conducted in planta in a field. In some cases, the two or more cell growth competition assays are conducted under different environmental conditions. In some cases, the cell growth competition assay is conducted a 96 well plate. In some cases, the genetic variations are produced by random mutagenesis. In some cases, the genetic variations are produced using a transposon. In some cases, the genetic variations are produced by site directed mutagenesis. In some cases, the microbe is an endophyte. In some cases, the microbe is an epiphyte. In some cases, the microbe is a rhizophyte. In some cases, the plant exudate media is a natural plant exudate media. In some cases, the natural plant exudate media is formed by culturing a root system of a plant in an aqueous solution. In some cases, the natural plant exudate media is formed by culturing a root system of a plant in an aeroponic system. In some cases, the natural plant exudate media is formed by culturing a root system of a plant in semisolid agar. In some cases, the natural plant exudate media is formed by culturing a root system of a plant on an absorbent surface. In some cases, the natural plant exudate media is formed by culturing a root system of a plant on an adsorbent surface. In some cases, the plant is a cereal plant. In some cases, the plant is selected from the group consisting of: corn, soybean, canola, sorghum, potato, rice, barley, fonio, oats, palmer's grass, rye, pearl millet, sorghum, spelt, teff, triticale, wheat, breadnut, buckwheat, cattail, chia, flax, grain amaranth, hanza, quinoa, and sesame. In some cases, the plant is selected from the group consisting of: corn, wheat and rice.

In some embodiments, the present disclosure provides a method of evolving a microbial strain with improved plant colonization activity as compared to a parent strain of the evolved microbial strain, the method comprising: (a) introducing genetic variations into a parent strain, thereby producing a library of genetic variant strains, (b) culturing the genetic variant strains in plant exudate media as a community, and (c) isolating an evolved microbe from the genetic variant strains, wherein the isolated evolved microbe has improved plant colonization activity as compared to the parent strain. In some cases, the method further comprises repeating steps (b) and (c) at least two times. In some cases, the method further comprises repeating steps (b) and (c) at least three times. In some cases, the method further comprises repeating steps (b) and (c) at least four times. In some cases, the method further comprises repeating steps (b) and (c) at least five times. In some embodiments, the present disclosure provides an engineered microbe which has been modified to have increased chemoattraction to a component of plant exudate media. In some cases, the microbe is a diazotrophic bacterium. In some cases, the microbe is a phosphate solubilizing bacterium.

In some embodiments, the present disclosure provides a method of selecting a microbe which is attracted to a component of plant exudate media, the method comprising: obtaining a semisolid agar plate with multiple regions, the multiple regions comprising at least a first region comprising agar dissolved in a rich media; a last region comprising agar dissolved in plant exudate media; and a plurality of intermediate regions that each comprise a mix of rich media and plant exudate media thereby forming a gradient from rich media to plant exudate media; applying a plurality of microbes to the first region of the semisolid agar plate, culturing the semisolid agar plate for a period of time, and collecting microbes from the last region of the semisolid agar plate which has microbes. In some cases, the method further comprises repeating the steps to enrich for microbes attracted towards plant exudate media. In some cases, the semisolid agar plate is cultured for at least 16 hours. In some cases, the semisolid agar plate is cultured for at least two days. In some cases, the semisolid agar plate is cultured for at least one day. In some cases, the semisolid agar plate is cultured for at least 6 hours. In some cases, the method further comprises exposing the collected microbes to a mutagen prior to a repeat of steps (a)-(d). In some cases, the mutagen is selected from the group consisting of: chemical mutagens, ionizing radiation, and ultraviolet radiation. In some cases, the plurality of microbes comprises wildtype strains. In some cases, the plurality of microbes comprises a mutagenesis library of strains. In some cases, the plurality of microbes comprises one or more strains with defects in DNA repair. In some cases, the plurality of microbes comprises microbes isolated from an environmental sample. In some cases, the plurality of microbes comprises a library of microbial strains. In some cases, the plurality of microbes comprises a library of genetic variants.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates growth of K. variicola on synthetic and natural exudate media. All media was supplemented with 10 mM glutamine.

FIG. 1B illustrates growth of K. sacchari on synthetic and natural exudate media. All media was supplemented with 10 mM glutamine.

FIG. 1C illustrates growth of R. aquatilis on synthetic exudate media. All media was supplemented with 10 mM glutamine.

FIG. 2 illustrates results of an acetylene reduction assay (ARA) with K. variicola strain 137-2084 in control media (ARA) and in synthetic exudate media FN1.0. All media was supplemented with 5 mM ammonium phosphate.

FIG. 3 illustrates results of an amino acid analysis of water exposed to a corn root sample.

FIG. 4 illustrates results of an amino acid analysis of water exposed to a corn root sample.

FIG. 5A shows peak optical density values for three strains grown in greenhouse slurry media.

FIG. 5B shows colonization levels of the three microbes from FIG. 5A when grown on maize roots.

FIG. 6A shows peak optical density values for five strains grown in greenhouse slurry media.

FIG. 6B shows colonization levels of the five microbes from FIG. 6A when grown on maize roots.

FIG. 7A shows peak optical density values for two strains grown in greenhouse slurry media.

FIG. 7B shows colonization levels of the two microbes from FIG. 7A when grown on maize roots.

FIG. 8 shows doubling times for the five strains of FIG. 6A grown in greenhouse slurry media.

FIG. 9A shows titers of three strains grown in greenhouse slurry media in a HTP plate reader.

FIG. 9B shows titers of three strains grown in greenhouse slurry media in a micro-fermenter format.

FIG. 9C shows colonization levels of the three microbes from FIGS. 9A and 9B when grown on maize roots.

FIG. 10A shows doubling times for three strains grown in greenhouse slurry media.

FIG. 10B shows colonization levels of the three microbes from FIG. 10A when grown on maize roots.

FIG. 11A shows doubling times for two strains grown in grow room slurry media.

FIG. 11B shows fresh weight of plants inoculated with each of the strains in FIG. 11A.

FIG. 12A shows titers of three microbial species grown in grow room slurry media.

FIG. 12B shows doubling times of the three microbial species of FIG. 12A when grown in grow room slurry media.

FIG. 12C shows colonization levels of the three microbial species from FIG. 12A when grown on maize roots in a field. Cells/g FW refers to cells per gram of root fresh weight.

FIG. 12D shows results of a second field trial with the microbial species of FIG. 12C.

### DETAILED DESCRIPTION

As used herein, “in planta” generally refers to in, on, or in the vicinity of a plant. For example, a bacterium grown in planta may colonize an interstitial space of a plant, a cell of a plant, the surface of a plant or may grow in the rhizosphere of a plant. The plant may comprise leaves, roots, stems, seed, ovules, pollen, flowers, fruit, etc. The term in planta may also be used to describe a microbe grown in or on media which also contains a plant or plant part such as the roots of a plant.

The term “polynucleotide,” as used herein, generally refers to a molecule comprising a plurality of nucleotides or nucleotide analogues. A polynucleotide may have a nucleotide (or nucleic acid) sequence. A polynucleotide can be a chain of nucleotides of any length, and can comprise deoxyribonucleotides, ribonucleotides, or analogs thereof. A polynucleotide may have any three-dimensional structure, and may perform any function, known or unknown. Non-limiting examples of polynucleotides can include: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, the term “expression” generally refers to the process by which a polynucleotide can be transcribed from a deoxyribonucleic acid (DNA) molecule (such as into and mRNA or other RNA transcript) and/or the process by which a transcribed mRNA can be subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “polypeptide” generally refers to polymers of amino acids. The polymer may be linear or branched, it may comprise modified amino acids, and/or it may be interrupted by non-amino acids. The term can also encompass an amino acid polymer that has been modified; for example, via disulfide bond formation, glycosylation, lipidation, acetylation, phosphorylation, or any other manipulation, such as conjugation with a labeling component.

As used herein the term “amino acid” generally refers to natural and/or unnatural or synthetic amino acids, including glycine and both the D and/or L optical isomers, amino acid analogs, and/or peptidomimetics.

As used herein, the term “about” is generally used synonymously with the term “approximately.” The use of the term “about” with regard to an amount generally refers to values slightly outside the cited values, e.g., plus or minus 0.1% to 10%.

As used herein, the terms “biologically pure culture” or “substantially pure culture” generally refer to a culture of a bacterial species described herein containing no other bacterial species in quantities sufficient to interfere with the replication of the culture or be detected by normal bacteriological techniques.

As used herein, the term “heterologous” in the context of a gene or gene fragment generally refers to a gene or gene fragment which is present in a non-native context. In some cases, a non-native context may be a non-native cell. In some cases, a non-native context may be within the native cell of the gene or gene fragment, but in a non-native genomic location. In some cases, a heterologous promoter may be a promoter which has been moved within a bacterial strain such that it now regulates a coding sequence which it does not natively regulate.

### Root Exudate Nutrients

The root exudates of a plant (e.g., such as a cereal crop) may form a nutrient source for associated rhizospheric and root-associated bacteria, fungi, or other microbes or microorganisms. These exudates generally contain a mixture of simple and complex polysaccharides, organic acids, mucilages, phenolic compounds, fatty acids, sterols, vitamins, and amino acids. The nutrients provided by the root exudates can serve multiple functions including the attraction and/or maintenance of microbes that in turn may: directly provide or solubilize key nutrients to the plant (such as, but not limited to, nitrogen or phosphate); synthesize phytohormones such as auxins and cytokinins; regulate plant hormone levels; and/or provide localized pathogen control and other biocontrol of fungal and bacterial diseases. In some cases, components of such root exudates may include, but are not limited to, the components listed in Table 1.

TABLE 1
Some components of plant exudates

| Sugars | Organic Acids | Amino Acids | Fatty Acids | Sugar Acids | Sugar Alcohols | Vitamins |
|---|---|---|---|---|---|---|
| Glucose | Lactic | Glu | Adipic acid | Threonic acid | Myo-inositol | Thiamine |
| Arabinose | Oxalic | Asp | Palmitic acid | d-Arabinonic acid | Xylitol | Riboflavin |
| Fructose | Fumaric | Ala | Stearic acid | Gluconic acid | | Pyridoxine |
| Sucrose | Malic | gamma-amino | Palmitoleic acid | Gluconic acid | | Niacin |
| Melibiose | Citric | butyric Acid | Adipic acid | | | Pantothenic Acid |
| Maltose | Succinic | Ser | Oleic acid | | | |
| Lactose | Benzoic | Arg | Stearic acid | | | |
| Isomaltose | Aconitic | Gln | Linoleic acid | | | |
| Mannose | t-aconitic | Val | | | | |
| Glycerol | Tartaric | Leu | | | | |
| Trehalose | Glutamic | His + Gly | | | | |
| Inositol | Fumaric | Tyr | | | | |
| Psicose | Malonic | Phe | | | | |
| Sorbose | Aspartic | Glutamate | | | | |
| Rhamnose | butanoic | Thr | | | | |
| | acetoacetic | Isoleucine | | | | |
| | | Lys | | | | |
| | | Aspartate | | | | |

Generally, a root exudate can be a mixture of simple and complex polysaccharides, organic acids, mucilages, phenolic compounds, fatty acids, sterols, vitamins, and/or amino acids. These exudates can comprise one or more sugars, one or more organic acids, one or more amino acids, one or more fatty acids, one or more sugar acids, one or more sugar alcohols, one or more vitamins, or a combination thereof.

In some cases, a root exudate can comprise sugars. A sugar can be, for example, glucose, arabinose, fructose, sucrose, melibiose, maltose, lactose, isomaltose, mannose, glycerol, trehalose, inositol, psicose, sorbose, rhamnose, or any other suitable sugar.

In certain cases, a root exudate can comprise an organic acid. An organic acid can be, for example, lactic acid, oxalic acid, fumaric acid, malic acid, citric acid, succinic acid, benzoic acid, aconitic acid, t-aconitic acid, tartaric acid, glutamic acid, fumaric acid, malonic acid, aspartic acid, butanoic acid, acetoacetic acid, or any other suitable organic acid.

In various cases, a root exudate can comprise an amino acid. An amino acid can be, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, or any other suitable amino acid.

In some cases, a root exudate can comprise a fatty acid. A fatty acid can be, for example, adipic acid, palmitic acid, stearic acid, palmitoleic acid, adipic acid, oleic acid, stearic acid, linoleic acid, or any other suitable fatty acid.

In certain cases, a root exudate can comprise a sugar acid. A sugar acid can be, for example, threonic acid, d-arabinoic acid, gluconic acid, or any other suitable sugar acid.

In various cases, a root exudate can comprise a sugar alcohol. A sugar alcohol can be, for example, myo-inositol, xylitol, or any other suitable sugar alcohol.

In some cases, a root exudate can comprise a vitamin. A vitamin can be, for example, thiamine, riboflavin, pyridoxine, niacin, pantothenic acid, or any other suitable vitamin.

Microbes may be cultured and/or assayed in media which includes plant exudates or in media which includes components that mimic, or that are designed to mimic, plant exudates. Such a medium may be referred to as a plant exudate medium (PEM). PEM may form a proxy in which the microbes may be cultured in vitro, but with conditions that may better mimic those that the microbes may encounter in a field as compared to culturing the microbes in a conventional laboratory media.

In some embodiments, plant exudates may be collected by contacting, dipping, boiling, squeezing, steeping, homogenizing, grinding, culturing, or soaking one or more plants or plant parts (such as seedlings or plant root tissue) in an aqueous solution. Such an aqueous solution can be a water-based solution that can comprise buffer, salt, or other molecules in trace or larger amounts. In some cases, an aqueous solution can comprise only water. Water in such an aqueous solution can be tap water, distilled water, de-ionized water, spring water, filtered, water, or another type of water. In some cases, an aqueous solution may comprise one or more common agricultural fertilizers. In some cases, an aqueous solution may comprise one or more common agricultural herbicides or pesticides. Plant tissue may excrete and/or secrete carbon sources, amino acids, and/or other metabolites into its surroundings (e.g., into the aqueous solution). By collecting the aqueous solution contacted by such plant tissue (e.g., PEM), the microbes may be cultured in an environment or substrate which better mimics what the microbes may encounter in a field. PEM can be used as a culture medium for growing microbes. In some cases, PEM may mimic the growth condition of a microbe in a plant root environment (e.g., without growing the actual plant). In certain cases, the PEM can be obtained by grinding, chopping, grating, squeezing, agitating, vortexing, stirring, or otherwise homogenizing plant tissue, such as root tissue, to release compounds and/or metabolites into the aqueous solution (e.g., all or substantially all compounds and metabolites).

Root exudates in plants can promote microbe attraction. In some cases, the root exudates from plants can provide a chemical or nutrient gradient in the soil which can attract microbes to the root of the plant. PEM can be formulated to have a concentration of an ingredient that is consistent with that found somewhere along a natural gradient from a root. For example, in some cases, a concentration of an ingredient can be the same as the concentration of a root exudate near a root or the same as the concentration of a root exudate far from the root. In certain cases, a concentration of an ingredient can be the same as the concentration of the ingredient in a root exudate within 1 cm, within 5 cm, within 10 cm, within 20 cm, within 25 cm, or within 30 cm of a root. In various cases, a concentration of an ingredient can be the same as the concentration of the ingredient in a root exudate at least 1 cm, at least 10 cm, at least 20 cm, at least 25 cm, or at least 30 cm from a root. In some cases, a concentration of an ingredient can be the same as the concentration of the ingredient in a root exudate about 1 cm, about 10 cm, about 20 cm, about 25 cm, or about 30 cm from a root.

In some cases, the concentration of an ingredient can be the same as the concentration of the ingredient in a root exudate from 1 cm to 30 cm from a root, from 1 cm to 25 cm from a root, from 1 cm to 20 cm from a root, from 1 cm to 15 cm from a root, from 1 cm to 10 cm from a root, from 1 cm to 5 cm from a root, from 5 cm to 30 cm from a root, from 5 cm to 25 cm from a root, from 5 cm to 20 cm from a root, from 5 cm to 15 cm from a root, from 5 cm to 10 cm from a root, from 10 cm to 30 cm from a root, from 10 cm to 25 cm from a root, from 10 cm to 20 cm from a root, from 10 cm to 15 cm from a root, from 15 cm to 30 cm from a root, from 15 cm to 25 cm from a root, from 15 cm to 20 cm from a root, from 20 cm to 30 cm from a root, from 20 cm to 25 cm from a root, or from 25 cm to 30 cm from a root.

In some cases, a root exudate or PEM can promote the growth and/or maintenance of microbes that can provide (e.g., directly provide) or solubilize nutrients to the plant. In certain cases, microbes can provide or solubilize nitrogen, phosphate, or other nutrients. In various cases, microbes can synthesize phytohormones, for example, auxins and cytokinins. In some cases, microbes can regulate plant hormone levels. In certain cases, microbes can provide localized pathogen control or biocontrol of fungal and/or bacterial diseases of the plant.

Microbes attracted to, or maintained by, root exudate of a plant (e.g., a crop such as a cereal crop) can subsist on the root exudate. Such microbes can utilize soil-based nutrients, which may be needed for growth or nutrient production (e.g., nitrogen fixation). In some cases, the soil-based nutrients such microbes can utilize may include, for example, phosphate potassium, sulphate, nitrogen, oxygen, hydrogen, magnesium, calcium, boron, and chlorine, or metals such as molybdenum, iron, vanadium, copper, manganese, zinc, and nickel.

In comparison to root exudates, conventional bacterial laboratory media generally include one or more simple sugars (e.g., glucose or sucrose) and a nitrogen source. In some cases, other nutrients such as trace metals, phosphates, and vitamins may be added.

Bacterial metabolism may be strongly influenced by the combined nutrient, temperature, pressure, humidity, and oxygen environment, as well as competitive influences including the presence of other secretions by plants and microbes. Changes in metabolism in turn may influence the fitness of the microbe in a wide range of environments.

In some cases, microbial fitness may be defined as increased survival rates (viability) on the root system (endophytic and/or epiphytic) and/or in the rhizosphere of a plant in various stages of plant growth. Microbial fitness may be defined as increased proliferation (colonization) on the root system (endophytic and/or epiphytic) and/or in the rhizosphere of a plant in various stages of plant growth. Microbial fitness may be defined as improved persistence of the target microbe(s) after a given stage of plant growth. For example, microbial fitness may be defined as improved persistence of the target microbe(s) after stage V5 of corn growth. Microbial fitness may be defined as improved inter-species competitiveness in natural and synthetic communities in the above environments. Furthermore, microbial fitness may be defined as changes in gene expression for markers relating to improved nutrient utilization, attachment and infiltration of roots (including biofilm production), growth rates and tolerance of temperature, oxygen, pH, osmolality, and/or desiccation conditions.

Since defined laboratory media can differ significantly from root exudates, the use of synthetic or natural root exudates may provide an improved model for screening microbes in vitro for fitness as defined above. In some cases, the use of synthetic or natural root exudates may provide an improved model for screening microbes for different phenotypes which are desired when the microbe is cultured in planta.

Screening phenotypes of microbes in the presence of natural or synthetic plant exudates may better predict the phenotypes of the microbes when grown in planta. In some cases, different PEM may be selected to better model the microbe's microenvironment in, on, or around, different types of plants, or different developmental stages of plants.

In some cases, a natural PEM (NPEM) may be generated by contacting an aqueous solution with a plant or a portion of a plant. For example, a natural PEM may be prepared by dipping, chopping, grating, squeezing, agitating, vortexing, stirring, culturing, or soaking seedlings or plant root tissue in aqueous solution. Plant roots may excrete carbon sources, amino acids, and/or other metabolites into the aqueous solution. Culturing microbes in the aqueous solution in which the seedlings or plant tissue were dipped or cultured (e.g., in the natural PEM) can allow the microbes to be grown in a substrate which may better mimic or represent the environment the microbes may encounter in the plant rhizosphere in a greenhouse or field. In some cases, plant exudates can be obtained by grinding or otherwise homogenizing root tissue in an aqueous solution to release carbon sources, amino acids, metabolites, etc. into the aqueous solution for microbial culture or growth.

In certain cases, a medium which comprises plant exudates such as a natural PEM may be generated by mixing, chopping, grinding, homogenizing, shaking, vortexing, submerging, or steeping plant tissue in an aqueous medium, or otherwise exposing an aqueous medium to plant tissue. Such exposure or mixing can yield a medium which is a liquid, slurry, gel, mixture, solution, or paste.

In various cases, a synthetic PEM (SPEM) may be a medium which mimics a natural PEM. A synthetic PEM may comprise one or more components selected from Table 1. A synthetic PEM may comprise sugars, organic acids, amino acids, salts, plant hormones, and/or secondary metabolites. In some cases, a synthetic PEM may also comprise components commonly found in soil, such as nitrogen-containing compounds, agricultural fertilizers, pesticides, herbicides, and/or fungicides. Examples of several recipes for synthetic PEM are provided in Tables 2-4. In some cases, a synthetic PEM may comprise any combination of compounds and concentrations from Tables 2-4. The synthetic PEM may further comprise Na₂HPO₄ and/or KH₂PO₄. For example, the synthetic PEM may further comprise 25 g/L of Na₂HPO₄ and/or 3 g/L KH₂PO₄. In some cases, PEM can be filter sterilized. PEM can be stored at room temperature or at any other suitable temperature. PEM can be stored at about 4° C. In certain cases, storage of PEM at about 4° C. can cause one or more components to fall out of solution.

TABLE 2
Example synthetic PEM
Synthetic Exudate BD1.0

| Ingredient | g/L |
|---|---|
| Glucose | 3.314944 |
| Fructose | 3.314944 |
| Sucrose | 3.14916 |
| Lactate | 1.657472 |
| Citric Acid | 1.767504 |
| Succinic | 1.629642 |
| Alanine | 0.81972 |
| Serine | 0.57805 |
| Glutamic Acid | 1.61843 |

TABLE 3
Example synthetic PEM
Synthetic Exudate KK1.0

| Ingredient | g/L |
|---|---|
| Glucose | 2.2110 |
| Fumaric Acid | 1.1430 |
| Oxalic Acid | 0.6120 |
| Arabinose | 0.8610 |
| Fructose | 0.7320 |
| Sucrose | 0.4770 |
| Succinic Acid | 0.0960 |
| Citric Acid | 0.1440 |
| Malic Acid | 0.0810 |
| Benzoic Acid | 0.0600 |
| Alanine | 0.0178 |
| Aconitic | 0.0300 |
| Glutamic Acid | 0.0212 |
| Asparagine | 0.0190 |
| Tartaric Acid | 0.0210 |
| gamma-amino butyric Acid (GABA) | 0.0090 |
| Aspartic Acid | 0.0117 |
| Serine | 0.0079 |
| Arginine | 0.0093 |
| Leucine | 0.0052 |
| Valine | 0.0031 |
| Glutamine | 0.0037 |

TABLE 4
Example synthetic PEM
Synthetic Exudate FN1.0

| Ingredient | g/L |
|---|---|
| Malic Acid | 6.83859 |
| Melibiose | 6.67485 |
| Lactic Acid | 4.7292 |
| Succinic Acid | 4.428375 |
| Glucose | 4.0536 |
| Malonic Acid | 3.90225 |
| t-aconitic Acid | 3.13398 |
| Histidine | 2.0952 |
| Glycine | 1.01385 |
| Asparagine | 1.5972 |
| Leucine | 1.5744 |
| Glutamic Acid | 1.54455 |
| Tyrosine | 1.359 |
| Phenylalanine | 1.239 |
| Alanine | 1.20285 |
| Glutamate | 1.10325 |
| Maltose | 1.0269 |
| Lactose | 0.82152 |
| Valine | 0.7026 |
| Threonine | 0.53595 |
| Isomaltose | 0.5136 |
| Serine | 0.47295 |
| Isoleucine | 0.3936 |
| Fumaric Acid | 0.104463 |
| Fructose | 0.091882 |
| Lysine | 0.06579 |
| Trehalose | 0.056496 |
| Mannose | 0.041617 |
| Glycerol | 0.016302 |
| Inositol | 0.012972 |
| Arabinose | 0.005855 |

The fitness of wild type or remodeled/engineered/mutated/evolved microbes may be evaluated by using either synthetic or natural root exudate in in vitro assays to mimic the rhizosphere and root environment of cereal crops at different growth stages in an agricultural field environment. In some cases, in vitro cultures may be grown in liquid or semisolid PEM in sealed glass vials, tubes, flasks, or cell culture plates (e.g., 96- or 384-well plates) under any of the conditions described herein. In some cases, the temperature, oxygen content, pressure, and/or pH of the in vitro environment may be modified. For example, the temperature may range from about 4° C. to about 45° C., about 10° C. to about 40° C., or about 15° C. to about 37° C., oxygen from 0.1% to 21% and pH from 4.5-8.5.

The oxygen content of such an in vitro environment can be at least 0.1% O₂, at least 0.5% O₂, at least 1% O₂, at least 5% O₂, at least 10% O₂, at least 15% O₂, at least 20% O₂, or at least 21% O₂. In some instances, the oxygen content can be no more than 0.1% O₂, no more than 0.5% O₂, no more than 1% O₂, no more than 5% O₂, no more than 10% O₂, no more than 15% O₂, no more than 20% O₂, or no more than 21% O₂. In various instances, the oxygen content can be about 0.1% O₂, about 0.5% O₂, about 1% O₂, about 5% O₂, about 10% O₂, about 15% O₂, about 20% O₂, or about 21% O₂.

The pH of such an in vitro environment can be about 4.5, about 5.0, about 5.5, about 6.0, about 6.5, about 7.0, about 7.5, about 8.0, or about 8.5. In some embodiments, the pH can be at least 4.5, at least 5.0, at least 5.5, at least 6.0, at least 6.5, at least 7.0, at least 7.5, at least 8.0, or at least 8.5. In various embodiments, the pH can be no more than 4.5, no more than 5.0, no more than 5.5, no more than 6.0, no more than 6.5, no more than 7.0, no more than 7.5, no more than 8.0, or no more than 8.5.

Natural plant exudate may be collected by exposing a carrier, for example, water, to a plant. In some cases, natural plant exudate may be collected by exposing a carrier, for example, water, to a maize plant. In certain cases, natural plant exudate may be collected at different plant growth stages through the following methods: hydroponic, transfer from soil, sand or other commonly used plant growth medium to a liquid capture system. In some cases, the natural exudate can be collected directly from the soil, sand, or other medium the plant is grown in (e.g., by filtering, vacuuming, suctioning, etc). In various cases, a natural exudate may be collected using an aeroponic system where root exudate is captured via a vacuum. A natural exudate may be captured in a semisolid medium, such as semisolid agar, or on a substrate such as a filter paper or another absorbent or adsorbent substrate. In some cases, natural exudate may be collected at different maize plant growth stages (from germination to V10) through the following methods: hydroponic, aeroponic, or transfer from soil, sand or other commonly used plant growth medium to a liquid capture system. In some cases, the natural exudate can be collected from the plant (e.g., by dipping, chopping, grating, squeezing, agitating, vortexing, stirring, culturing, or soaking dirt, sand, plant growth medium, seedlings, or plant root tissue in aqueous solution). In certain cases, the collected exudate may be concentrated through evaporation or other suitable methods.

In some embodiments, synthetic PEM may be prepared from recipes derived, for example, from chemical compositional analysis of collected natural exudate, literature, or other studies.

Both synthetic and natural PEM may be supplemented with nutrients commonly found in soil (e.g., as listed above), including, for example, commonly applied agricultural fertilizers, to more closely mimic different root and/or soil environments.

Microbial fitness may be measured in many different ways. In some cases, fitness may be measured by comparison of an in vitro proliferation, growth rate and/or viability of a monoculture to an on-root and/or in rhizosphere colonization and viability measurements. In some cases, fitness may be measured by comparison of the relative in vitro proliferation, growth rate and/or viability to on-root and/or rhizosphere colonization of the target microbe in the context of synthetic or natural microbial communities (for example, a microbial community that includes the target microbe(s) plus one or more other common soil and root bacteria and/or fungi). In some cases, root colonization may be induced by application of the microbe as a seed coating or in-furrow application at planting or after germination. In some cases, root colonization may be induced up to maize stage V5. In some cases, plants may be grown in a laboratory, greenhouse, or field environment. In some cases, viability and in vitro proliferation may be measured by dilution and plating on permissive media, staining with indicator dyes, flow cytometry, ATP based assays, protein quantification, transcriptional analysis, or any other method known in the art. In some cases, root colonization may be measured by quantitative PCR using species specific primers, 16s profiling and/or use of unique barcodes found naturally or introduced into the genome. In some cases, transcriptional profiling (RNA-Seq or similar) and metabolite analysis may also be used to evaluate fitness using known and novel indicators of microbial function. In some cases, analysis of mono and mixed cultures may include the use of fluorescent, protein, metabolite or other natural or introduced markers.

Plants (e.g., maize plants) can be grown in a laboratory, a greenhouse, or a field environment. In some cases, an in vitro proliferation, or growth rate, of a microbe in PEM may be compared with the root or rhizosphere colonization of the microbe. Colonization can be measured by dilution and plating on permissive media, staining with indicator dyes, flow cytometry, ATP-based assays, protein quantification, transcriptional analysis, or a combination thereof. In vitro proliferation can be measured by dilution and plating on permissive media, staining with indicator dyes, flow cytometry, ATP-based assays, protein quantification, transcriptional analysis, or a combination thereof. Root colonization can also be measured by quantitative PCR using species specific primers, 16s profiling, unique barcodes found naturally or introduced into the genome, or a combination thereof.

Described herein are methods for predicting an in planta phenotype of a microbial strain. Such methods can comprise culturing a microbial strain in a plant exudate medium and assessing an in vitro phenotype of the microbe. In some cases, the in vitro phenotype may be used to predict an in planta phenotype. For example, a microbial strain which grows well in PEM may colonize a plant root system better than a microbial strain which does not grow well in PEM. In some cases, a different phenotype can be measured in vitro than is measured in planta. In some cases, the predicted in planta phenotype may be a desirable phenotype or an undesirable phenotype.

Such methods can also comprise assaying an in vitro phenotype of the microbial strain. In some cases, the in vitro phenotype can be improved or worsened, for example, compared with a same microbe grown in the rhizospheric zone of a plant or near the root of a plant. In some cases, the in vitro phenotype can be improved or worsened, for example, compared with a same microbe found in nature (e.g., in a field, greenhouse, growth chamber, or hydroponic system), or compared with a parental strain. An in vitro phenotype can comprise a phenotype that the microbe displays when cultured (e.g., in a laboratory setting, or when cultured in a plant exudate medium but not in the presence of a root). Examples of in vitro phenotypes can include peak optical density of the culture, color, smell, protein expression, gene expression, growth rate, antimicrobial resistance properties, nitrogen fixation capabilities, ammonium excretion, colonization ability, or rhizosphere fitness.

Peak optical density can be measured as the maximum optical density of the microbe in a culture. Optical density can be a measure of the concentration of a bacteria in a suspension. In some cases, peak optical density can indicate the maximum concentration of bacteria that can occur in a culture under given conditions. Optical density can be measured in a spectrophotometer, for example, at 600 nm or another appropriate wavelength. In some cases, optical density can be measured during a mid-log phase of growth of a microbe. A peak optical density of a culture can be higher or lower than that of a same microbe grown under different conditions (e.g., different culturing conditions, in a field, in a growth chamber, in a hydroponic system, in a greenhouse, in a rhizospheric zone, etc.).

Color of the culture can be determined by sight, or can be measured, for example, by using a spectrophotometer. In some cases, color can be determined by pixel analysis of a photograph of the culture. Color of a culture can be different than that of a same microbe grown under different conditions (e.g., different culturing conditions, in a field, in a growth chamber, in a hydroponic system, in a greenhouse, in a rhizospheric zone, etc.).

Smell of the culture can be measured qualitatively during any phase of the growth of the microbe. In some cases, smell can be determined by a laboratory technician by wafting the air above the culture toward the nose. Smell of a culture can be different than that of a same microbe grown under different conditions (e.g., different culturing conditions, in a field, in a growth chamber, in a hydroponic system, in a greenhouse, in a rhizospheric zone, etc.).

Protein expression can be a measure of the amount of a single protein expressed in a culture, the total amount of protein expressed in a culture, the ratio of two or more proteins expressed in a culture, or a combination thereof. Protein expression can be measured as an absolute value (e.g., total amount of protein) or a relative value (e.g., normalized, compared with a protein expression of a same microbe grown under different or natural conditions, or compared with the expression of another protein in the culture). In some cases, expression of a protein useful for or essential for growth, antimicrobial resistance, nitrogen fixation capabilities, ammonium excretion, colonization ability, or rhizosphere fitness can be measured. In some cases, expression of a protein detrimental to growth, antimicrobial resistance, nitrogen fixation capabilities, ammonium excretion, colonization ability, or rhizosphere fitness can be measured. Protein expression can be measured on a protein sample collected from a culture using any suitable method. Protein expression can be measured via dot blot, western blot, a bicinchoninic acid assay, a Bradford assay, a fluorescent assay, fast protein liquid chromatography, or other suitable methods. Protein expression can be increased, decreased, or the same as that of a same microbe grown under different conditions (e.g., different culturing conditions, in a field, in a growth chamber, in a hydroponic system, in a greenhouse, in a rhizospheric zone, etc.).

Gene expression can be a measure of the amount of one or more genes expressed in a culture, the total amount of genes expressed in a culture, the ratio of two or more genes expressed in a culture, or a combination thereof. Gene expression can be measured as an absolute value (e.g., total amount of gene) or a relative value (e.g., normalized, compared with a gene expression of a same microbe grown under different or natural conditions, or compared with the expression of another gene in the culture). In some cases, gene expression can be measured as the amount of mRNA of a gene, the amount of mRNA of a plurality of genes, or total mRNA. In some cases, expression of a gene where the gene product is useful for or essential for growth, antimicrobial resistance, nitrogen fixation capabilities, ammonium excretion, colonization ability, or rhizosphere fitness can be measured. In some cases, expression of a gene where the gene product is detrimental to growth, antimicrobial resistance, nitrogen fixation capabilities, ammonium excretion, colonization ability, or rhizosphere fitness can be measured. Gene expression can be measured on a sample collected from a culture using any suitable method. In some cases, gene expression can be measured on extracted or purified mRNA from a culture. Gene expression can be measured via PCR, q-PCR, RT-PCR, in situ hybridization, or any other suitable method. Gene expression can be increased, decreased, or the same as that of a same microbe grown under different conditions (e.g., different culturing conditions, in a field, in a growth chamber, in a hydroponic system, in a greenhouse, in a rhizospheric zone, etc.).

Growth rate can be a measure of how fast or slow a culture is growing (i.e., how fast the microbe is replicating, or how fast the microbe is replicating combined with death rate of the microbe). Growth rate can be measured using optical density measurement techniques. In some cases, optical density can be measured at different time points while the microbe is being cultured, and the growth rate can be calculated. For example, optical density can be measured in increments of 5 minutes, 15 minutes, 30 minutes, 1 hour, 2 hours, 3 hours, 4 hours, 5 hours, 6 hours, 12 hours, 1 day, or a combination thereof. Growth rate can be increased, decreased, or the same as that of a same microbe grown under different conditions (e.g., different culturing conditions, in a field, in a growth chamber, in a hydroponic system, in a greenhouse, in a rhizospheric zone, etc.).

Antimicrobial resistance properties can be a measure of how well the culture can survive or grow in the presence of an antimicrobial agent. In some cases, antimicrobial resistance can be measured by measuring the growth rate of a culture in the presence of an antimicrobial agent. In some cases, antimicrobial resistance can be increased, decreased, or the same as that of a same microbe grown under different conditions (e.g., different culturing conditions, in a field, in a growth chamber, in a hydroponic system, in a greenhouse, in a rhizospheric zone, etc.).

Nitrogen fixation capabilities can be a measure of how well a microbe, or a microbial culture, can fix nitrogen. Nitrogen fixation capabilities can be measured as expression of proteins which can support nitrogen fixation, expression of genes which can support nitrogen fixation, or measurement of nitrogen fixation. Nitrogen fixation can be measured, for example, by an in planta assay or an acetylene reduction assay (ARA). Nitrogen fixation can be measured on a root, in a liquid dish, on an agar plate, in a cell culture plate (e.g., a 96-well plate), in a laboratory, in a greenhouse, in a field, in a growth chamber, in a hydroponic system, or in another appropriate location or vessel. Nitrogen fixation can be increased, decreased, or the same as that of a same microbe grown under different conditions (e.g., different culturing conditions, in a field, in a growth chamber, in a hydroponic system, in a greenhouse, in a rhizospheric zone, etc.).

Ammonium excretion can be an amount of ammonium (e.g., fixed nitrogen) which can exit a microbe or a culture into the microbe's environment. Ammonium excretion can be measured as expression of proteins which can support ammonium excretion, expression of genes which can support ammonium excretion, or measurement of ammonium excretion. Ammonium excretion can be measured ,for example, by an ammonium excretion assay or an in planta assay. Ammonium excretion can be measured on a root, in a liquid dish, on an agar plate, in a cell culture plate (e.g., a 96-well plate), in a laboratory, in a greenhouse, in a field, in a growth chamber, in a hydroponic system, or in another suitable location or vessel. Ammonium excretion can be increased, decreased, or the same as that of a same microbe grown under different conditions (e.g., different culturing conditions, in a field, in a growth chamber, in a hydroponic system, in a greenhouse, in a rhizospheric zone, etc.).

Colonization ability can be a measure of the ability of a microbe or culture to colonize an environment or to form a colony when applied to a root, field, liquid, soil, or plant. In some cases, a colonization ability can be a measure of how quickly a microbe can colonize, how dense or large a colony can be, or how fast-growing a colony can be. Colonization ability can be measured by a colonization assay. Colonization ability can be measured on a plant or plant part, e.g., a root, grown in a greenhouse, a field, a growth chamber, a hydroponic system, or in another suitable location or vessel.

Rhizospheric fitness can refer to the fitness of a rhizosphere, for example, of a plant. The rhizosphere can be a region of soil or media that can be influenced by root exudate and/or microbes present, either naturally or introduced, at or near the root. Rhizospheric fitness can refer to colonization of a microbe, diversity of microbes, quality of microbes, nitrogen fixed, ammonium excreted, nutrients present, oxygen available, water available, or another property of a rhizospheric zone. In some cases, rhizosphere fitness can be measured using a colonization assay (i.e., rhizosphere fitness can be correlated with colonization, for example, such that better rhizosphere fitness can correlate with better colonization). A colonization assay can be performed ,for example, in planta. Such an assay can be conducted in any suitable environment, such as a growth chamber, a hydroponic system, a greenhouse, a field, or another environment. In some cases, rhizosphere fitness can be assayed using a cell growth competition assay. A cell growth competition assay can measure replication fitness of a microbe. For example, a plurality of microbial strains can be seeded on a plant or in a medium and allowed to grow. The microbial strains can compete for cellular targets, nutrients, oxygen, or other commodities under identical conditions. In some cases, two or more cell growth competition assays can be conducted. Such two or more cell growth competition assays can be conducted under different conditions, such as different atmospheric conditions. For example, such two or more cell growth competition assays can be conducted under different temperature, humidity, precipitation, freeze/thaw, wind, nutrient availability, or other conditions. In some cases, three, four, five, six, or more cell growth competition assays can be conducted. Rhizospheric fitness can be increased, decreased, or the same as compared to the other microbial strains in the cell growth competition assay.

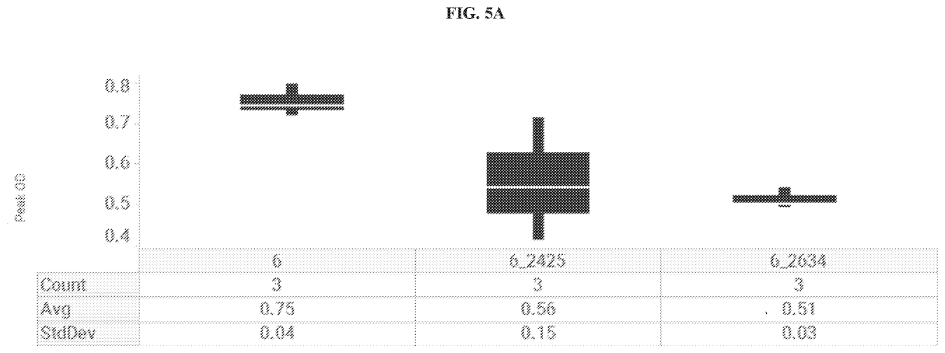

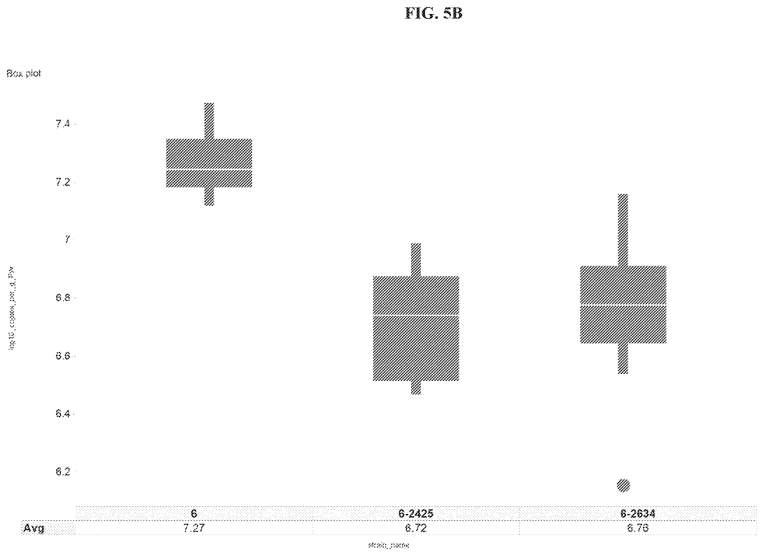

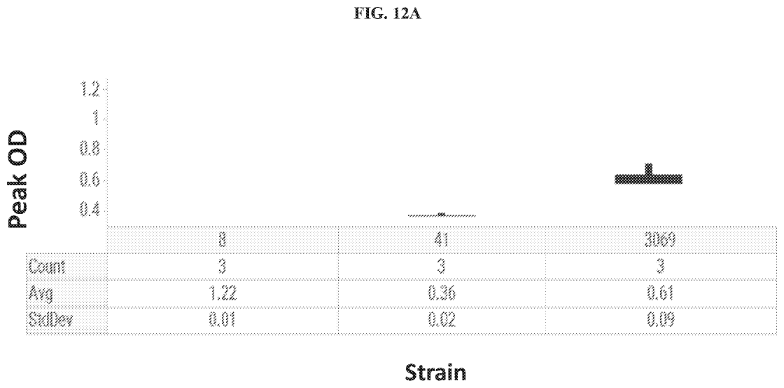

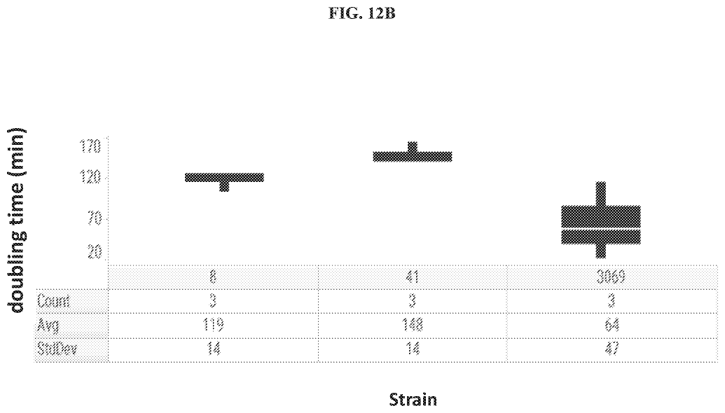

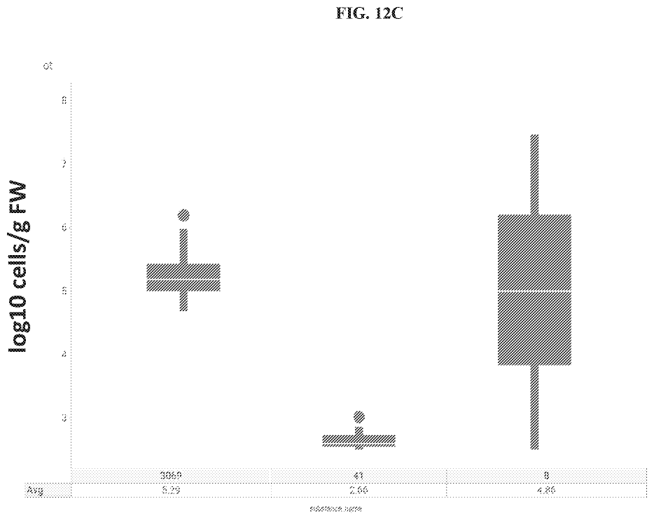

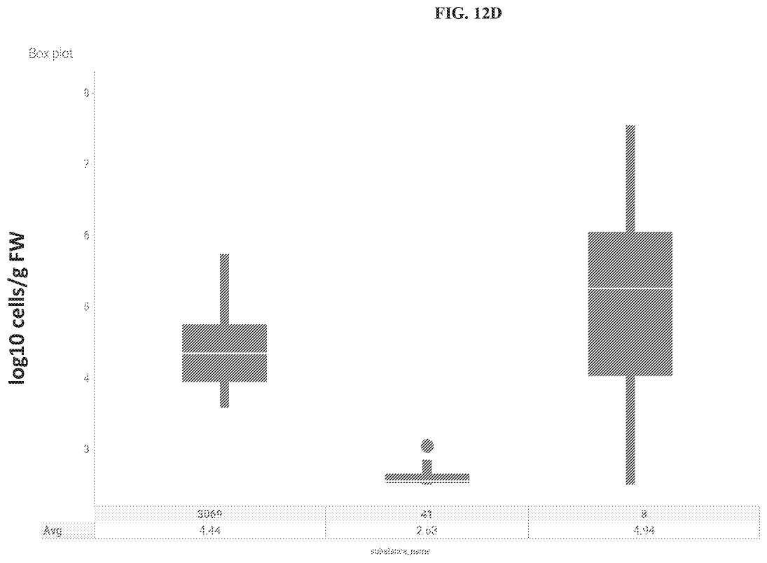

Such methods as provided herein can also comprise using an assayed in vitro phenotype to predict an in planta phenotype of the microbial strain. In some cases, the in vitro phenotype of a microbial strain can be indicative of the same phenotype in planta of the microbial strain. In some cases, the in planta phenotype can be directly correlated with the in vitro phenotype of the microbial strain. For example, FIG. 5A shows that two genetically modified strains, 6_2425 and 6_2634, grow less well in a PEM media than the parental strain, 6. FIG. 5B shows that both genetically modified strains of FIG. 5A are less able to colonize maize roots in a grow room than the parent strain. Similarly, in FIG. 12A three different bacterial species are assessed for titer and doubling time in a PEM. Strain P. polymixa 41 showed the lowest peak OD and the slowest doubling time of the three strains. Similarly strain P. polymixa 41 showed the lowest level of colonization of roots in greenhouse and field trials. Thus, indicating that in vitro phenotypes may be used to assess a plurality of microbial strains and may be used to select the strains which may have or exhibit desired in planta phenotypes.

In some cases, the in vitro phenotype of a microbial strain can be indicative of a different phenotype in planta of the microbial strain. In some cases, an increase in the measurement of a phenotype of a microbe in vitro can be indicative of an increase in the measurement of a different phenotype in planta. For example, an increase in the expression of a gene or expression of a protein that can support nitrogen fixation in vitro can be indicative of an increase of nitrogen fixation in planta. In some cases, an increase in the measurement of a phenotype of a microbe in vitro can be indicative of a decrease in the measurement of a different phenotype in planta. For example, an increase in the expression of a gene or expression of a protein that can hinder or inhibit nitrogen fixation in vitro can be indicative of a decrease in nitrogen fixation in planta. In some cases, a decrease in the measurement of a phenotype of a microbe in vitro can be indicative of a decrease in the measurement of a different phenotype in planta. For example, a decrease in the expression of a gene or expression of a protein that can support nitrogen fixation in vitro can be indicative of a decrease of nitrogen fixation in planta. In some cases, a decrease in the measurement of a phenotype of a microbe in vitro can be indicative of an increase in the measurement of a different phenotype in planta. For example, a decrease in the expression of a gene or expression of a protein that can hinder or inhibit nitrogen fixation in vitro can be indicative of an increase in nitrogen fixation in planta.

Also described herein are methods for selecting a genetically modified microbial strain having an altered in planta phenotype. The selection of the genetically modified microbial strain may be based on one or more in vitro phenotypes of the strain. In some cases, the in vitro phenotype tested may be the same as a desired in planta phenotype. For example, the in vitro phenotype may be ammonium excretion and the desired in planta phenotype may be high ammonium excretion. In some cases, the in vitro phenotype may be related to the in planta phenotype. For example, the in vitro phenotype may be growth rate or maximal growth and the in planta phenotype may be plant colonization. In some cases, a plurality of genetically modified microbial strains may be produced and screened for a preferred in vitro phenotype. In some cases, the genetically modified microbial strains may have genetic modifications which are expected to alter the in vitro phenotype. For example, a genetically modified microbial strain with a genetic alternation in a nitrogen fixation or nitrogen assimilation genetic regulatory network may be screened for in vitro nitrogen fixation or nitrogen assimilation activity. In some cases, the genetically modified microbial strains may have genetic modifications which are not expected to alter the in vitro phenotype. For example, a genetically modified microbial strain with a genetic alternation in a nitrogen fixation or nitrogen assimilation genetic regulatory network may be screened for in vitro growth rate or total growth in vitro to predict the in planta colonization of the strain.

Selection can comprise positive selection or negative selection. Positive selection can comprise selection of a microbial strain having an in planta phenotype that can be beneficial or desired. Such examples can include, but are not limited to, microbial strains that have improved or superior nitrogen fixation capabilities, ammonium excretion, colonization ability, rhizosphere fitness, or a combination thereof compared with an unmodified microbial strain and/or other modified microbial strains. Negative selection can comprise the non-selection of a microbial strain having an in planta phenotype that may not be beneficial or may not be desired. Such examples can include, but are not limited to, microbial strains that have worsened nitrogen fixation capabilities, ammonium excretion, colonization ability, rhizosphere fitness, or a combination thereof compared with an unmodified microbial strain and/or other modified microbial strains.

Further described herein are methods of selecting plant associated microbes which are attracted to PEM. Microbes attracted to PEM can migrate or move toward PEM when in the vicinity of PEM. In some cases, microbes which are attracted to PEM can grow better or faster in the presence of PEM. In some cases, microbes which are attracted to PEM can have better rhizospheric fitness compared with a microbe not attracted to PEM. In some cases, a microbe which is attracted to PEM and which has a plant beneficial trait may impart a greater benefit to a plant than a microbe with a similar plant beneficial trait but which is not attracted to PEM. For example, if a first nitrogen fixing microbe is attracted to PEM and a second nitrogen fixing microbe is not, the first and second microbe may both show the same level of nitrogen fixation activity when assayed in vitro (in either conventional media or PEM), but the first microbe may impart a greater growth advantage to a plant.

Also described herein are methods of conducting field trials of plant beneficial microbial strains. In some cases, a first step prior to beginning a field trial of a putative plant beneficial microbe may comprise assaying relevant phenotypes of the putative plant beneficial microbe in PEM. Relevant phenotypes may include, but are not limited to, growth rate, maximal OD, and titer in PEM. In some cases, relevant phenotypes may also include nitrogen fixation or ammonium excretion. In some cases, if a putative plant beneficial microbe shows very slow growth rate, low maximal OD, and low titer in PEM that microbe may not be selected for field trials. In some cases, a plurality of putative plant beneficial microbes may be screened for relevant phenotypes in PEM and those results may be used to select one or more microbes for field trials. In general, preferred in vitro phenotypes include, but are not limited to, rapid growth rate, high maximal OD, and high titer in PEM.

Also described herein are methods of improving growth (e.g., growth rate) of a plant. A method of improving a growth rate of a plant may comprise inoculating a plant, or soil in which a plant is to be grown, with a microbial strain that has a desired phenotype in PEM. In some cases, a method of improving growth of a plant may comprise exposing the plant to a microbe that has a desired growth rate in PEM. The desired growth rate may be a growth rate of a microbe which has previously been associated with improved growth of the plant. The desired growth rate may be determined by identifying a microbe that is able to colonize the plant; assaying the microbe for growth in PEM; assaying the impact of the microbe on growth of the plant; and determining the desired growth rate of the microbe in PEM as a growth rate of the microbe in PEM that is associated with improved growth of the plant. In some cases, this method may further comprise introducing a genetic mutation into the microbe.

In some cases, the method further comprises selecting the microbe that has a desired growth rate in PEM by (i) identifying one or more microbes that are able to colonize the plant; (ii) assaying the one or more microbes for growth in PEM; (iii) assaying the impact of the one or more microbes on growth of the plant; and (iv) determining a microbe with a desired growth rate in PEM as a microbe of the one or more microbes that is associated with improved growth of the plant. In some cases, if a desired microbe is not identified in the previous step the method may be iterated by introducing a genetic mutation into the microbe after step (iii) to create a genetically modified microbe, and repeating steps (i) to (iii) with the genetically modified microbe. This method may be iterated multiple times as required.

For example, a plant may be inoculated with a microbial strain which has a fast growth rate, high maximal OD, and/or high titer when grown in PEM. In some cases, improving growth of a plant may comprise increasing the fresh weight of the plant. In some cases, improving growth of a plant may comprise increasing the yield of the plant. In some cases, improving growth of a plant may comprise increasing the yield of a leaf, seed, grain, nut, fruit, and/or tuber produced by the plant.

Use of Synthetic or Natural Exudate Media to Better Predict Phenotypes in Planta (e.g. N Fixation)

Since most commonly used bacterial laboratory growth media differ significantly from root exudates, the use of synthetic exudate media may provide an improved model for an in vitro screen to predict bacterial phenotypes such as improved nitrogen fixation and/or excretion in-planta. Synthetic root exudates are advantageous in that they are highly consistent and easily modifiable—reducing noise due to media variability.

As stated above, bacteria subsisting on root exudates may also utilize soil-based nutrients required for both growth and nutrient production (such as nitrogen fixation). These soil-based nutrients may include but are not limited to phosphate, potassium, sulphate, nitrogen, oxygen, hydrogen, magnesium, calcium, boron and chlorine, and metals such as molybdenum, iron, vanadium, copper, manganese, zinc and nickel.

Improved bacterial phenotypes may be evaluated by using either synthetic root exudate media to mimic the rhizosphere and root environment of cereal crops at different growth stages in an agricultural field environment. In vitro cultures may be grown in exudate media in sealed glass vials, tubes, flasks, or cell culture plates (e.g., 96- or 384-well plates), in liquid or semisolid media under any of the conditions described above.

Improved nitrogen fixation and excretion may be measured by any suitable method. For example, improved nitrogen fixation may be measured by an ARA, improved nitrogen excretion may be measured by an ammonium excretion assay, and both may be measured by an in planta assay. In some cases, relative viability and growth rates may be measured on different synthetic exudates (+/− supplementation). These may be measured as a function of optical density, viable cell counts, ATP or enzymatic assay, or any other method known in the art. In some cases, in vitro nitrogen excretion as ammonium may be quantified using the Ammonium Excretion Assay, ammonium probes or any other assay known in the art. Nitrogen excretion can be correlated to in planta excretion through the use of exudate measurements, ammonia probes, enzymatic assays, ¹⁵N uptake, ¹⁵N isotope dilution, plant N metabolism gene markers, plant biomass assays, biosensors or any other method known in the art. In some cases, other forms of nitrogen excretion may be measured as described above, including excretion of amino acids. In some cases, in vitro nitrogen fixation may be quantified using the reduction of acetylene as a proxy using the ARA. This can be correlated to in planta fixation through the use of in planta ARA measurements, ¹⁵N uptake, ¹⁵N isotope dilution, plant N metabolism gene markers, plant biomass assays, biosensors or any other method known in the art.

In some cases, the relative nitrogen fixation and excretion of the target microbe may be quantified when grown in a synthetic or natural microbial community (for example, a microbial community including the target microbe plus one or more other common soil and root bacteria and/or fungi). Such results may be compared to in planta activity as described above. In some cases, transcriptional profiling (RNA-Seq or similar) and metabolite analysis may also be used to evaluate nitrogenase pathway expression or other related pathways both in vitro and in planta. In some cases, analysis of pure and mixed cultures may include the use of fluorescent, protein, metabolite or other natural or introduced markers.

In some cases, an in vitro phenotype of a microbe grown in PEM may be used to predict a phenotype of a plant grown with the microbe. For example, a putative plant beneficial microbe may be grown in PEM and assayed for an in vitro phenotype such as, e.g., titer or growth rate. A high titer or rapid growth rate may predict that a plant grown with the putative plant beneficial microbe will grow faster or larger than a plant grown with a different microbe that does not have the same phenotypes in vitro. In certain cases, the in vitro phenotype of the microbe may be able to predict the size, growth rate, and/or yield of a plant grown with the microbe. In some cases, the prediction may be of a relative quality, i.e., a first microbe out of a plurality of microbes may have a higher titer in PEM, or faster growth rate in PEM, compared to a second microbe, leading to a prediction that the first microbe will cause greater plant growth or greater plant yield when a plant is grown with the first microbe compared to the second microbe.

### Use as Bait for Chemotaxis Assay

Natural plant exudate media (NPEM) may be media made from exudate collected from live plants at various stages of growth. Synthetic plant exudate media (SPEM) may be media made from a variety of ingredients that is meant to mimic NPEM. Both types of media may contain polysaccharides, organic acids, amino acids and poly-peptides.

Plants are known to release chemoattractants to recruit different microbes to populate their rhizosphere. These microbes can form mutually beneficial relationships with the plant they migrate towards. NPEM can contain these chemoattractants which can be used to isolate microbes that are naturally recruited by plants to colonize their rhizosphere.

To determine which microbes are attracted to plant exudate a semisolid agar plate can be made with a mixture of rich media (such as LB media)/minimal media/water and NPEM. The first stage (or region) of the plate can 100% LB media, the second stage 90% LB and 10% NPEM, third stage 80% LB and 20% NPEM, etc. A soil sample can then be plated on one side of the plate and over time, microbes that are attracted to the NPEM may be seen migrating through the semisolid agar towards the area of greater NPEM concentration. In various cases, a semisolid agar plate may be obtained having multiple regions comprising a first region of agar dissolved in a rich medium, a last region with agar dissolved in PEM, and a plurality of intermediate regions that each comprise a mix of the rich medium and PEM to form a gradient from the first region to the last region. A plurality of putative plant-associated microbes may be applied to the first region and then cultured for a period of time. Microbes which are attracted towards PEM may migrate across the plate towards the PEM, and can be identified by collecting the one or more microbes which have migrated the furthest from the first region toward the last region. In some cases, plant-associated microbes may be attracted towards the PEM.

Described herein are methods of generating a variant microbial strain having altered plant colonization activity. SPEM and NPEM can be used to conduct an evolution experiment to create strains that are better suited for growth on plant exudate leading to an increase in colonization. Wildtype strains, a mutagenesis library of strains, or strains with defective DNA repair genes (mut genes) can be grown iteratively on NPEM/SPEM and then re-isolated. In some cases, a mutagenesis library of strains may be generated using a chemical mutagen, ionizing radiation, or ultraviolet radiation. Examples of chemical mutagens include, but are not limited to, ethyl methanesulfonate and N-ethyl-N-nitrosourea. Eventually a strain may obtain genetic changes enabling it to grow better in SPEM/NPEM. These evolved strains may be better or worse colonizers than their WT parents.

In some cases, a mutagenesis screen may be conducted on microbes which are normally attracted towards PEM. Such a screen may identify mutant microbes which are not attracted to the PEM, and sequencing of the mutant microbes may identify genes and regulatory sequences involved in sensing PEM, as well as migrating or moving towards PEM.

Further described herein are engineered microbes which comprise modifications that alter chemoattraction to PEM. In some cases, the engineered microbe may be produced by replicating a PEM chemoattraction related mutation discovered from a mutagenesis screen as described above. In some cases, the engineered microbe may be produced by altering regulation of a gene known to be involved in nutrient sensing or mobility. In certain cases, an engineered microbe may have improved chemoattraction for a component of PEM. In various cases, an engineered microbe may have decreased chemoattraction for a component of PEM.

### Plant Species

The methods and microbes described herein can be used with exudate from any of a variety of plants, for example, such as plants in the genera Hordeum, Oryza, Zea, and Triticeae. Other non-limiting examples of suitable plants include mosses, lichens, and algae.

In some cases, the plants have economic, social, and/or environmental value, such as food crops, fiber crops, oil crops, plants in the forestry or pulp and paper industries, feedstock for biofuel production, and/or ornamental plants. In some examples, plants may be used to produce economically valuable products such as a grain, a flour, a starch, a syrup, a meal, an oil, a film, a packaging, a nutraceutical product, a pulp, an animal feed, a fish fodder, a bulk material for industrial chemicals, a cereal product, a processed human-food product, a sugar, an alcohol, and/or a protein. Non-limiting examples of crop plants include maize, rice, wheat, barley, sorghum, millet, oats, rye, triticale, buckwheat, sweet corn, sugar cane, onions, tomatoes, strawberries, and asparagus.

In some examples, plants that may be obtained or improved using the methods and/or compositions disclosed herein may include plants that are important or interesting for agriculture, horticulture, biomass for the production of biofuel molecules and other chemicals, and/or forestry. Some examples of these plants may include pineapple, banana, coconut, lily, grass pea, alfalfa, tomatillo, melon, chickpea, chicory, clover, kale, lentil, soybean, tobacco, potato, sweet potato, radish, cabbage, rape, apple, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum bicolor (sorghum, sudan), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp. Sorghum spp., Miscanthus spp., Saccharum spp., Erianthus spp., Populus spp., Secale cereale (rye), Salix spp. (willow), Eucalyptus spp. (eucalyptus), Triticosecale spp. (triticum-25 wheat×rye), Bamboo, Carthamus tinctorius (safflower), Jatropha curcas (jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, Brussels sprout), Camellia sinensis (tea), Fragaria ananassa (strawberry), Theobroma cacao (cocoa), Coffea arabica (coffee), Vitis vinifera (grape), Ananas comosus (pineapple), Capsicum annum (hot and sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Colchicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Hordeum vulgare (barley), and Lolium spp. (rye).

In some examples, a monocotyledonous plant may be used, including those belonging to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In some examples, the monocotyledonous plant can be selected from the group consisting of a maize, rice, wheat, barley, and sugarcane.

In some examples, a dicotyledonous plant may be used, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumbaginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In some examples, the dicotyledonous plant can be selected from the group consisting of cotton, soybean, pepper, and tomato.

In some cases, the plant to be improved may not be readily amenable to experimental conditions. For example, a crop plant may take too long to grow enough to practically assess an improved trait serially over multiple iterations. Accordingly, a first plant from which bacteria are initially isolated, and/or the plurality of plants to which genetically manipulated bacteria are applied may be a model plant, such as a plant more amenable to evaluation under desired conditions. Non-limiting examples of model plants include Setaria, Brachypodium, and Arabidopsis. Ability of microbes (e.g., bacteria) isolated according to a method of the disclosure using a model plant may then be applied to a plant of another type (e.g., a crop plant) to confirm conferral of the improved trait.

Traits that may be improved by the methods disclosed herein include any observable characteristic of the plant, including, for example, growth rate, height, weight, color, taste, smell, changes in the production of one or more compounds by the plant (including, for example, metabolites, proteins, drugs, carbohydrates, oils, and any other compounds). Selecting plants based on genotypic information is also envisaged (for example, including the pattern of plant gene expression in response to the bacteria or identifying the presence of genetic markers, such as those associated with increased nitrogen fixation). Plants may also be selected based on the absence, suppression, or inhibition of a certain feature or trait (such as an undesirable feature or trait) as opposed to the presence of a certain feature or trait (such as a desirable feature or trait).

Plant productivity can refer generally to any aspect of growth or development of a plant that can be a reason for which the plant may be grown. In some cases, for food crops, which can include grains or vegetables, plant productivity can refer to the yield of grain or fruit which may be harvested from a particular crop. As used herein, improved plant productivity may refer broadly to improvements in a yield, for example, a yield of grain, fruit, flowers, or other plant parts which may be harvested for a purpose. In some cases, improved plant productivity may refer broadly to improvements in growth of plant parts, which may include stems, leaves, and roots. In some cases, improved plant productivity may refer broadly to improvements in promotion of plant growth, maintenance of high chlorophyll content in leaves, increasing fruit or seed numbers, increasing fruit or seed unit weight, and/or reducing NO₂ emission due to reduced nitrogen fertilizer usage. In some cases, improved plant productivity may also refer to similar improvements of the growth and development of plants.

Microbes in and around food crops can influence a trait of those crops. In some cases, a plant trait that may be influenced by microbes may include: yield (e.g., grain production, biomass generation, fruit development, flower set); nutrition (e.g., nitrogen, phosphorus, potassium, iron, micronutrient acquisition); abiotic stress management (e.g., drought tolerance, salt tolerance, heat tolerance); and biotic stress management (e.g., pest, weeds, insects, fungi, and bacteria). Strategies for altering a crop trait can include in some instances: increasing key metabolite concentrations; changing temporal dynamics of microbe influence on key metabolites; linking microbial metabolite production/degradation to new environmental cues; reducing negative metabolites; and improving the balance of metabolites or underlying proteins.

In some cases, a control sequence can refer to a sequence which can be an operator, promoter, silencer, or terminator. In some embodiments, native or endogenous control sequences of genes of the present disclosure can be replaced with one or more intrageneric control sequences.

In some instances, introduced may refer to introduction using modern biotechnology, and may be not a naturally occurring introduction. In some embodiments, the bacteria of the present disclosure may have been modified such that they may not be naturally occurring bacteria.

In some embodiments, the bacteria of the present disclosure can be present in the plant in an amount of at least 10³ cfu, 10⁴ cfu, 10⁵ cfu, 10⁶ cfu, 10⁷ cfu, 10⁸ cfu, 10⁹ cfu, 10¹⁰ cfu, 10¹¹ cfu, or 10¹² cfu per gram of fresh weight or dry weight of the plant. In some embodiments, the bacteria of the present disclosure can be present in the plant in an amount of at least about 10³ cfu, about 10⁴ cfu, about 10⁵ cfu, about 10⁶ cfu, about 10⁷ cfu, about 10⁸ cfu, about 10⁹ cfu, about 10¹⁰ cfu, about 10¹¹ cfu, or about 10¹² cfu per gram of fresh weight or dry weight of the plant. In some embodiments, the bacteria of the present disclosure can be present in the plant in an amount of at least 10³ to 10⁹, 10³ to 10⁷, 10³ to 10⁵, 10⁵ to 10⁹, 10⁵ to 10⁷, 10⁶ to 10¹⁰, 10⁶ to 10⁷ cfu per gram of fresh weight or dry weight of the plant.

Fertilizers and/or exogenous nitrogen of the present disclosure may comprise the following nitrogen-containing molecules: ammonium, nitrate, nitrite, ammonia, glutamine, etc. Nitrogen sources of the present disclosure may include anhydrous ammonia, ammonia sulfate, urea, diammonium phosphate, urea-form, monoammonium phosphate, ammonium nitrate, nitrogen solutions, calcium nitrate, potassium nitrate, sodium nitrate, etc.

In some cases, exogenous nitrogen refers to non-atmospheric nitrogen readily available in the soil, field, or growth medium that is present under non-nitrogen limiting conditions, including ammonia, ammonium, nitrate, nitrite, urea, uric acid, ammonium acids, etc.

In some cases, non-nitrogen limiting conditions can refer to non-atmospheric nitrogen available in the soil, field, or media at concentrations which may be greater than about 4 mM, 3 mM, 2 mM, 1 mM, 0.5 mM, 0.25 mM, or 0.05 mM nitrogen.

In some embodiments, the nitrogen fixation and assimilation genetic regulatory network can comprise polynucleotides which can encode genes and/or non-coding sequences that can direct, modulate, and/or regulate microbial nitrogen fixation and/or assimilation, and/or can occasionally comprise a polynucleotide sequence of the nif cluster (e.g., nifA, nifB, nifC, . . . nifZ), a polynucleotide encoding nitrogen regulatory protein C, a polynucleotide encoding nitrogen regulatory protein B, a polynucleotide sequence of the gln cluster (e.g., glnA and glnD), draT, and/or ammonia transporters/permeases. In some cases, the Nif cluster may comprise NifB, NifH, NifD, NifK, NifE, NifN, NifX, hesa, and NifV. In some cases, the Nif cluster may comprise a subset of NifB, NifH, NifD, NifK, NifE, NifN, NifX, hesa, and NifV.

In some embodiments, fertilizer of the present disclosure can comprise at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% nitrogen by weight.

In some embodiments, fertilizer of the present disclosure can comprise at least about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 8′7%, about 88%, about 89%, about 90%, about 91%, about 92%, about 93%, about 94%, about 95%, about 96%, about 97%, about 98%, or about 99% nitrogen by weight.

In some embodiments, fertilizer of the present disclosure may comprise about 5% to 50%, about 5% to 75%, about 10% to 50%, about 10% to 75%, about 15% to 50%, about 15% to 75%, about 20% to 50%, about 20% to 75%, about 25% to 50%, about 25% to 75%, about 30% to 50%, about 30% to 75%, about 35% to 50%, about 35% to 75%, about 40% to 50%, about 40% to 75%, about 45% to 50%, about 45% to 75%, or about 50% to 75% nitrogen by weight.

In some embodiments, the increase of nitrogen fixation and/or the production of 1% or more of the nitrogen in the plant can be measured relative to plants, which may be control plants, which possibly have not been exposed to certain bacteria of the present disclosure. In some cases, some or all increases or decreases in bacteria are measured relative to bacteria which can be control bacteria. In some cases, some or all increases or decreases in plants can be measured relative to plants which can be control plants.

In some instances, a constitutive promoter can be a promoter which can be active under most conditions and/or during most development stages. There can be several advantages to using constitutive promoters in expression vectors which may be used in biotechnology. Such advantages can include in some cases: a high level of production of proteins which may be used to select transgenic cells or organisms; a high level of expression of reporter proteins and/or scorable markers, which may allow for easy detection and/or quantification; a high level of production of a transcription factor that can be part of a regulatory transcription system; production of compounds that can require ubiquitous activity in the organism; and/or production of compounds that can be required during any stage of development, multiple stages of development, or all stages of development. Non-limiting exemplary constitutive promoters can include antibiotic resistance gene promoters such as the tetracycline resistance gene promoter.

In some cases, a non-constitutive promoter can be a promoter which may be active under certain conditions, in certain types of cells, and/or during certain development stages. For example, In some cases, these can include tissue specific, tissue preferred, cell type specific, cell type preferred, or inducible promoters. In some cases, promoters under developmental control may include non-constitutive promoters. Examples of promoters under developmental control can include promoters that may preferentially initiate transcription in certain tissues.

As used herein, an inducible or repressible promoter can be a promoter which may be under chemical or environmental factors control. Examples of environmental conditions that may affect transcription by inducible promoters can include anaerobic conditions, certain chemicals, the presence of light, acidic and/or basic conditions, etc.

As used herein, a tissue specific promoter can be a promoter that can initiates transcription in certain tissues, perhaps only in certain tissues. Unlike constitutive expression of genes, tissue-specific expression can be the result of up to several interacting levels of gene regulation. In the context of plant associated microbes, a tissue specific promoter may be a promoter which regulates expression of a gene (e.g., a linked gene) depending on the plant tissue with which the microbe is associated. For example, a tissue specific promoter may cause a gene to be expressed when the microbe is colonizing a leaf of a plant but not when colonizing a root.

In some cases, operably linked can refer to the association of nucleic acid sequences which may be on a single nucleic acid fragment such that the function of one may be regulated by the other. For example, in some instances a promoter may be operably linked with a coding sequence when it can be capable of at least sometimes regulating the expression of that coding sequence. Coding sequences can be operably linked to regulatory sequences, e.g., in a sense or antisense orientation. In another nonlimiting example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

Traits which can be targeted for regulation by the methods described herein can include nitrogen fixation, nitrogen excretion, desiccation tolerance, oxygen sensitivity, and other traits.

One trait that may be targeted for regulation by the methods described herein is nitrogen fixation. Nitrogen fertilizer is the largest operational expense on a farm and the biggest driver of higher yields in row crops like corn and wheat. Described herein are microbial products that can deliver renewable forms of nitrogen in non-leguminous crops. While some endophytes have the genetics for fixing nitrogen in pure culture, generally, the fundamental technical challenge is that wild-type endophytes of cereals and grasses stop fixing nitrogen in fertilized fields. The application of chemical fertilizers and residual nitrogen levels in field soils signal the microbe to shut down the biochemical pathway for nitrogen fixation.

Changes to the transcriptional and post-translational levels of nitrogen fixation regulatory network are required to develop a microbe capable of fixing and transferring nitrogen to corn in the presence of fertilizer. To that end, described herein is Host-Microbe Evolution (HoME) technology to precisely evolve regulatory networks and elicit novel phenotypes. Also described herein are unique, proprietary libraries of nitrogen-fixing endophytes isolated from corn, paired with extensive omics data surrounding the interaction of microbes and host plant under different environmental conditions like nitrogen stress and excess. This technology enables precision evolution of the genetic regulatory network of endophytes to produce microbes that actively fix nitrogen even in the presence of fertilizer in the field. Also described herein are evaluations of the technical potential of evolving microbes that colonize corn root tissues and produce nitrogen for fertilized plants and evaluations of the compatibility of endophytes with standard formulation practices and diverse soils to determine feasibility of integrating the microbes into modern nitrogen management strategies.

In order to utilize elemental nitrogen (N) for chemical synthesis, life forms combine nitrogen gas (N2) available in the atmosphere with hydrogen in a process known as nitrogen fixation. Because of the energy-intensive nature of biological nitrogen fixation, diazotrophs (bacteria and archaea that fix atmospheric nitrogen gas) have evolved sophisticated and tight regulation of the nif gene cluster in response to environmental oxygen and available nitrogen. Nif genes encode enzymes involved in nitrogen fixation (such as the nitrogenase complex) and proteins that regulate nitrogen fixation. Shamseldin (2013. Global J. Biotechnol. Biochem. 8(4):84-94) discloses detailed descriptions of nif genes and their products, and is incorporated herein by reference.

In Proteobacteria, regulation of nitrogen fixation centers around the am-dependent enhancer-binding protein NifA, the positive transcriptional regulator of the nif cluster. Intracellular levels of active NifA are controlled by two key factors: transcription of the nifLA operon, and inhibition of NifA activity by protein-protein interaction with NifL. Both of these processes are responsive to intracellular glutamine levels via the PII protein signaling cascade. This cascade is mediated by GlnD, which directly senses glutamine and catalyzes the uridylylation or deuridylylation of two PII regulatory proteins—GlnB and GlnK—in response the absence or presence, respectively, of bound glutamine. Under conditions of nitrogen excess, unmodified GlnB signals the deactivation of the nifLA promoter. However, under conditions of nitrogen limitation, GlnB is post-translationally modified, which inhibits its activity and leads to transcription of the nifLA operon. In this way, nifLA transcription is tightly controlled in response to environmental nitrogen via the PII protein signaling cascade. On the post-translational level of NifA regulation, GlnK inhibits the NifL/NifA interaction in a matter dependent on the overall level of free GlnK within the cell.

NifA is transcribed from the nifLA operon, whose promoter is activated by phosphorylated NtrC, another am-dependent regulator. The phosphorylation state of NtrC is mediated by the histidine kinase NtrB, which interacts with deuridylylated GlnB but not uridylylated GlnB. Under conditions of nitrogen excess, a high intracellular level of glutamine leads to deuridylylation of GlnB, which then interacts with NtrB to deactivate its phosphorylation activity and activate its phosphatase activity, resulting in dephosphorylation of NtrC and the deactivation of the nifLA promoter. However, under conditions of nitrogen limitation, a low level of intracellular glutamine results in uridylylation of GlnB, which inhibits its interaction with NtrB and allows the phosphorylation of NtrC and transcription of the nifLA operon. In this way, nifLA expression is tightly controlled in response to environmental nitrogen via the PII protein signaling cascade. nifA, ntrB, ntrC, and glnB, are all genes that can be mutated in the methods described herein. These processes may also be responsive to intracellular levels of ammonia, urea, or nitrates.

The activity of NifA is also regulated post-translationally in response to environmental nitrogen, most typically through NifL-mediated inhibition of NifA activity. In general, the interaction of NifL and NifA is influenced by the PII protein signaling cascade via GlnK, although the nature of the interactions between GlnK and NifL/NifA varies significantly between diazotrophs. In Klebsiella pneumoniae, both forms of GlnK inhibit the NifL/NifA interaction, and the interaction between GlnK and NifL/NifA is determined by the overall level of free GlnK within the cell. Under nitrogen-excess conditions, deuridylylated GlnK interacts with the ammonium transporter AmtB, which serves to both block ammonium uptake by AmtB and sequester GlnK to the membrane, allowing inhibition of NifA by NifL. On the other hand, in Azotobacter vinelandii, interaction with deuridylylated GlnK is required for the NifL/NifA interaction and NifA inhibition, while uridylylation of GlnK inhibits its interaction with NifL. In diazotrophs lacking the nifL gene, there is evidence that NifA activity is inhibited directly by interaction with the deuridylylated forms of both GlnK and GlnB under nitrogen-excess conditions. In some bacteria the Nif cluster may be regulated by glnR, and further In some cases, this may comprise negative regulation. Regardless of the mechanism, post-translational inhibition of NifA is an important regulator of the nif cluster in most known diazotrophs. Additionally, nifL, amtB, glnK, and glnR are genes that can have their expression altered in the methods described herein.

In addition to regulating the transcription of the nif gene cluster, many diazotrophs have evolved a mechanism for the direct post-translational modification and inhibition of the nitrogenase enzyme itself, known as nitrogenase shutoff. This is mediated by ADP-ribosylation of the Fe protein (NifH) under nitrogen-excess conditions, which disrupts its interaction with the MoFe protein complex (NifDK) and abolishes nitrogenase activity. DraT catalyzes the ADP-ribosylation of the Fe protein and shutoff of nitrogenase, while DraG catalyzes the removal of ADP-ribose and reactivation of nitrogenase. As with nifLA transcription and NifA inhibition, nitrogenase shutoff is also regulated via the PII protein signaling cascade. Under nitrogen-excess conditions, deuridylylated GlnB interacts with and activates DraT, while deuridylylated GlnK interacts with both DraG and AmtB to form a complex, sequestering DraG to the membrane. Under nitrogen-limiting conditions, the uridylylated forms of GlnB and GlnK do not interact with DraT and DraG, respectively, leading to the inactivation of DraT and the diffusion of DraG to the Fe protein, where it removes the ADP-ribose and activates nitrogenase. The methods described herein also contemplate altering expression via manipulation of the nifH, nifD, nifK, and draT genes.

Although some endophytes have the ability to fix nitrogen in vitro, often the genetics are silenced in the field by high levels of exogenous chemical fertilizers. One can decouple the sensing of exogenous nitrogen from expression of the nitrogenase enzyme to facilitate field-based nitrogen fixation. Improving the integral of nitrogenase activity across time further serves to augment the production of nitrogen for utilization by the crop. Specific targets for altering expression to facilitate field-based nitrogen fixation using the methods described herein include one or more genes selected from the group consisting of nifA, nifL, ntrB, ntrC, glnA, glnB, glnK, draT, amtB, glnD, glnE, nifJ, nifH, nifD, nifK, nifY, nifE, nifN, nifU, nifS, nifV, nifW, nifZ, nifM, nifF, nifB, and nifQ.

An additional target for altering expression to facilitate field-based nitrogen fixation using the methods described herein is the NifA protein. The NifA protein is typically the activator for expression of nitrogen fixation genes. Increasing the production of NifA (either constitutively or during high ammonia condition) circumvents the native ammonia-sensing pathway. In addition, reducing the production of NifL proteins, an inhibitor of NifA, also leads to an increased level of freely active NifA. In addition, increasing the transcription level of the nifAL operon (either constitutively or during high ammonia condition) also leads to an overall higher level of NifA proteins. Elevated level of nifAL expression is achieved by altering the promoter itself or by reducing the expression of NtrB (part of ntrB and ntrC signaling cascade that originally would result in the shutoff of nifAL operon during high nitrogen condition). High level of NifA achieved by these or any other methods described herein increases the nitrogen fixation activity of the endophytes.

Another target for altering expression to facilitate field-based nitrogen fixation using the methods described herein is the GlnD/GlnB/GlnK PII signaling cascade. The intracellular glutamine level is sensed through the GlnD/GlnB/GlnK PII signaling cascade. Active site mutations in GlnD that abolish the uridylyl-removing activity of GlnD disrupt the nitrogen-sensing cascade. In addition, reduction of the GlnB concentration short circuits the glutamine-sensing cascade. These mutations “trick” the cells into perceiving a nitrogen-limited state, thereby increasing the nitrogen fixation level activity. These processes may also be responsive to intracellular levels of ammonia, urea or nitrates.

The amtB protein can be a target for altering expression to facilitate field-based nitrogen fixation using the methods described herein. Ammonia uptake from the environment can be reduced by decreasing the expression level of amtB protein. Without intracellular ammonia, the endophyte is not able to sense the high level of ammonia, preventing the down-regulation of nitrogen fixation genes. Any ammonia that manages to get into the intracellular compartment is converted into glutamine. Intracellular glutamine level is the major currency of nitrogen sensing. Decreasing the intracellular glutamine level prevents the cells from sensing high ammonium levels in the environment. This effect can be achieved by increasing the expression level of glutaminase, an enzyme that converts glutamine into glutamate. In addition, intracellular glutamine can also be reduced by decreasing glutamine synthase (an enzyme that converts ammonia into glutamine). In diazotrophs, fixed ammonia is quickly assimilated into glutamine and glutamate to be used for cellular processes. Disruptions to ammonia assimilation may enable diversion of fixed nitrogen to be exported from the cell as ammonia. The fixed ammonia is predominantly assimilated into glutamine by glutamine synthetase (GS), encoded by glnA, and subsequently into glutamine by glutamine oxoglutarate aminotransferase (GOGAT). In some examples, glnS encodes a glutamine synthetase. GS is regulated post-translationally by GS adenylyl transferase (GlnE), a bi-functional enzyme encoded by glnE that catalyzes both the adenylylation and de-adenylylation of GS through activity of its adenylyl-transferase (AT) and adenylyl-removing (AR) domains, respectively. Under nitrogen limiting conditions, glnA is expressed, and GlnE's AR domain de-adynylylates GS, allowing it to be active. Under conditions of nitrogen excess, glnA expression is turned off, and GlnE's AT domain is activated allosterically by glutamine, causing the adenylylation and deactivation of GS.

Furthermore, the draT gene may also be a target for altering expression to facilitate field-based nitrogen fixation using the methods described herein. Once nitrogen fixing enzymes are produced by the cell, nitrogenase shut-off represents another level in which cell downregulates fixation activity in high nitrogen condition. This shut-off may be removed by decreasing the expression level of DraT.

Methods for altering gene expression of microbes can affect microbes at the transcriptional, translational, or post-translational levels. The transcriptional level includes changes at the promoter (such as changing sigma factor affinity or binding sites for transcription factors, including deletion of all or a portion of the promoter) or changing transcription terminators and attenuators. The translational level includes changes at the ribosome binding sites and changing mRNA degradation signals. The post-translational level includes mutating an enzyme's active site and changing protein-protein interactions.

Conversely, expression level of the genes described herein can be achieved by strengthening a promoter. To ensure high promoter activity during high nitrogen level condition (or any other condition), a transcription profile of the whole genome in a high nitrogen level condition may be obtained and active promoters with a desired transcription level can be chosen from that dataset to replace the weak promoter. An exudate which can strengthen a weak or strong promoter can be used.

Increasing the level of nitrogen fixation that occurs in a plant can lead to a reduction in the amount of chemical fertilizer needed for crop production and reduce greenhouse gas emissions (e.g., nitrous oxide).

### Generation of Bacterial Populations

### Isolation of Bacteria

Microbes useful in methods and compositions disclosed herein can be obtained by extracting microbes from surfaces or tissues of native plants. Microbes can be obtained by grinding seeds to isolate microbes. Microbes can be obtained by planting seeds in diverse soil samples and recovering microbes from tissues. Additionally, microbes can be obtained by inoculating plants with exogenous microbes and determining which microbes appear in plant tissues. Non-limiting examples of plant tissues may include a seed, seedling, leaf, cutting, plant, bulb, or tuber.

A method of obtaining microbes may be through the isolation of bacteria from soils. Bacteria may be collected from various soil types. In some example, the soil can be characterized by traits such as high or low fertility, levels of moisture, levels of minerals, and various cropping practices. For example, the soil may be involved in a crop rotation where different crops are planted in the same soil in successive planting seasons. The sequential growth of different crops on the same soil may prevent disproportionate depletion of certain minerals. The bacteria can be isolated from the plants growing in the selected soils. The seedling plants can be harvested at 2-6 weeks of growth. For example, at least 400 isolates can be collected in a round of harvest. Soil and plant types reveal the plant phenotype as well as the conditions, which allow for the downstream enrichment of certain phenotypes.

Microbes can be isolated from plant tissues to assess microbial traits. The parameters for processing tissue samples may be varied to isolate different types of associative microbes, such as rhizospheric bacteria, epiphytes, or endophytes. The isolates can be cultured in nitrogen-free media to enrich for bacteria that perform nitrogen fixation. Alternatively, microbes can be obtained from global strain banks.

In planta analytics are performed to assess microbial traits. In some embodiments, the plant tissue can be processed for screening by high throughput processing for DNA and RNA. Additionally, non-invasive measurements can be used to assess plant characteristics, such as colonization. Measurements on wild microbes can be obtained on a plant-by-plant basis. Measurements on wild microbes can also be obtained in the field using medium throughput methods. Measurements can be done successively over time. Model plant system can be used including, but not limited to, Setaria.

Microbes in a plant system can be screened via transcriptional profiling of a microbe in a plant system. Examples of screening through transcriptional profiling are using methods of quantitative polymerase chain reaction (qPCR), molecular barcodes for transcript detection, Next Generation Sequencing, and microbe tagging with fluorescent markers. Impact factors can be measured to assess colonization in the greenhouse including, but not limited to, microbiome, abiotic factors, soil conditions, oxygen, moisture, temperature, inoculum conditions, and root localization. Nitrogen fixation can be assessed in bacteria by measuring 15N gas/fertilizer (dilution) with IRMS or NanoSIMS as described herein NanoSIMS is high-resolution secondary ion mass spectrometry. The NanoSIMS technique is a way to investigate chemical activity from biological samples. The catalysis of reduction of oxidation reactions that drive the metabolism of microbes can be investigated at the cellular, subcellular, molecular and elemental level. NanoSIMS can provide high spatial resolution of greater than 0.1 μm. NanoSIMS can detect the use of isotope tracers such as ¹³C, ¹⁵N, and ¹⁸O. Therefore, NanoSIMS can be used to the chemical activity nitrogen in the cell.

Automated greenhouses can be used for in planta analytics. Plant metrics in response to microbial exposure include, but are not limited to, biomass, chloroplast analysis, CCD camera, and volumetric tomography measurements.

One way of enriching a microbe population is according to genotype. For example, a polymerase chain reaction (PCR) assay with a targeted primer or specific primer. Primers designed for the nifH gene can be used to identity diazotrophs because diazotrophs express the nifH gene in the process of nitrogen fixation. A microbial population can also be enriched via single-cell culture-independent approaches and chemotaxis-guided isolation approaches. Alternatively, targeted isolation of microbes can be performed by culturing the microbes on selection media. Premeditated approaches to enriching microbial populations for desired traits can be guided by bioinformatics data and are described herein.

Enriching for Microbes with Nitrogen Fixation Capabilities Using Bioinformatics

Bioinformatic tools can be used to identify and isolate plant growth promoting rhizobacteria (PGPRs), which are selected based on their ability to perform nitrogen fixation. Microbes with high nitrogen fixing ability can promote favorable traits in plants. Bioinformatic modes of analysis for the identification of PGPRs include, but are not limited to, genomics, metagenomics, targeted isolation, gene sequencing, transcriptome sequencing, and modeling.

Genomics analysis can be used to identify PGPRs and confirm the presence of mutations with methods of Next Generation Sequencing as described herein and microbe version control.

Metagenomics can be used to identify and isolate PGPR using a prediction algorithm for colonization. Metadata can also be used to identify the presence of an engineered strain in environmental and greenhouse samples.

Transcriptomic sequencing can be used to predict genotypes leading to PGPR phenotypes. Additionally, transcriptomic data is used to identify promoters for altering gene expression. Transcriptomic data can be analyzed in conjunction with the Whole Genome Sequence (WGS) to generate models of metabolism and gene regulatory networks.

### Domestication of Microbes

Microbes isolated from nature can undergo a domestication process wherein the microbes are converted to a form that is genetically trackable and identifiable. One way to domesticate a microbe is to engineer it with antibiotic resistance. The process of engineering antibiotic resistance can begin by determining the antibiotic sensitivity in the wild type microbial strain. If the bacteria are sensitive to the antibiotic, then the antibiotic can be a good candidate for antibiotic resistance engineering. Subsequently, an antibiotic resistant gene or a counterselectable suicide vector can be incorporated into the genome of a microbe using recombineering methods. A counterselectable suicide vector may consist of a deletion of the gene of interest, a selectable marker, and the counterselectable marker sacB. Counterselection can be used to exchange native microbial DNA sequences with antibiotic resistant genes. A medium throughput method can be used to evaluate multiple microbes simultaneously allowing for parallel domestication. Alternative methods of domestication include the use of homing nucleases to prevent the suicide vector sequences from looping out or from obtaining intervening vector sequences.

DNA vectors can be introduced into bacteria via several methods including electroporation and chemical transformations. A standard library of vectors can be used for transformations. An example of a method of gene editing is CRISPR preceded by Cas9 testing to ensure activity of Cas9 in the microbes.

### Non-Transgenic Engineering of Microbes

A microbial population with favorable traits can be obtained via directed evolution. Directed evolution is an approach wherein the process of natural selection is mimicked to evolve proteins or nucleic acids towards a user-defined goal. An example of directed evolution is when random mutations are introduced into a microbial population, the microbes with the most favorable traits are selected, and the growth of the selected microbes is continued. The most favorable traits in growth promoting rhizobacteria (PGPRs) may be in nitrogen fixation. The method of directed evolution may be iterative and adaptive based on the selection process after each iteration.

Plant growth promoting rhizobacteria (PGPRs) with high capability of nitrogen fixation can be generated. The evolution of PGPRs can be carried out via the introduction of genetic variation. Genetic variation can be introduced via polymerase chain reaction mutagenesis, oligonucleotide-directed mutagenesis, saturation mutagenesis, fragment shuffling mutagenesis, homologous recombination, CRISPR/Cas9 systems, chemical mutagenesis, and combinations thereof. These approaches can introduce random mutations into the microbial population. For example, mutants can be generated using synthetic DNA or RNA via oligonucleotide-directed mutagenesis. Mutants can be generated using tools contained on plasmids, which are later cured. Genes of interest can be identified using libraries from other species with improved traits including, but not limited to, improved PGPR properties, improved colonization of cereals, increased oxygen sensitivity, increased nitrogen fixation, and increased ammonia excretion. Intrageneric genes can be designed based on these libraries using software such as Geneious or Platypus design software. Mutations can be designed with the aid of machine learning. Mutations can be designed with the aid of a metabolic model. Automated design of the mutation can be done using a la Platypus and will guide RNAs for Cas-directed mutagenesis.

The intra-generic genes can be transferred into the host microbe. Additionally, reporter systems can also be transferred to the microbe. The reporter systems characterize promoters, determine the transformation success, screen mutants, and act as negative screening tools.

The microbes carrying the mutation can be cultured via serial passaging. A microbial colony contains a single variant of the microbe. Microbial colonies are screened with the aid of an automated colony picker and liquid handler. Mutants with gene duplication and increased copy number express a higher genotype of the desired trait.

### Selection of Plant Growth Promoting Microbes Based on Nitrogen Fixation

The microbial colonies can be screened using various assays to assess nitrogen fixation. One way to measure nitrogen fixation is via a single fermentative assay, which measures nitrogen excretion. An alternative method is the ARA with in-line sampling over time. ARA can be performed in high throughput plates of microtube arrays. ARA can be performed with live plants and plant tissues. The media formulation and media oxygen concentration can be varied in ARAs. Another method of screening microbial variants is by using biosensors. The use of NanoSIMS and Raman microspectroscopy can be used to investigate the activity of the microbes. In some cases, bacteria can also be cultured and expanded using methods of fermentation in bioreactors. The bioreactors are designed to improve robustness of bacteria growth and to decrease the sensitivity of bacteria to oxygen. Medium to high TP plate-based microfermentors are used to evaluate oxygen sensitivity, nutritional needs, nitrogen fixation, and nitrogen excretion. The bacteria can also be co-cultured with competitive or beneficial microbes to elucidate cryptic pathways. Flow cytometry can be used to screen for bacteria that produce high levels of nitrogen using chemical, colorimetric, or fluorescent indicators. The bacteria may be cultured in the presence or absence of a nitrogen source. For example, the bacteria may be cultured with glutamine, ammonia, urea, or nitrates.

In some cases, microbes may be screened in in planta assays, or in assays that mimic an in planta assay. In some cases, microbes may be grown with a plant in a field, with a plant in a greenhouse, with a plant in a hydroponic system, in media which has been exposed to plant roots, leaves, or stems (e.g., natural PEM), or in media which has been designed to mimic a plant environment (e.g., synthetic PEM). For example, the microbe may be grown in an in vitro media, which is constructed to mimic plant root exudates. PEM may become a proxy in which the microbe may be studied in vitro, but under conditions that more closely mimic the environment that the microbe may encounter in the field. In some cases, PEM may be generated by dipping seedlings or root tissue (e.g., corn root tissue) in aqueous solution. Plant roots can excrete carbon sources, amino acids, and other metabolites into their surroundings. Collecting the aqueous solution contacted with such plant tissue can allow the microbes to be grown in a substrate which may better mimic the environment the microbes will encounter in the field. As discussed above, plant exudates can be obtained, for example, by grinding root tissue in an aqueous solution to release metabolites into the aqueous solution for microbial growth.

Introducing a genetic variation may comprise insertion and/or deletion of one or more nucleotides at a target site, such as 1, 2, 3, 4, 5, 10, 25, 50, 100, 250, 500, or more nucleotides. The genetic variation introduced into one or more bacteria of the methods disclosed herein may be a knock-out mutation (e.g. deletion of a promoter, insertion or deletion to produce a premature stop codon, deletion of an entire gene), or it may be elimination or abolishment of activity of a protein domain (e.g. point mutation affecting an active site, or deletion of a portion of a gene encoding the relevant portion of the protein product), or it may alter or abolish a regulatory sequence of a target gene. One or more regulatory sequences may also be inserted, including heterologous regulatory sequences and regulatory sequences found within a genome of a bacterial species or genus corresponding to the bacteria into which the genetic variation is introduced. Moreover, regulatory sequences may be selected based on the expression level of a gene in a bacterial culture or within a plant tissue. The genetic variation may be a pre-determined genetic variation that is specifically introduced to a target site. The genetic variation may be a random mutation within the target site. The genetic variation may be an insertion or deletion of one or more nucleotides. In some cases, a plurality of different genetic variations (e.g., 2, 3, 4, 5, 10, or more) are introduced into one or more of the isolated bacteria before exposing the bacteria to plants for assessing trait improvement. The plurality of genetic variations can be any of the above types, the same or different types, and in any combination. In some cases, a plurality of different genetic variations are introduced serially, introducing a first genetic variation after a first isolation step, a second genetic variation after a second isolation step, and so forth so as to accumulate a plurality of genetic variations in bacteria imparting progressively improved traits on the associated plants.

A genetic variation may be referred to as a “mutation,” and a sequence or organism comprising a genetic variation may be referred to as a “genetic variant” or “mutant”. Genetic variations can have any number of effects, such as the increase or decrease of some biological activity, including gene expression, metabolism, and cell signaling. Genetic variations can be specifically introduced to a target site, or introduced randomly. A variety of molecular tools and methods are available for introducing genetic variation. For example, genetic variation can be introduced via polymerase chain reaction mutagenesis, oligonucleotide-directed mutagenesis, saturation mutagenesis, fragment shuffling mutagenesis, homologous recombination, recombineering, lambda red mediated recombination, CRISPR/Cas9 systems, chemical mutagenesis, and combinations thereof. Chemical methods of introducing genetic variation include exposure of DNA to a chemical mutagen, e.g., ethyl methanesulfonate (EMS), methyl methanesulfonate (MMS), N-nitrosourea (EN U), N-methyl-N-nitro-N′-nitrosoguanidine, 4-nitroquinoline N-oxide, diethyl sulfate, benzopyrene, cyclophosphamide, bleomycin, triethylmelamine, acrylamide monomer, nitrogen mustard, vincristine, diepoxyalkanes (for example, diepoxybutane), ICR-170, formaldehyde, procarbazine hydrochloride, ethylene oxide, dimethylnitrosamine, 7,12 dimethylbenz(a)anthracene, chlorambucil, hexamethylphosphoramide, bisulfan, and the like. Radiation mutation-inducing agents include ultraviolet radiation, y-irradiation, X-rays, and fast neutron bombardment. Genetic variation can also be introduced into a nucleic acid using, e.g., trimethylpsoralen with ultraviolet light. Random or targeted insertion of a mobile DNA element, e.g., a transposable element, is another suitable method for generating genetic variation. Genetic variations can be introduced into a nucleic acid during amplification in a cell-free in vitro system, e.g., using a polymerase chain reaction (PCR) technique such as error-prone PCR. Genetic variations can be introduced into a nucleic acid in vitro using DNA shuffling techniques (e.g., exon shuffling, domain swapping, and the like). Genetic variations can also be introduced into a nucleic acid as a result of a deficiency in a DNA repair enzyme in a cell, e.g., the presence in a cell of a mutant gene encoding a mutant DNA repair enzyme is expected to generate a high frequency of mutations (i.e., about 1 mutation/100 genes-1 mutation/10,000 genes) in the genome of the cell. Examples of genes encoding DNA repair enzymes include but are not limited to Mut H, Mut S, Mut L, and Mut U, and the homologs thereof in other species (e.g., MSH 1 6, PMS 1 2, MLH 1, GTBP, ERCC-1, and the like). Example descriptions of various methods for introducing genetic variations are provided in e.g., Stemple (2004) Nature 5:1-7; Chiang et al. (1993) PCR Methods Appl 2(3): 210-217; Stemmer (1994) Proc. Natl. Acad. Sci. USA 91:10747-10751; and U.S. Pat. Nos. 6,033,861, and 6,773,900.

Genetic variations introduced into microbes may be classified as transgenic, cisgenic, intragenomic, intrageneric, intergeneric, synthetic, evolved, rearranged, or SNPs.

Genetic variation may be introduced into numerous metabolic pathways within microbes to elicit improvements in the traits described above. Representative pathways include sulfur uptake pathways, glycogen biosynthesis, the glutamine regulation pathway, the molybdenum uptake pathway, the nitrogen fixation pathway, ammonia assimilation, ammonia excretion or secretion, nitrogen uptake, glutamine biosynthesis, annamox, phosphate solubilization, organic acid transport, organic acid production, agglutinins production, reactive oxygen radical scavenging genes, Indole Acetic Acid biosynthesis, trehalose biosynthesis, plant cell wall degrading enzymes or pathways, root attachment genes, exopolysaccharide secretion, glutamate synthase pathway, iron uptake pathways, siderophore pathway, chitinase pathway, ACC deaminase, glutathione biosynthesis, phosphorous signaling genes, quorum quenching pathway, cytochrome pathways, hemoglobin pathway, bacterial hemoglobin-like pathway, small RNA rsmZ, rhizobitoxine biosynthesis, lapA adhesion protein, AHL quorum sensing pathway, phenazine biosynthesis, cyclic lipopeptide biosynthesis, and antibiotic production.

CRISPR/Cas9 (Clustered regularly interspaced short palindromic repeats) /CRISPR-associated (Cas) systems can be used to introduce desired mutations. CRISPR/Cas9 provide bacteria and archaea with adaptive immunity against viruses and plasmids by using CRISPR RNAs (crRNAs) to guide the silencing of invading nucleic acids. The Cas9 protein (or functional equivalent and/or variant thereof, i.e., Cas9-like protein) naturally contains DNA endonuclease activity that depends on the association of the protein with two naturally occurring or synthetic RNA molecules called crRNA and tracrRNA (also called guide RNAs). In some cases, the two molecules are covalently linked to form a single molecule (also called a single guide RNA (“sgRNA”). Thus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence. If the Cas9 or Cas9-like protein retains its natural enzymatic function, it will cleave target DNA to create a double-stranded break, which can lead to genome alteration (i.e., editing: deletion, insertion (when a donor polynucleotide is present), replacement, etc.), thereby altering gene expression. Some variants of Cas9 (which variants are encompassed by the term Cas9-like) have been altered such that they have a decreased DNA cleaving activity (in some cases, they cleave a single strand instead of both strands of the target DNA, while in other cases, they have severely reduced to no DNA cleavage activity). Further exemplary descriptions of CRISPR systems for introducing genetic variation can be found in, e.g. U.S. Pat. No. 8,795,965.

As a cyclic amplification technique, polymerase chain reaction (PCR) mutagenesis uses mutagenic primers to introduce desired mutations. PCR is performed by cycles of denaturation, annealing, and extension. After amplification by PCR, selection of mutated DNA and removal of parental plasmid DNA can be accomplished by: 1) replacement of dCTP by hydroxymethylated-dCTP during PCR, followed by digestion with restriction enzymes to remove non-hydroxymethylated parent DNA only; 2) simultaneous mutagenesis of both an antibiotic resistance gene and the studied gene changing the plasmid to a different antibiotic resistance, the new antibiotic resistance facilitating the selection of the desired mutation thereafter; 3) after introducing a desired mutation, digestion of the parent methylated template DNA by restriction enzyme Dpnl which cleaves only methylated DNA , by which the mutagenized unmethylated chains are recovered; or 4) circularization of the mutated PCR products in an additional ligation reaction to increase the transformation efficiency of mutated DNA. Further description of exemplary methods can be found in e.g. U.S. Pat. Nos. 7,132,265, 6,713,285, 6,673,610, 6,391,548, 5,789,166, 5,780,270, 5,354,670, 5,071,743, and US20100267147.

Oligonucleotide-directed mutagenesis, also called site-directed mutagenesis, typically utilizes a synthetic DNA primer. This synthetic primer contains the desired mutation and is complementary to the template DNA around the mutation site so that it can hybridize with the DNA in the gene of interest. The mutation may be a single base change (a point mutation), multiple base changes, deletion, or insertion, or a combination of these. The single-strand primer is then extended using a DNA polymerase, which copies the rest of the gene. The gene thus copied contains the mutated site, and may then be introduced into a host cell as a vector and cloned. Finally, mutants can be selected by DNA sequencing to check that they contain the desired mutation.

Genetic variations can be introduced using error-prone PCR. In this technique the gene of interest is amplified using a DNA polymerase under conditions that are deficient in the fidelity of replication of sequence. The result is that the amplification products contain at least one error in the sequence. When a gene is amplified and the resulting product(s) of the reaction contain one or more alterations in sequence when compared to the template molecule, the resulting products are mutagenized as compared to the template. Another way of introducing random mutations is exposing cells to a chemical mutagen, such as nitrosoguanidine or ethyl methanesulfonate (Nestmann, Mutat Res 1975 June; 28(3):323-30), and the vector containing the gene is then isolated from the host.

Saturation mutagenesis is another form of random mutagenesis, in which one tries to generate all or nearly all possible mutations at a specific site, or narrow region of a gene. In a general sense, saturation mutagenesis is comprised of mutagenizing a complete set of mutagenic cassettes (wherein each cassette is, for example, 1-500 bases in length) in defined polynucleotide sequence to be mutagenized (wherein the sequence to be mutagenized is, for example, from 15 to 100,000 bases in length). Therefore, a group of mutations (e.g. ranging from 1 to 100 mutations) is introduced into each cassette to be mutagenized. A grouping of mutations to be introduced into one cassette can be different or the same from a second grouping of mutations to be introduced into a second cassette during the application of one round of saturation mutagenesis. Such groupings are exemplified by deletions, additions, groupings of particular codons, and groupings of particular nucleotide cassettes.

Fragment shuffling mutagenesis, also called DNA shuffling, is a way to rapidly propagate beneficial mutations. In an example of a shuffling process, DNase is used to fragment a set of parent genes into pieces of e.g. about 50-100 bp in length. This is then followed by a polymerase chain reaction (PCR) without primers—DNA fragments with sufficient overlapping homologous sequence will anneal to each other and are then be extended by DNA polymerase. Several rounds of this PCR extension are allowed to occur, after some of the DNA molecules reach the size of the parental genes. These genes can then be amplified with another PCR, this time with the addition of primers that are designed to complement the ends of the strands. The primers may have additional sequences added to their 5′ ends, such as sequences for restriction enzyme recognition sites needed for ligation into a cloning vector. Further examples of shuffling techniques are provided in US20050266541.

Homologous recombination mutagenesis involves recombination between an exogenous DNA fragment and the targeted polynucleotide sequence. After a double-stranded break occurs, sections of DNA around the 5′ ends of the break are cut away in a process called resection. In the strand invasion step that follows, an overhanging 3′ end of the broken DNA molecule then “invades” a similar or identical DNA molecule that is not broken. The method can be used to delete a gene, remove exons, add a gene, and introduce point mutations. Homologous recombination mutagenesis can be permanent or conditional. Typically, a recombination template is also provided. A recombination template may be a component of another vector, contained in a separate vector, or provided as a separate polynucleotide. In some embodiments, a recombination template is designed to serve as a template in homologous recombination, such as within or near a target sequence nicked or cleaved by a site-specific nuclease. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence. Non-limiting examples of site-directed nucleases useful in methods of homologous recombination include zinc finger nucleases, CRISPR nucleases, TALE nucleases, and meganuclease. For a further description of the use of such nucleases, see e.g. U.S. Pat. No. 8,795,965 and US20140301990.

Mutagens that create primarily point mutations and short deletions, insertions, transversions, and/or transitions, including chemical mutagens or radiation, may be used to create genetic variations. Mutagens include, but are not limited to, ethyl methanesulfonate, methylmethane sulfonate, N-ethyl-N-nitrosurea, triethylmelamine, N-methyl-N-nitrosourea, procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine, nitrosoguanidine, 2-aminopurine, 7,12 dimethyl-benz(a)anthracene, ethylene oxide, hexamethylphosphoramide, bisulfan, diepoxyalkanes (diepoxyoctane, diepoxybutane, and the like), 2-methoxy-6-chloro-9[3-(ethyl-2-chloro-ethyl)aminopropylamino]acridine dihydrochloride and formaldehyde.

Introducing genetic variation may be an incomplete process, such that some bacteria in a treated population of bacteria carry a desired mutation while others do not. In some cases, it is desirable to apply a selection pressure so as to enrich for bacteria carrying a desired genetic variation. Traditionally, selection for successful genetic variants involved selection for or against some functionality imparted or abolished by the genetic variation, such as in the case of inserting antibiotic resistance gene or abolishing a metabolic activity capable of converting a non-lethal compound into a lethal metabolite. It is also possible to apply a selection pressure based on a polynucleotide sequence itself, such that only a desired genetic variation need be introduced (e.g. without also requiring a selectable marker). In this case, the selection pressure can comprise cleaving genomes lacking the genetic variation introduced to a target site, such that selection is effectively directed against the reference sequence into which the genetic variation is sought to be introduced. Typically, cleavage occurs within 100 nucleotides of the target site (e.g. within 75, 50, 25, 10, or fewer nucleotides from the target site, including cleavage at or within the target site). Cleaving may be directed by a site-specific nuclease selected from the group consisting of a Zinc Finger nuclease, a CRISPR nuclease, a TALE nuclease (TALEN), or a meganuclease. Such a process is similar to processes for enhancing homologous recombination at a target site, except that no template for homologous recombination is provided. As a result, bacteria lacking the desired genetic variation are more likely to undergo cleavage that, left unrepaired, results in cell death. Bacteria surviving selection may then be isolated for use in exposing to plants for assessing conferral of an improved trait.

A CRISPR nuclease may be used as the site-specific nuclease to direct cleavage to a target site. An improved selection of mutated microbes can be obtained by using Cas9 to kill non-mutated cells. Plants are then inoculated with the mutated microbes to re-confirm symbiosis and create evolutionary pressure to select for efficient symbionts. Microbes can then be re-isolated from plant tissues. CRISPR nuclease systems employed for selection against non-variants can employ similar elements to those described above with respect to introducing genetic variation, except that no template for homologous recombination is provided. Cleavage directed to the target site thus enhances death of affected cells.

Other options for specifically inducing cleavage at a target site are available, such as zinc finger nucleases, TALE nuclease (TALEN) systems, and meganuclease. Zinc-finger nucleases (ZFNs) are artificial DNA endonucleases generated by fusing a zinc finger DNA binding domain to a DNA cleavage domain. ZFNs can be engineered to target desired DNA sequences and this enables zinc-finger nucleases to cleave unique target sequences. When introduced into a cell, ZFNs can be used to edit target DNA in the cell (e.g., the cell's genome) by inducing double stranded breaks. Transcription activator-like effector nucleases (TALENs) are artificial DNA endonucleases generated by fusing a TAL (Transcription activator-like) effector DNA binding domain to a DNA cleavage domain. TALENS can be quickly engineered to bind practically any desired DNA sequence and when introduced into a cell, TALENs can be used to edit target DNA in the cell (e.g., the cell's genome) by inducing double strand breaks. Meganucleases (homing endonuclease) are endodeoxyribonucleases characterized by a large recognition site (double-stranded DNA sequences of 12 to 40 base pairs. Meganucleases can be used to replace, eliminate or modify sequences in a highly targeted way. By modifying their recognition sequence through protein engineering, the targeted sequence can be changed. Meganucleases can be used to modify all genome types, whether bacterial, plant or animal and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII.

Methods of the present disclosure may be employed to introduce or improve one or more of a variety of desirable traits. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, resistance to nematode stress, resistance to a fungal pathogen, resistance to a bacterial pathogen, resistance to a viral pathogen, level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the improved traits) grown under identical conditions.

A preferred trait to be introduced or improved is nitrogen fixation, as described herein. In some cases, a plant resulting from the methods described herein exhibits a difference in the trait that is at least about 5% greater, for example, at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under the same conditions in the soil. In additional examples, a plant resulting from the methods described herein exhibits a difference in the trait that is at least about 5% greater, for example, at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under similar conditions in the soil.

The trait to be improved may be assessed under conditions including the application of one or more biotic or abiotic stressors. Examples of stressors include abiotic stresses (such as heat stress, salt stress, drought stress, cold stress, and low nutrient stress) and biotic stresses (such as nematode stress, insect herbivory stress, fungal pathogen stress, bacterial pathogen stress, and viral pathogen stress).

### Nitrogen Fixation

Some methods described herein can provide for the use of a natural or synthetic PEM to alter nitrogen fixation genes. Nitrogen fixation can be a process wherein the bacteria produce 1% or more of nitrogen in the plant (e.g., 2%, 5%, 10%, or more), which may represent a nitrogen-fixation capability of at least 2-fold as compared to the plant in the absence of the bacteria. The bacteria may produce the nitrogen in the presence of fertilizer supplemented with glutamine, urea, nitrates, or ammonia. Genetic variations can be any genetic variation described herein, including examples provided above, in any number and any combination. Genes altered can be selected from the group consisting of, but not limited to, nifA, nifL, ntrB, ntrC, glutamine synthetase, glnA, glnB, glnK, draT, amtB, glutaminase, glnD, glnE, nifJ, nifH, nifD, nifK, nifY, nifE, nifN, nifU, nifS, nifV, nifW, nifZ, nifM, nifF, nifB, and nifQ. Changes in expression can result in one or more of: increased expression or activity of nifA or glutaminase; decreased expression or activity of nifL, ntrB, glutamine synthetase, glnB, glnK, draT, amtB; decreased adenylyl-removing activity of GlnE; or decreased uridylyl-removing activity of GlnD.

The amount of nitrogen fixation that occurs in the plants described herein may be measured in several ways, for example, by an ARA. An ARA can be performed in vitro or in vivo. Evidence that a particular bacterium is providing fixed nitrogen to a plant can include: 1) total plant N significantly increases upon inoculation, for example, with a concomitant increase in N concentration in the plant; 2) nitrogen deficiency symptoms are relieved under N-limiting conditions upon inoculation (which may include an increase in dry matter); 3) N₂ fixation is documented through the use of an ¹⁵N approach (which can be isotope dilution experiments, ¹⁵N₂ reduction assays, or ¹⁵N natural abundance assays); 4) fixed N is incorporated into a plant protein or metabolite; and 5) all of these effects are not seen in non-inoculated plants or in plants inoculated with a mutant of the inoculum strain.

### Microbial Species

Microbes useful in the methods and compositions disclosed herein may be obtained from any source. In some cases, microbes may be bacteria, archaea, protozoa, or fungi. The microbes of this disclosure may be nitrogen fixing microbes, for example, a nitrogen fixing bacteria, nitrogen fixing archaea, nitrogen fixing fungi, nitrogen fixing yeast, or nitrogen fixing protozoa. Microbes useful in the methods and compositions disclosed herein may be spore forming microbes, for example, spore forming bacteria. In some cases, bacteria useful in the methods and compositions disclosed herein may be Gram positive bacteria or Gram negative bacteria. In some cases, the bacteria may be an endospore forming bacteria of the Firmicute phylum. In some cases, the bacteria may be a diazotroph. In certain cases, the bacteria may not be a diazotroph.

The methods and compositions of this disclosure may be used with an archaea, such as, for example, Methanothermobacter thermoautotrophicus.

In some cases, bacteria which may be useful include, but are not limited to, Agrobacterium radiobacter, Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus) Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus, Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus licheniformis, Bacillus maroccanus, Bacillus megaterium, Bacillus metiens, Bacillus mycoides, Bacillus natto, Bacillus nematocida, Bacillus nigrificans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus uniflagellatus, Bradyrhizobium japonicum, Brevibacillus brevis Brevibacillus laterosporus (formerly Bacillus laterosporus), Chromobacterium subtsugae, Delftia acidovorans, Lactobacillus acidophilus, Lysobacter antibioticus, Lysobacter enzymogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorum (formerly Erwinia carotovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luminescens, Xenorhabdus nematophila, Rhodococcus globerulus AQ719 (NRRL Accession No. B-21663), Bacillus sp. AQ175 (ATCC Accession No. 55608), Bacillus sp. AQ 177 (ATCC Accession No. 55609), Bacillus sp. AQ178 (ATCC Accession No. 53522), and Streptomyces sp. strain NRRL Accession No. B-30145. In some cases, the bacterium may be Azotobacter chroococcum, Methanosarcina barkeri, Klesiella pneumoniae, Azotobacter vinelandii, Rhodobacter spharoides, Rhodobacter capsulatus, Rhodobcter palustris, Rhodosporillum rubrum, Rhizobium leguminosarum, or Rhizobium etli.

In some cases, the bacterium may be a species of Clostridium, for example, Clostridium pasteurianum, Clostridium beijerinckii, Clostridium perfringens, Clostridium tetani, or Clostridium acetobutylicum.

In some cases, bacteria used with the methods and compositions of the present disclosure may be cyanobacteria. Examples of cyanobacterial genera include Anabaena (for example, Anagaena sp. PCC7120), Nostoc (for example, Nostoc punctiforme), or Synechocystis (for example, Synechocystis sp. PCC6803).

In some cases, bacteria used with the methods and compositions of the present disclosure may belong to the phylum Chlorobi, for example, Chlorobium tepidum.

In some cases, microbes used with the methods and compositions of the present disclosure may comprise a gene homologous to a known NifH gene. Sequences of known NifH genes may be found in, for example, the Zehr lab NifH database (wwwzehr.pmc.ucsc.edu/nifH_Database_Public/, Apr. 4, 2014) or the Buckley lab NifH database (www.css.cornell.edu/faculty/buckley/nifh.htm, and Gaby, John Christian, and Daniel H. Buckley. “A comprehensive aligned nifH gene database: a multipurpose tool for studies of nitrogen-fixing bacteria.” Database 2014 (2014): bau001.). In some cases, microbes used with the methods and compositions of the present disclosure may comprise a sequence which encodes a polypeptide with at least 60%, 70%, 80%, 85%, 90%, 95%, 96%, 96%, 98%, 99%, or more than 99% sequence identity to a sequence from the Zehr lab NifH database (wwwzehr.pmc.ucsc.edu/nifH_Database_Public/, Apr. 4, 2014). In some cases, microbes used with the methods and compositions of the present disclosure may comprise a sequence which encodes a polypeptide with at least 60%, 70%, 80%, 85%, 90%, 95%, 96%, 96%, 98%, 99% or more than 99% sequence identity to a sequence from the Buckley lab NifH database (Gaby, John Christian, and Daniel H. Buckley. “A comprehensive aligned nifH gene database: a multipurpose tool for studies of nitrogen-fixing bacteria.” Database 2014 (2014): bau001.).

Microbes useful in the methods and compositions disclosed herein can be obtained by extracting microbes from surfaces or tissues of native plants; grinding seeds to isolate microbes; planting seeds in diverse soil samples and recovering microbes from tissues; or inoculating plants with exogenous microbes and determining which microbes appear in plant tissues. Non-limiting examples of plant tissues include a seed, seedling, leaf, cutting, plant, bulb, or tuber. In some cases, bacteria are isolated from a seed. The parameters for processing samples may be varied to isolate different types of associative microbes, such as rhizospheric microbes, epiphytes, or endophytes. Bacteria may also be sourced from a repository, such as environmental strain collections, instead of initially isolating from a first plant. The microbes can be genotyped and phenotyped, via sequencing the genomes of isolated microbes; profiling the composition of communities in planta; characterizing the transcriptomic functionality of communities or isolated microbes; or screening microbial features using selective or phenotypic media (e.g., nitrogen fixation or phosphate solubilization phenotypes). Selected candidate strains or populations can be obtained via sequence data; phenotype data; plant data (e.g., genome, phenotype, and/or yield data); soil data (e.g., pH, N/P/K content, and/or bulk soil biotic communities); or any combination of these.

The bacteria and methods of producing bacteria described herein may apply to bacteria able to self-propagate efficiently on the leaf surface, root surface, or inside plant tissues without inducing a damaging plant defense reaction, or bacteria that are resistant to plant defense responses. The bacteria described herein may be isolated by culturing a plant tissue extract or leaf surface wash in a medium with no added nitrogen. However, the bacteria may be unculturable, that is, not known to be culturable or difficult to culture using standard methods. The bacteria described herein may be an endophyte or an epiphyte or a bacterium inhabiting the plant rhizosphere (rhizospheric bacteria). The bacteria obtained after repeating the steps of introducing genetic variation, exposure to a plurality of plants, and isolating bacteria from plants with an improved trait one or more times (e.g., 1, 2, 3, 4, 5, 10, 15, 25, or more times) may be endophytic, epiphytic, or rhizospheric. Endophytes are organisms that enter the interior of plants without causing disease symptoms or eliciting the formation of symbiotic structures, and are of agronomic interest because they can enhance plant growth and improve the nutrition of plants (e.g., through nitrogen fixation). The bacteria can be a seed-borne endophyte. Seed-borne endophytes include bacteria associated with or derived from the seed of a grass or plant, such as a seed-borne bacterial endophyte found in mature, dry, undamaged (e.g., no cracks, visible fungal infection, or prematurely germinated) seeds. The seed-borne bacterial endophyte can be associated with or derived from the surface of the seed; alternatively, or in addition, it can be associated with or derived from the interior seed compartment (e.g., of a surface-sterilized seed). In some cases, a seed-borne bacterial endophyte is capable of replicating within the plant tissue, for example, the interior of the seed. Also, in some cases, the seed-borne bacterial endophyte is capable of surviving desiccation.

The bacterial isolated according to methods of the disclosure, or used in methods or compositions of the disclosure, can comprise a plurality of different bacterial taxa in combination. By way of example, the bacteria may include Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium, and Halomonas), Firmicutes (such as Bacillus, Paenibacillus, Lactobacillus, Mycoplasma, and Acetabacterium), and Actinobacteria (such as Streptomyces, Rhodacoccus, Microbacterium, and Curtobacterium). The bacteria used in methods and compositions of this disclosure may include nitrogen fixing bacterial consortia of two or more species. In some cases, one or more bacterial species of the bacterial consortia may be capable of fixing nitrogen. In some cases, one or more species of the bacterial consortia may facilitate or enhance the ability of other bacteria to fix nitrogen. The bacteria which fix nitrogen and the bacteria which enhance the ability of other bacteria to fix nitrogen may be the same or different. In some examples, a bacterial strain may be able to fix nitrogen when in combination with a different bacterial strain, or in a certain bacterial consortia, but may be unable to fix nitrogen in a monoculture. Examples of bacterial genera which may be found in a nitrogen fixing bacterial consortia include, but are not limited to, Herbaspirillum, Azospirillum, Enterobacter, and Bacillus.

Bacteria that can be produced by the methods disclosed herein include Azotobacter sp., Bradyrhizobium sp., Klebsiella sp., and Sinorhizobium sp. In some cases, the bacteria may be selected from the group consisting of: Azotobacter vinelandii, Bradyrhizobium japonicum, Klebsiella pneumoniae, and Sinorhizobium meliloti. In some cases, the bacteria may be of the genus Enterobacter or Rahnella. In some cases, the bacteria may be of the genus Frankia or Clostridium. Examples of bacteria of the genus Clostridium include, but are not limited to, Clostridium acetobutilicum, Clostridium pasteurianum, Clostridium beijerinckii, Clostridium perfringens, and Clostridium tetani. In some cases, the bacteria may be of the genus Paenibacillus, for example, Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus macquariensis, Paenibacillus pabuli, Paenibacillus peoriae, or Paenibacillus polymyxa.

In some examples, bacteria isolated according to methods of the disclosure can be a member of one or more of the following taxa: Achromobacter, Acidithiobacillus, Acidovorax, Acidovoraz, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus Haloredivivus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Envinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Methylobacterium, Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera incertae sedis, Xanthomonas, and Zimmermannella.

The bacteria may be obtained from any general terrestrial environment, including its soils, plants, fungi, animals (including invertebrates), and other biota, including the sediments, water, and biota of lakes and rivers; from the marine environment, its biota and sediments (for example, sea water, marine muds, marine plants, marine invertebrates (for example, sponges), marine vertebrates (for example, fish)); the terrestrial and marine geosphere (regolith and rock, for example, crushed subterranean rocks, sand and clays); the cryosphere and its meltwater; the atmosphere (for example, filtered aerial dusts, cloud, and rain droplets); urban, industrial, and other man-made environments (for example, accumulated organic and mineral matter on concrete, roadside gutters, roof surfaces, and road surfaces).

The plant from which the bacteria are obtained may be a plant having one or more desirable traits, for example, a plant which naturally grows in a particular environment or under certain conditions of interest. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the bacteria may be collected from commercial crops grown in such environments, or more specifically from individual crop plants best displaying a trait of interest amongst a crop grown in any specific environment: for example, the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste, or smell. The bacteria may be collected from a plant of interest or any material occurring in the environment of interest, including fungi and other animal and plant biota, soil, water, sediments, and other elements of the environment as referred to previously.

The bacteria may be isolated from plant tissue. This isolation can occur from any appropriate tissue in the plant, including, for example, root, stem, leaves, and plant reproductive tissues. By way of example, conventional methods for isolation from plants typically include the sterile excision of the plant material of interest (e.g., root or stem lengths, leaves), surface sterilization with an appropriate solution (e.g., 2% sodium hypochlorite), after which the plant material is placed on nutrient medium for microbial growth. Alternatively, the surface-sterilized plant material can be crushed in a sterile liquid (usually water) and the liquid suspension, including small pieces of the crushed plant material spread over the surface of a suitable solid agar medium, or media, which may or may not be selective (e.g., contain only phytic acid as a source of phosphorus). This approach can be especially useful for bacteria which form isolated colonies and can be picked off individually to separate plates of nutrient medium, and further purified to a single species by well-known methods. Alternatively, the plant root or foliage samples may not be surface sterilized but washed gently thus including surface-dwelling epiphytic microbes in the isolation process, or the epiphytic microbes can be isolated separately, by imprinting and lifting off pieces of plant roots, stems, or leaves onto the surface of an agar medium and then isolating individual colonies as above. This approach can be especially useful for bacteria, for example. Alternatively, the roots may be processed without washing off small quantities of soil attached to the roots, thus including microbes that colonize the plant rhizosphere. Otherwise, soil adhering to the roots can be removed, diluted, and spread out onto agar of suitable selective and non-selective media to isolate individual colonies of rhizospheric bacteria.

Biologically pure cultures of Rahnella aquatilis and Enterobacter sacchari were deposited on Jul. 14, 2015 with the American Type Culture Collection (ATCC; an International Depositary Authority), Manassas, Va., USA, and assigned ATTC Patent Deposit Designation numbers PTA-122293 and PTA-122294, respectively. These deposits were made under the provisions of the Budapest Treaty on the International Recognition of the Deposit of Microorganisms for the Purpose of Patent Procedure and the Regulations (Budapest Treaty).

### Compositions

Compositions comprising bacteria or bacterial populations produced according to methods described herein and/or having characteristics as described herein can be in the form of a liquid, a foam, or a dry product. In some examples, a composition comprising bacterial populations may be in the form of a dry powder, a slurry of powder and water, or a flowable seed treatment.

The composition can be fabricated in bioreactors such as continuous stirred tank reactors, batch reactors, and on the farm. In some examples, compositions can be stored in a container, such as a jug or in mini bulk. In some examples, compositions may be stored within an object selected from the group consisting of a bottle, jar, ampule, package, vessel, bag, box, bin, envelope, carton, container, silo, shipping container, truck bed, and/or case.

Compositions may also be used to improve plant traits. In some examples, one or more compositions may be coated onto a seed. In some examples, one or more compositions may be coated onto a seedling. In some examples, one or more compositions may be coated onto a surface of a seed. In some examples, one or more compositions may be coated as a layer above a surface of a seed. In some examples, a composition that is coated onto a seed may be in liquid form, in dry product form, in foam form, in a form of a slurry of powder and water, or in a flowable seed treatment. In some examples, one or more compositions may be applied to a seed and/or seedling by spraying, immersing, coating, encapsulating, and/or dusting the seed and/or seedling with the one or more compositions. In some examples, multiple bacteria or bacterial populations can be coated onto a seed and/or a seedling of the plant. In some examples, at least two, at least three, at least four, at least five, at least six, at least seven, at least eight, at least nine, at least ten, or more than ten bacteria of a bacterial combination can be selected from one of the following genera: Acidovorax, Agrobacterium, Bacillus, Burkholderia, Chryseobacterium, Curtobacterium, Enterobacter, Escherichia, Methylobacterium, Paenibacillus, Pantoea, Pseudomonas, Ralstonia, Saccharibacillus, Sphingomonas, and Stenotrophomonas.

In some examples, at least two, at least three, at least four, at least five, at least six, at least seven, at least eight, at least nine, at least ten, or more than ten bacteria and bacterial populations of an endophytic combination are selected from one of the following families: Bacillaceae, Burkholderiaceae, Comamonadaceae, Enterobacteriaceae, Flavobacteriaceae, Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis, Lasiosphaeriaceae, Netriaceae, and Pleosporaceae.

In some examples, at least two, at least three, at least four, at least five, at least six, at least seven, at least eight, at least night, at least ten, or more than ten bacteria and bacterial populations of an endophytic combination are selected from one of the following families: Bacillaceae, Burkholderiaceae, Comamonadaceae, Enterobacteriaceae, Flavobacteriaceae, Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis, Lasiosphaeriaceae, Netriaceae, and Pleosporaceae.

Examples of compositions may include seed coatings for commercially important agricultural crops, for example, sorghum, canola, tomato, strawberry, barley, rice, maize, and wheat. Examples of compositions can also include seed coatings for corn, soybean, canola, sorghum, potato, rice, vegetables, cereals, and oilseeds. Seeds as provided herein can be genetically modified organisms (GMO), non-GMO, organic, or conventional. In some examples, compositions may be sprayed on the plant aerial parts, or applied to the roots by inserting into furrows in which the plant seeds are planted, watering to the soil, or dipping the roots in a suspension of the composition. In some examples, compositions may be dehydrated in a suitable manner that maintains cell viability and the ability to artificially inoculate and colonize host plants. The bacterial species may be present in compositions at a concentration of between 10⁸ to 10¹⁰ CFU/ml. In some examples, compositions may be supplemented with trace metal ions, such as molybdenum ions, iron ions, manganese ions, or combinations of these ions. The concentration of ions in examples of compositions as described herein may be from about 0.1 mM to about 50 mM. Some examples of compositions may also be formulated with a carrier, such as beta-glucan, carboxylmethyl cellulose (CMC), bacterial extracellular polymeric substance (EPS), sugar, animal milk, or other suitable carriers. In some examples, peat or planting materials can be used as a carrier, or biopolymers in which a composition is entrapped in the biopolymer can be used as a carrier.

The compositions comprising the bacterial populations described herein may be coated onto the surface of a seed. As such, compositions comprising a seed coated with one or more bacteria described herein are also contemplated. The seed coating can be formed by mixing the bacterial population with a porous, chemically inert granular carrier. Alternatively, the compositions may be inserted directly into the furrows into which the seed is planted or sprayed onto the plant leaves or applied by dipping the roots into a suspension of the composition. An effective amount of the composition can be used to populate the sub-soil region adjacent to the roots of the plant with viable bacterial growth, or populate the leaves of the plant with viable bacterial growth. In general, an effective amount is an amount sufficient to result in plants with improved traits (e.g., a desired level of nitrogen fixation).

Bacterial compositions described herein can be formulated using an agriculturally acceptable carrier. The formulation useful for these embodiments may include at least one member selected from the group consisting of a tackifier, a microbial stabilizer, a fungicide, an antibacterial agent, a preservative, a stabilizer, a surfactant, an anti-complex agent, an herbicide, a nematicide, an insecticide, a plant growth regulator, a fertilizer, a rodenticide, a desiccant, a bactericide, a nutrient, or any combination thereof. In some examples, compositions may be shelf-stable. For example, any of the compositions described herein can include an agriculturally acceptable carrier (e.g., one or more of a fertilizer such as a non-naturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally acceptable carrier in the seed coating. In any of the compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below.

In some cases, bacteria are mixed with an agriculturally acceptable carrier. The carrier can be a solid carrier or liquid carrier, and in various forms including microspheres, powders, emulsions and the like. The carrier may be any one or more of a number of carriers that confer a variety of properties, such as increased stability, wettability, or dispersibility. Wetting agents such as natural or synthetic surfactants, which can be nonionic or ionic surfactants, or a combination thereof can be included in the composition. Water-in-oil emulsions can also be used to formulate a composition that includes the isolated bacteria (see, for example, U.S. Patent No. 7,485,451). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the bacteria, such as barley, rice, or other biological materials such as seed, plant parts, sugar cane bagasse, hulls or stalks from grain processing, ground plant material or wood from building site refuse, sawdust or small fibers from recycling of paper, fabric, or wood.

For example, a fertilizer can be used to help promote the growth or provide nutrients to a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin. In one embodiment, the formulation can include a tackifier or adherent (referred to as an adhesive agent) to help bind other active agents to a substance (e.g., a surface of a seed). Such agents are useful for combining bacteria with carriers that can contain other compounds (e.g., control agents that are not biologic), to yield a coating composition. Such compositions help create coatings around the plant or seed to maintain contact between the microbe and other agents with the plant or plant part. In one embodiment, adhesives are selected from the group consisting of: alginate, gums, starches, lecithins, formononetin, polyvinyl alcohol, alkali formononetinate, hesperetin, polyvinyl acetate, cephalins, gum arabic, xanthan gum, mineral oil, polyethylene glycol (PEG), polyvinyl pyrrolidone (PVP), arabino-galactan, methyl cellulose, PEG 400, chitosan, polyacrylamide, polyacrylate, polyacrylonitrile, glycerol, triethylene glycol, vinyl acetate, gellan gum, polystyrene, polyvinyl, carboxymethyl cellulose, gum ghatti, and polyoxyethylene-polyoxybutylene block copolymers.

In some embodiments, the adhesives can be, e.g., a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arabics, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some examples, one or more of the adhesion agents, anti-fungal agents, growth regulation agents, and pesticides (e.g., insecticide) are non-naturally occurring compounds (e.g., in any combination). Additional examples of agriculturally acceptable carriers include dispersants (e.g., polyvinylpyrrolidone/vinyl acetate PVPIVA S-630), surfactants, binders, and filler agents.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant, which can include any compound or mixture of compounds that can be classified as a desiccant regardless of whether the compound or compounds are used in such concentrations that they in fact have a desiccating effect on a liquid inoculant. Such desiccants are ideally compatible with the bacterial population used, and may promote the ability of the microbial population to survive application on the seeds and to survive desiccation. Examples of suitable desiccants include one or more of trehalose, sucrose, glycerol, and methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, between about 10% to about 40%, between about 15% to about 35%, or between about 20% to about 30%. In some cases, it is advantageous for the formulation to contain agents such as a fungicide, an antibacterial agent, an herbicide, a nematicide, an insecticide, a plant growth regulator, a rodenticide, bactericide, or a nutrient. In some examples, agents may include protectants that provide protection against seed surface-borne pathogens. In some examples, protectants may provide some level of control of soil-borne pathogens. In some examples, protectants may be effective predominantly on a seed surface.

In some examples, a fungicide may include a compound or agent, whether chemical or biological, that can inhibit the growth of a fungus or kill a fungus. In some examples, a fungicide may include compounds that may be fungistatic or fungicidal. In some examples, fungicide can be a protectant, or agents that are effective predominantly on the seed surface, providing protection against seed surface-borne pathogens and providing some level of control of soil-borne pathogens. Non-limiting examples of protectant fungicides include captan, maneb, thiram, or fludioxonil.

In some examples, fungicide can be a systemic fungicide, which can be absorbed into the emerging seedling and inhibit or kill the fungus inside host plant tissues. Systemic fungicides used for seed treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some examples, fungicide can be a biological control agent, such as a bacterium or fungus. Such organisms may be parasitic to the pathogenic fungi, or secrete toxins or other substances which can kill or otherwise prevent the growth of fungi. Any type of fungicide, particularly ones that are commonly used on plants, can be used as a control agent in a seed composition.

In some examples, the seed coating composition may comprise a control agent which has antibacterial properties. In one embodiment, the control agent with antibacterial properties is selected from the compounds described herein elsewhere. In another embodiment, the compound is streptomycin, oxytetracycline, oxolinic acid, or gentamicin. Other examples of antibacterial compounds which can be used as part of a seed coating composition include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie).

In some examples, growth regulator is selected from the group consisting of: abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoaixins (e.g., glyceolline), and phytoalexin-inducing oligosaccharides (e.g., pectin, chitin, chitosan, polygalacuronic acid, and oligogalacturonic acid), and gibellerins. Such agents are ideally compatible with the agricultural seed or seedling onto which the formulation is applied (e.g., not deleterious to the growth or health of the plant). Furthermore, the agent is ideally one which does not cause safety concerns for human, animal, or industrial use (e.g., no safety issues, or the compound is sufficiently labile that the commodity plant product derived from the plant contains negligible amounts of the compound).

Some examples of nematode-antagonistic biocontrol agents include ARF18; 30 Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pasteuria usage, Brevibacillus laterosporus strain G4, Pseudomonas fluorescens, and Rhizobacteria.

Some examples of nutrients can be selected from the group consisting of a nitrogen fertilizer including, but not limited to urea, ammonium nitrate, ammonium sulfate, non-pressure nitrogen solutions, aqua ammonia, anhydrous ammonia, ammonium thiosulfate, sulfur-coated urea, urea-formaldehydes, IBDU, polymer-coated urea, calcium nitrate, ureaform, and methylene urea, phosphorous fertilizers such as diammonium phosphate, monoammonium phosphate, ammonium polyphosphate, concentrated superphosphate and triple superphosphate, and potassium fertilizers such as potassium chloride, potassium sulfate, potassium-magnesium sulfate, potassium nitrate. Such compositions can exist as free salts or ions within the seed coat composition. Alternatively, nutrients/fertilizers can be complexed or chelated to provide sustained release over time.

Some examples of rodenticides may include selected from the group of substances consisting of 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin, and zinc phosphide.

In the liquid form, for example, solutions or suspensions, bacterial populations can be mixed or suspended in water or in aqueous solutions. Suitable liquid diluents or carriers include water, aqueous solutions, petroleum distillates, or other liquid carriers.

Solid compositions can be prepared by dispersing the bacterial populations in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

### Application of Bacterial Populations on Crops

The composition of the bacteria or bacterial population described herein can be applied in furrow, in talc, or as seed treatment. The composition can be applied to a seed package in bulk, mini bulk, in a bag, or in talc.

The planter can plant the treated seed and grows the crop according to conventional ways, twin row, or ways that do not require tilling. The seeds can be distributed using a control hopper or an individual hopper. Seeds can also be distributed using pressurized air or manually. Seed placement can be performed using variable rate technologies. Additionally, application of the bacteria or bacterial population described herein may be applied using variable rate technologies. In some examples, the bacteria can be applied to seeds of corn, soybean, canola, sorghum, potato, rice, vegetables, cereals, pseudocereals, and oilseeds. Examples of cereals may include barley, fonio, oats, palmer's grass, turfgrass, rye, pearl millet, sorghum, spelt, teff, triticale, and wheat. Examples of pseudocereals may include breadnut, buckwheat, cattail, chia, flax, grain amaranth, hanza, quinoa, and sesame. In some examples, seeds can be genetically modified organisms (GMO), non-GMO, organic, or conventional.

Additives such as micro-fertilizer, PGR, herbicide, insecticide, and fungicide can be used additionally to treat the crops. Examples of additives include crop protectants such as insecticides, nematicides, fungicide, enhancement agents such as colorants, polymers, pelleting, priming, and disinfectants, and other agents such as inoculant, PGR, softener, and micronutrients. PGRs can be natural or synthetic plant hormones that affect root growth, flowering, or stem elongation. PGRs can include auxins, gibberellins, cytokinins, ethylene, and abscisic acid (ABA).

The composition can be applied in furrow in combination with liquid fertilizer. In some examples, the liquid fertilizer may be held in tanks. NPK fertilizers contain macronutrients of sodium, phosphorous, and potassium.

The composition may improve plant traits, such as promoting plant growth, maintaining high chlorophyll content in leaves, increasing fruit or seed numbers, and increasing fruit or seed unit weight. Examples of traits that may introduced or improved include: root biomass, root length, height, shoot length, leaf number, water use efficiency, overall biomass, yield, fruit size, grain size, photosynthesis rate, tolerance to drought, heat tolerance, salt tolerance, tolerance to low nitrogen stress, nitrogen use efficiency, resistance to nematode stress, resistance to a fungal pathogen, resistance to a bacterial pathogen, resistance to a viral pathogen, level of a metabolite, modulation in level of a metabolite, proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under identical conditions. In some examples, the desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under similar conditions.

An agronomic trait to a host plant may include, but is not limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without the seed treatment formulation

In some cases, plants are inoculated with bacteria or bacterial populations that are isolated from the same species of plant as the plant element of the inoculated plant. For example, an bacteria or bacterial population that is normally found in one variety of Zea mays (corn) is associated with a plant element of a plant of another variety of Zea mays that in its natural state lacks the bacteria and bacterial populations. In one embodiment, the bacteria and bacterial populations is derived from a plant of a related species of plant as the plant element of the inoculated plant. For example, an bacteria and bacterial populations that is normally found in Zea diploperennis Iltis et al., (diploperennial teosinte) is applied to a Zea mays (corn), or vice versa. In some cases, plants are inoculated with bacteria and bacterial populations that are heterologous to the plant element of the inoculated plant. In one embodiment, the bacteria and bacterial populations is derived from a plant of another species. For example, an bacteria and bacterial populations that is normally found in dicots is applied to a monocot plant (e.g., inoculating corn with a soybean-derived bacteria and bacterial populations), or vice versa. In other cases, the bacteria and bacterial populations to be inoculated onto a plant is derived from a related species of the plant that is being inoculated. In one embodiment, the bacteria and bacterial populations is derived from a related taxon, for example, from a related species. The plant of another species can be an agricultural plant. In another embodiment, the bacteria and bacterial populations is part of a designed composition inoculated into any host plant element.

In some examples, the bacteria or bacterial population is exogenous wherein the bacteria and bacterial population is isolated from a different plant than the inoculated plant. For example, in one embodiment, the bacteria or bacterial population can be isolated from a different plant of the same species as the inoculated plant. In some cases, the bacteria or bacterial population can be isolated from a species related to the inoculated plant.

In some examples, the bacteria and bacterial populations described herein are capable of moving from one tissue type to another. For example, the present disclosure's detection and isolation of bacteria and bacterial populations within the mature tissues of plants after coating on the exterior of a seed demonstrates their ability to move from seed exterior into the vegetative tissues of a maturing plant. Therefore, in one embodiment, the population of bacteria and bacterial populations is capable of moving from the seed exterior into the vegetative tissues of a plant. In one embodiment, the bacteria and bacterial populations that is coated onto the seed of a plant is capable, upon germination of the seed into a vegetative state, of localizing to a different tissue of the plant. For example, bacteria and bacterial populations can be capable of localizing to any one of the tissues in the plant, including: the root, adventitious root, seminal root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In one embodiment, the bacteria and bacterial populations is capable of localizing to the root and/or the root hair of the plant. In another embodiment, the bacteria and bacterial populations is capable of localizing to the photosynthetic tissues, for example, leaves and shoots of the plant. In other cases, the bacteria and bacterial populations is localized to the vascular tissues of the plant, for example, in the xylem and phloem. In still another embodiment, the bacteria and bacterial populations is capable of localizing to the reproductive tissues (flower, pollen, pistil, ovaries, stamen, or fruit) of the plant. In another embodiment, the bacteria and bacterial populations is capable of localizing to the root, shoots, leaves and reproductive tissues of the plant. In still another embodiment, the bacteria and bacterial populations colonizes a fruit or seed tissue of the plant. In still another embodiment, the bacteria and bacterial populations is able to colonize the plant such that it is present in the surface of the plant (i.e., its presence is detectably present on the plant exterior, or the episphere of the plant). In still other embodiments, the bacteria and bacterial populations is capable of localizing to substantially all, or all, tissues of the plant. In certain embodiments, the bacteria and bacterial populations is not localized to the root of a plant. In other cases, the bacteria and bacterial populations is not localized to the photosynthetic tissues of the plant.

The effectiveness of the compositions can also be assessed by measuring the relative maturity of the crop or the crop heating unit (CHU). For example, the bacterial population can be applied to corn, and corn growth can be assessed according to the relative maturity of the corn kernel or the time at which the corn kernel is at maximum weight. The crop heating unit (CHU) can also be used to predict the maturation of the corn crop. The CHU determines the amount of heat accumulation by measuring the daily maximum temperatures on crop growth.

In examples, bacteria may localize to any one of the tissues in the plant, including: the root, adventitious root, seminal root, root hair, shoot, leaf, flower, bud tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In another embodiment, the bacteria or bacterial population is capable of localizing to the photosynthetic tissues, for example, leaves and shoots of the plant. In other cases, the bacteria and bacterial populations is localized to the vascular tissues of the plant, for example, in the xylem and phloem. In another embodiment, the bacteria or bacterial population is capable of localizing to reproductive tissues (flower, pollen, pistil, ovaries, stamen, or fruit) of the plant. In another embodiment, the bacteria and bacterial populations is capable of localizing to the root, shoots, leaves, and reproductive tissues of the plant. In another embodiment, the bacteria or bacterial population colonizes a fruit or seed tissue of the plant. In still another embodiment, the bacteria or bacterial population is able to colonize the plant such that it is present in the surface of the plant. In another embodiment, the bacteria or bacterial population is capable of localizing to substantially all, or all, tissues of the plant. In certain embodiments, the bacteria or bacterial population is not localized to the root of a plant. In other cases, the bacteria and bacterial populations is not localized to the photosynthetic tissues of the plant.

The effectiveness of the bacterial compositions applied to crops can be assessed by measuring various features of crop growth including, but not limited to, planting rate, seeding vigor, root strength, drought tolerance, plant height, dry down, and test weight.

### EXAMPLES

The examples provided herein describe methods of bacterial isolation, bacterial and plant analysis, and plant trait improvement. The examples are for illustrative purposes only and are not to be construed as limiting in any way.

### Example 1: Analysis of Natural Plant Exudate

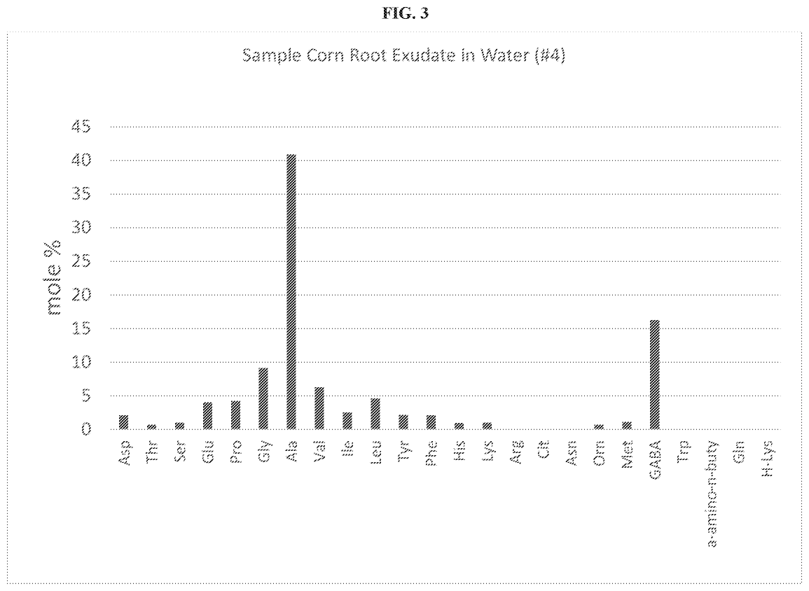

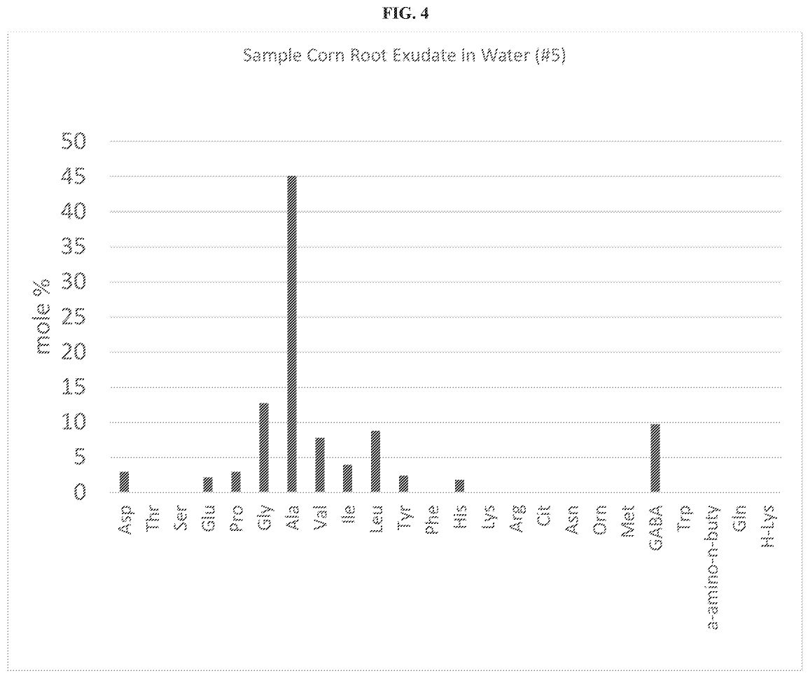

Natural corn plant exudate medium was prepared according to the following process. Corn plants were grown in pots for three weeks in a grow room and then removed from the pots. The plant roots were washed and then placed in flasks filled with 500-1000 mL of sterile water. The stems and leaves of the plants remained above the liquid level exposed to air and light. Flasks were covered in foil and placed back in the grow room for two days. The aqueous solution in the flasks was filter sterilized every 4-8 hours. After the two days, the plants were removed, and the natural plant exudate medium was filter sterilized one more time. The natural corn plant exudate media produced from two replicates was then assayed to measure amino acid concentrations. The concentration of each amino acid is shown in Tables 5 and 6 and FIGS. 3 and 4.

TABLE 5
Sample Corn Root Exudate in Water
Sample Corn Root Exudate in Water (#4)

| Amino Acid | nms/Inj | nms/50 μL | ugr/50 μL | mole % | weight % |
|---|---|---|---|---|---|
| Asp | 0.284 | 0.29 | 0.033 | 2.12 | 2.78 |
| Thr | 0.097 | 0.1 | 0.01 | 0.73 | 0.83 |
| Ser | 0.136 | 0.14 | 0.012 | 1.02 | 1.01 |
| Glu | 0.542 | 0.55 | 0.071 | 4.05 | 5.95 |
| Pro | 0.564 | 0.57 | 0.056 | 4.22 | 4.66 |
| Gly | 1.222 | 1.24 | 0.071 | 9.14 | 5.93 |
| Ala | 5.458 | 5.55 | 0.395 | 40.83 | 33 |
| Val | 0.84 | 0.85 | 0.085 | 6.28 | 7.08 |
| Ile | 0.341 | 0.35 | 0.039 | 2.55 | 3.28 |
| Leu | 0.671 | 0.63 | 0.071 | 4.62 | 5.94 |
| Tyr | 0.289 | 0.29 | 0.048 | 2.16 | 4.01 |
| Phe | 0.286 | 0.29 | 0.043 | 2.14 | 3.58 |
| His | 0.133 | 0.14 | 0.019 | 0.99 | 1.55 |
| Lys | 0.139 | 0.14 | 0.018 | 1.04 | 1.52 |
| Arg | 0 | 0 | 0 | 0 | 0 |
| Cit | 0 | 0 | 0 | 0 | 0 |
| Asn | 0 | 0 | 0 | 0 | 0 |
| Orn | 0.099 | 0.1 | 0.013 | 0.74 | 1.11 |
| Met | 0.151 | 0.15 | 0.02 | 1.13 | 1.68 |
| GABA | 2.171 | 2.21 | 0.192 | 16.24 | 16.08 |
| Trp | 0 | 0 | 0 | 0 | 0 |
| a-amino-n-buty | 0 | 0 | 0 | 0 | 0 |
| Gln | 0 | 0 | 0 | 0 | 0 |
| H-Lys | 0 | 0 | 0 | 0 | 0 |
| Total | | 13.6 | 1.2 | | 100 |
| Total μg | | | 12 | | |
| Conc. (μg/μL) | | | 0.06 | | |

TABLE 6
Sample Corn Root Exudate in Water
Sample Corn Root Exudate in Water (#5)

| Amino Acid | nms/Inj | nms/50 μL | ugr/50 μL | mole % | weight % |
|---|---|---|---|---|---|
| Asp | 0.093 | 0.1 | 0.011 | 2.91 | 3.94 |
| Thr | 0 | 0 | 0 | 0 | 0 |
| Ser | 0 | 0 | 0 | 0 | 0 |
| Glu | 0.068 | 0.07 | 0.009 | 2.13 | 3.23 |
| Pro | 0.093 | 0.1 | 0.009 | 2.91 | 3.32 |
| Gly | 0.406 | 0.42 | 0.024 | 12.71 | 8.54 |
| Ala | 1.44 | 1.5 | 0.107 | 45.07 | 37.7 |
| Val | 0.247 | 0.26 | 0.026 | 7.73 | 9.01 |
| Ile | 0.125 | 0.13 | 0.015 | 3.91 | 5.21 |
| Leu | 0.279 | 0.29 | 0.033 | 8.73 | 11.63 |
| Tyr | 0.076 | 0.08 | 0.013 | 2.38 | 4.57 |
| Phe | 0 | 0 | 0 | 0 | 0 |
| His | 0.057 | 0.06 | 0.008 | 1.78 | 2.88 |
| Lys | 0 | 0 | 0 | 0 | 0 |
| Arg | 0 | 0 | 0 | 0 | 0 |
| Cit | 0 | 0 | 0 | 0 | 0 |
| Asn | 0 | 0 | 0 | 0 | 0 |
| Orn | 0 | 0 | 0 | 0 | 0 |
| Met | 0 | 0 | 0 | 0 | 0 |
| GABA | 0.311 | 0.32 | 0.028 | 9.73 | 9.97 |
| Trp | 0 | 0 | 0 | 0 | 0 |
| a-amino-n-buty | 0 | 0 | 0 | 0 | 0 |
| Gln | 0 | 0 | 0 | 0 | 0 |
| H-Lys | 0 | 0 | 0 | 0 | 0 |
| Total | | 3.33 | 0.28 | | 100 |
| Total μg | | | 2.8 | | |
| Conc. (μg/μL) | | | 0.01 | | |

### Example 2: Microbial Growth and Nitrogenase Enzyme Activity in Plant Exudate Media

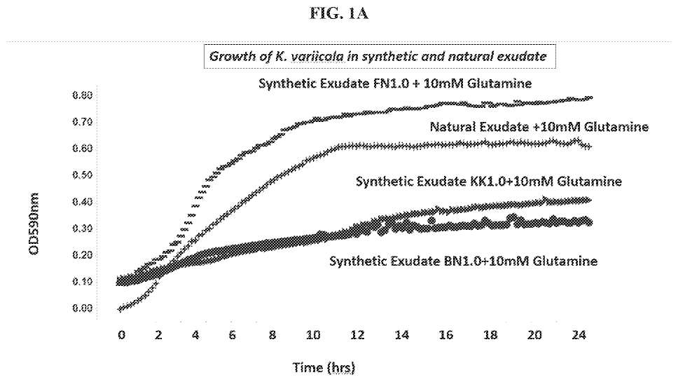

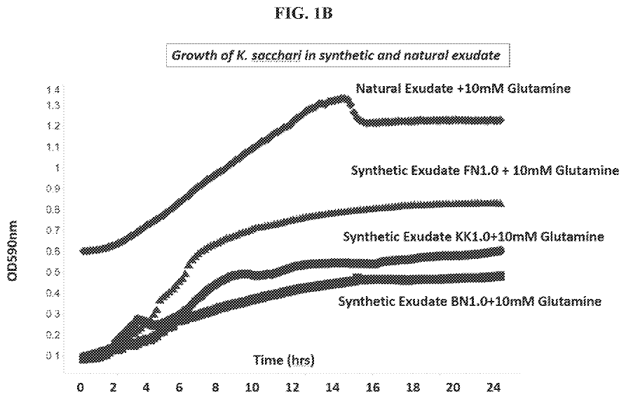

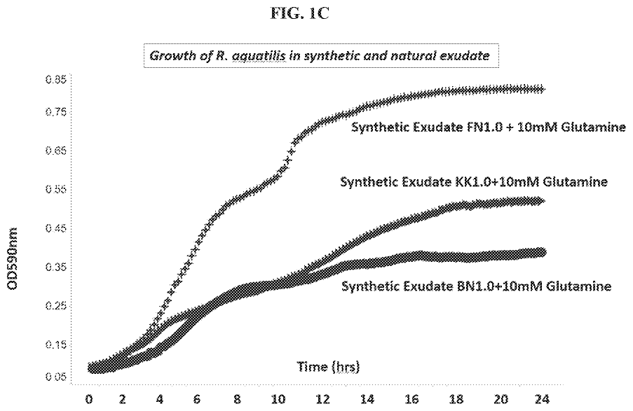

K. variicola, K. sacchari, and R. aquatilis bacteria were cultured in synthetic and natural exudate media. The natural exudate media was obtained according to the following process. Corn plants were grown in pots for three weeks in a grow room and then removed from the pots. The plant roots were washed and then placed in flasks filled with 500-1000 mL of sterile water. The stems and leaves of the plants remained above the liquid level exposed to air and light. Flasks were covered in foil and placed back in the grow room for two days. The aqueous solution in the flasks was filter sterilized every 4-8 hours. After the two days, the plants were removed, and the natural plant exudate medium was filter sterilized one more time. The synthetic exudate media was obtained according to the recipes of Tables 2-4. Both the natural and synthetic plant exudate media were supplemented with 10 mM glutamine. Growth curves for the K. variicola and K. sacchari bacteria in each of the four media are shown in FIGS. 1A and 1B, respectively. Growth curves of the R. aquatilis bacteria in each of the three synthetic media are shown in FIG. 1C. The K. variicola bacteria grew best in the synthetic exudate media of Table 4 and showed similar growth in the natural plant exudate medium.

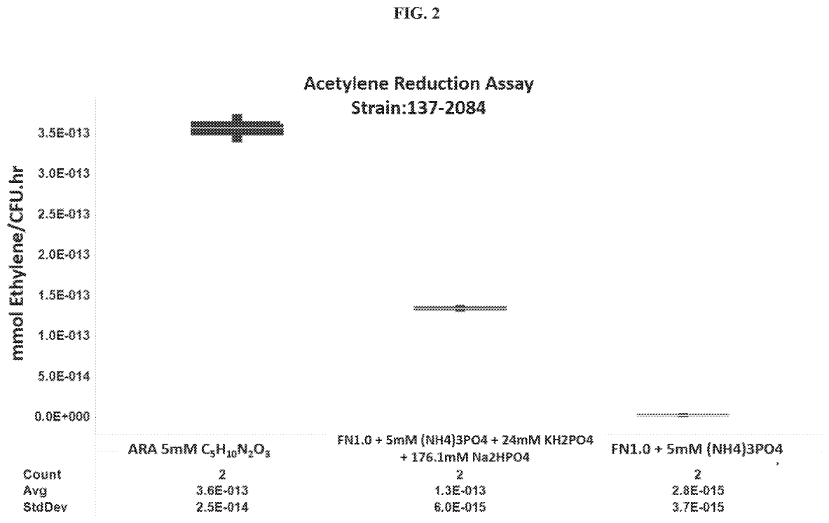

Additionally, an ARA was performed with K. variicola strain 137-2084 in control media (ARA, Table 7), in the synthetic exudate media of Table 4 supplemented with 5 mM ammonium phosphate, and in the synthetic exudate media of Table 4 supplemented with 5 mM ammonium phosphate and additional phosphate (25 g/L of Na₂HPO₄ and 3 g/L KH₂PO₄). As shown in FIG. 2, nitrogenase activity can be seen in synthetic exudate media.

TABLE 7
ARA media

20X MoFe Solution
10X ARA Sugar Buffer
1X Salt Solution
N Source (optional)
Complete ARA Media (1 L)
900 mL 1X salt solution
100 mL 10X ARA Sugar Buffer
Optional N Source: Glutamine or Ammonium
Phosphate [final = 1-10 mM as needed]
*CI137 is not repressed by glutamine. Glutamine
is generally used for the seed train and ammonium
phosphate is generally used for the assay.
Component: 20X MoFe
58 mg FeCl₃
5 mg Na₂MoO₄ 2H₂O
Dissolve in 1 L water
Filter sterilize
Component: 10X ARA Sugar Buffer
200 g sucrose
2.5 g MgSO₄ 7H₂0
10 g NaCl
CaCl₂ dihydrate (0.02 g)
Q.S. to 500 mL
Add 500 mL 20X MoFe solution
Filter sterilize
Component: 1X ARA Salt Solution
25 g Na₂HPO₄
3 g KH₂PO₄
dissolve in 900 mL water
pH to 7.5 with HCl
Autoclave or filter sterilize
Component: 200 mM Glutamine (stored at −20° C.)
or Ammonium phosphate (stored at RT)
prepped in water
Filter sterilize

### Example 3: A Lower Peak OD in Corn Root Slurry Correlates to a Lower Rate of Colonization in Corn Roots

To evaluate whether results obtained from tests carried out in root slurry or exudate correlate to results in planta, root slurry samples were generated. Experiments to determine growth metrics of different species and their derivative mutants were carried out in the slurry. Colonization experiments were also carried out to evaluate the relative colonization phenotype of those species in the corn rhizosphere.

Exudate/slurry preparation method. Two slurries of corn roots were generated: 1) greenhouse slurry or slurry generated from plants grown in sterile vermiculite for 5 weeks in a greenhouse and 2) grow room slurry, or slurry generated from plants grown in sterile sand for 5 week in a grow room. In both cases, plants were grown in the presence of nitrogen fertilizer. To capture plant slurry, corn plants were grown in sterile deep-well planter pots in either a greenhouse or a grow room with the appropriate potting medium: sterile sand in the grow room or sterile vermiculite in the greenhouse. For grow-room slurry, plants were grown in a controlled environment growth chamber under LED lighting with a 16-hour day length, maintaining 28° C. day temperatures and 22° C. night temperatures, 50-65% humidity, with daily watering and fertigation with Hoagland's containing 16 mM nitrogen every other day starting at 5 days after planting. For greenhouse slurry, plants were maintained under standard greenhouse conditions with a 15-hour day length and temperature set points of 25° C. during daylight hours and 22° C. during night hours, with watering every other day and fertigation beginning after day 7 with Hoagland's solution containing 8 mM of total nitrogen. Plants were grown to five weeks and then removed from pots and roots were washed of debris. Plants were then cut 0.5 inches below the crown and stalks were separated from the root mass. Rootballs from plants grown under the same conditions were combined and weighed out to about 500 g. An equivalent volume of water (i.e., ˜500 mL) was added to the roots and then blended until a uniform consistency was achieved. This slurry was allowed to sit at 4° C. for 24 hours. The slurry was then passed through porous cloth to remove large debris and then spun down to separate the dissolved metabolites from smaller particles. After spinning, the supernatant was passed through a 0.2 micron filter to sterilize the slurry.

Growth curve method. All strains were streaked onto super optimal broth (SOB) agar plates for single colonies from previously verified glycerol stocks. Four colonies per strain were inoculated into a 2 mL, 96-well deep-well plate containing 1 mL SOB broth. The plate was covered with a 0.2 μM breathable seal and incubated at 30° C., 200 RPM for 20-24 hrs. The next day, in a sterile 2 ml deep-well plate, 950 mL of phosphate buffered saline was added to each well of a new sterile 96-well deep-well plate. After overnight incubation, 50 μL of the overnight culture from the SOB plate was then transferred into the 1:20 dilution plate and mixed after each transfer. To set up the growth curves, 180 !IL of the assay medium/media was added to each well of a sterile 0.3 ml clear, lidded Greiner assay plate. Then, 20 μL of the 1:20 diluted culture from the 1:20 dilution plate was transferred into the Greiner assay plate in same order as the dilution plate. The assay plate was then placed into a Molecular Devices i3X plate reader. Data was captured on the i3X using the settings in Table 8 below.

TABLE 8
i3X settings for data collection

Mode: Kinetic
Temperature: 30° C.
Absorbance: 590 nm
Shaking: Medium, orbital
Read Interval: 10:59 min
Total Read Time: 24 hours
Shake time between reads: 600 sec
Shake before read: 5 sec
Plate type: Greiner Clear, Lidded
Read Order: Row

Peak OD, i.e., the highest OD measured over the course of the experiment, was noted. Where applicable, doubling times were calculated using an in-house application that employs industry standard calculations. Doubling times can also be calculated manually as follows:

$$Doubling Time ( dT ) = \frac{ln ( 2 )}{growth rate ( k )}$$

$$whereas growth rate ( k ) = \frac{[ Log ( OD start ) - Log ( OD end ) ] * 2.303}{{T}_{i} - {T}_{f}}$$

$$such that dT = \frac{ln ( 2 )}{[ Log ( OD start ) - Log ( OD end ) ] * 2.303} * \frac{1}{{T}_{i} - {T}_{f}}$$

Grow-room colonization experiment methods. At least 12 replicate plants per strain were planted in sterile sand media. At the time of planting, each plant was inoculated with 1 mL of an overnight SOB culture of a bacterial strain as specified, corresponding to ˜1×10⁹ bacterial cells, pipetted directly over the seeds. Plants were grown in a controlled environment growth chamber under LED lighting with a 16-hour day length, maintaining 28° C. day temperatures and 22° C. night temperatures, 50-65% humidity, with daily watering and fertigation every other day starting at 5 days after planting. Twenty-one days after planting, the plants were harvested, and roots were shaken clean, washed gently with sterile water, and frozen for future genomic DNA extraction. Genomic DNA extraction and colonization measurement via qPCR was carried out as described. Roots were shaken gently to remove loose particles, and root systems were separated and soaked in a RNA stabilization solution (Thermo Fisher P/N AM7021) for 30 minutes. The roots were then briefly rinsed with sterile deionized water. Samples were homogenized using bead beating with ½-inch stainless steel ball bearings in a tissue lyser (TissueLyser II, Qiagen P/N 85300) in 2 ml of lysis buffer (Qiagen P/N 79216). Genomic DNA extraction was performed with ZR-96 Quick-gDNA kit (Zymo Research P/N D3010), and RNA extraction using the RNeasy kit (Qiagen P/N 74104).

Four days after planting, 1 ml of a bacterial overnight culture (approximately 109 cfu) was applied to the soil above the planted seed. Seedlings were fertilized three times weekly with 25 ml modified Hoagland's solution supplemented with 0.5 mM ammonium nitrate. Four weeks after planting, root samples were collected and the total genomic DNA (gDNA) was extracted. Root colonization was quantified using qPCR with primers designed to amplify unique regions of either the wild type or derivative strain genome. QPCR reaction efficiency was measured using a standard curve generated from a known quantity of gDNA from the target genome. Data was normalized to genome copies per g fresh weight using the tissue weight and extraction volume. For each experiment, the colonization numbers were compared to untreated control seedlings.

### Results:

FIGS. 5A and 5B show growth and colonization measurements for Kosakonia sacchari 6 and its derivatives 6-2425 and 6-2634. FIG. 5A shows a boxplot of at least three replicate peak OD measurements of the strains in the greenhouse slurry. FIG. 5B shows the colonization in cells per gram of fresh weight root tissue (CFU/g FW) of at least 10 replicate corn plants inoculated with the specified strain. Both derivative strains, 6-2425 and 6-2634, show a decrease in both peak OD and colonization compared to the wild-type 6 strain.

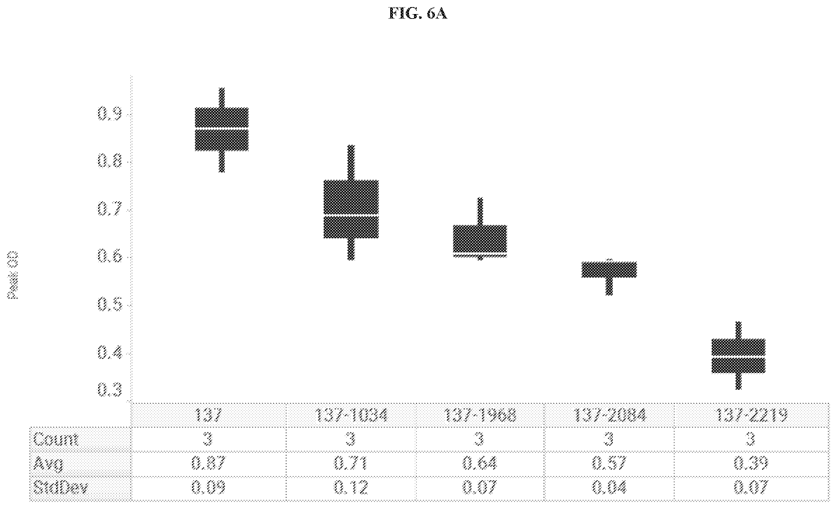

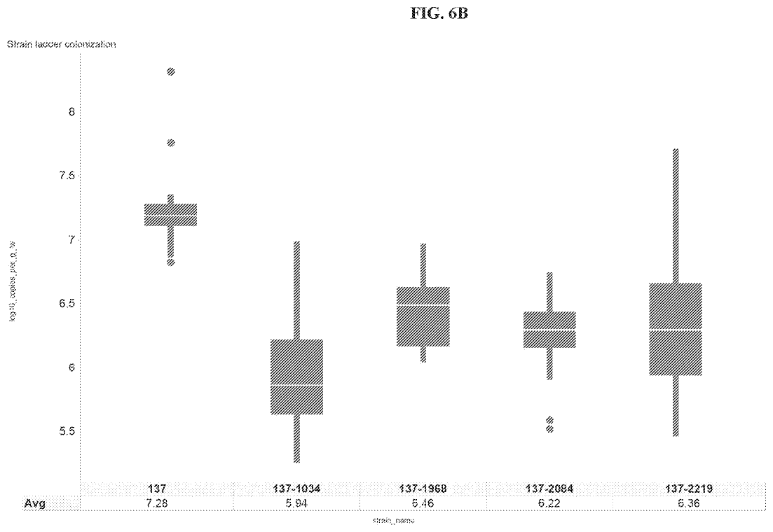

FIGS. 6A and 6B show growth and colonization measurements for Klebsiella variicola 137 and its derivatives 137-1034, 137-1968, 137-2084, and 137-2219. FIG. 6A shows a boxplot of at least three replicate peak OD measurements of the strains in the greenhouse slurry. FIG. 6B shows the colonization in CFU/g FW of at least 10 replicate corn plants inoculated with the specified strain. All derivative strains show a decrease in both peak OD and colonization compared to the wild-type 137 strain.

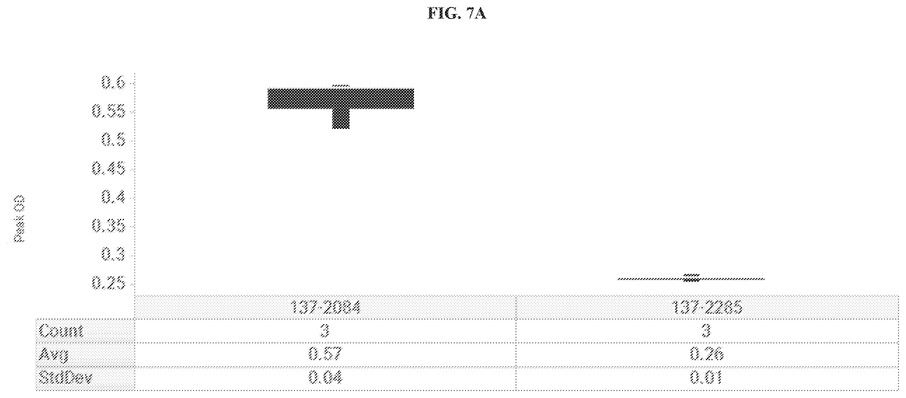

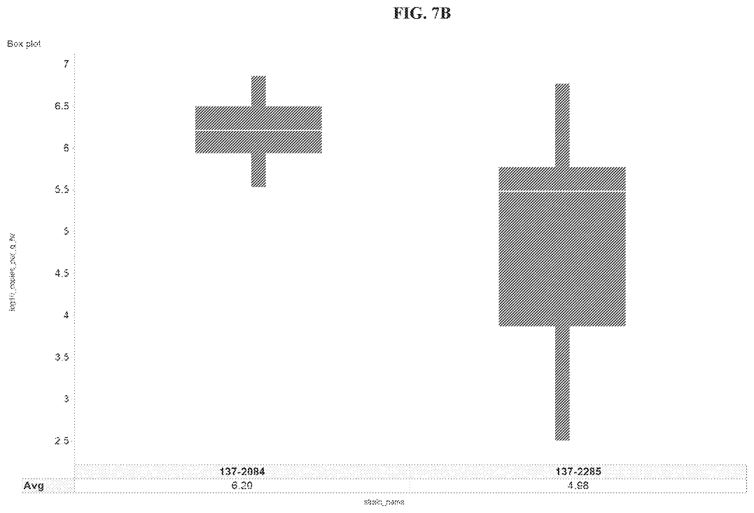

FIGS. 7A and 7B show growth and colonization measurements for two K. variicola 137 mutant strains. Strain 137-2285 is a mutant derivative of strain 137-2084. FIG. 7A shows a boxplot of at least three replicate peak OD measurements of the strains in the greenhouse slurry. FIG. 7B shows the colonization in CFU/g FW of at least 10 replicate plants inoculated with the specified strain. In both peak OD in corn root slurry and colonization of corn roots, 137-2285 shows a decreased fitness compared to its parent strain 137-2084.

The results in FIGS. 5A-7B show how measuring peak OD in corn root slurry can allow for the selection of strains with altered corn root colonization phenotype, e.g., decreased colonization capability, in two distinct species of diazotrophic bacteria.

### Example 4: A Slower Log-Phase Growth Rate (Doubling Time) in Corn Root Slurry Correlates to a Lower Rate of Colonization in Corn Roots

Using the same slurries and methods described in Example 1, diazotrophic strains were further analyzed for doubling time in corn root slurries and colonization of corn roots.

### Results:

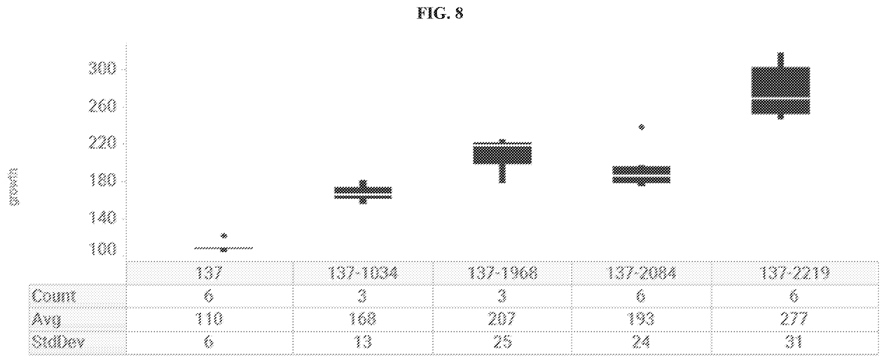

FIG. 8 shows growth measurements for Klebsiella variicola 137 and its derivatives 137-1034, 137-1968, 137-2084, and 137-2219, specifically, a boxplot of the doubling time from at least three replicate growth curves of the strains in grow room corn root slurry. All derivative strains show both an increase in doubling time (i.e., a decreased growth rate) and a decreased colonization in corn roots (FIG. 6B) compared to the wild-type 137 strain.

The results in FIG. 8 show how measuring growth rate (doubling time) in corn root slurry can allow for the selection of strains with altered corn root colonization phenotype, e.g., decreased colonization capability, in two distinct species of diazotrophic bacteria.

### Example 5: More Robust Growth in Corn Root Slurry Corresponds to a Higher Rate of Colonization of Corn Roots Compared to WT and Other Strains

Using the same slurries and methods described in Example 3, diazotrophic strains were further analyzed for growth in corn root slurries and colonization of corn roots. In this example, peak OD was measured in greenhouse slurry as described in Example 3. Additionally, growth in greenhouse slurry was measured in a BioLector micro-fermenter format.

BioLector growth assay methods. All strains were streaked onto SOB agar plates for single colonies from previously verified glycerol stocks. A single colony per strain was inoculated into 5 mL SOB broth incubated at 30° C., 200 RPM for 20 hours. The greenhouse corn root slurry was inoculated with 2% inoculum and 1 ml was transferred to the 48-well FlowerPlate and grown aerobically for 24 hours at 30° C. in a BioLector (The Microbioreactor Company) micro-fermenter system. BioLector settings were as described in Table 9 below.

TABLE 9
BioLector settings

| | |
|---|---|
| Temperature | 30° C. |
| Humidity | 85% |
| Shaker | 1000 rpm |
| CycleTime | 15 min |
| Fill Volume | 1000 μl |
| Microtiter plate | MTP-48-BOH2 Batch Plate (FlowerPlate) |
| | with pH- and DO-optodes |
| Sealing foil | F-GPR48-10 (Gas-permeable sealing foil, |
| | reduced evaporation) |
| Parameters measured | Biomass gain 4, pH, dissolved oxygen (DO) |

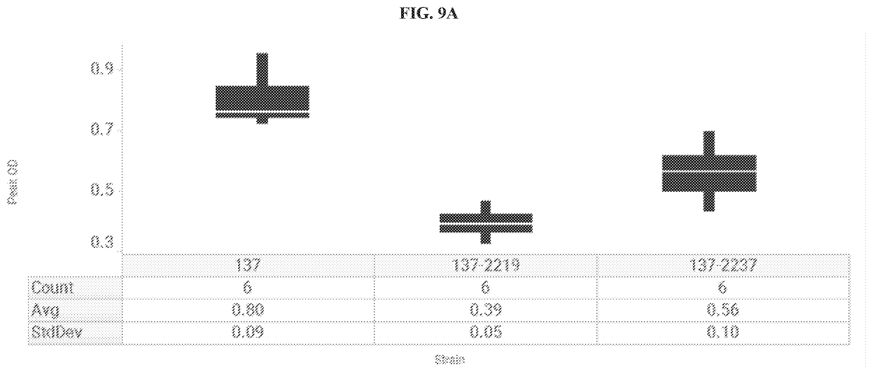

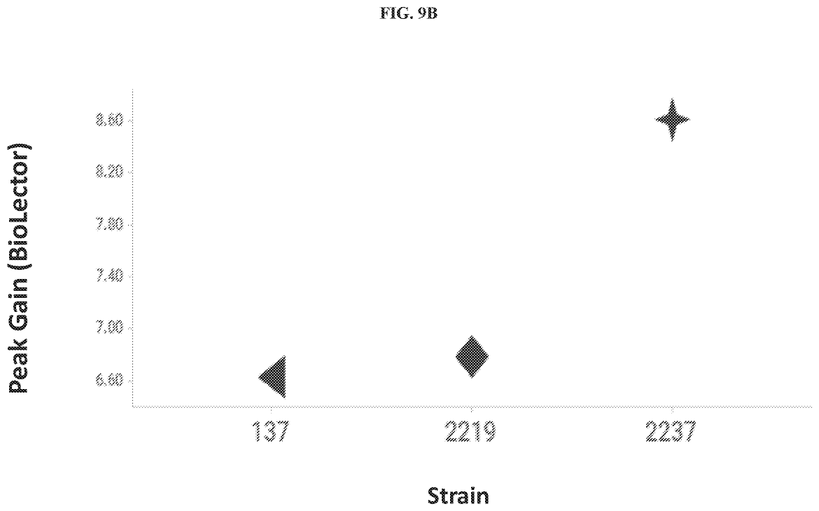

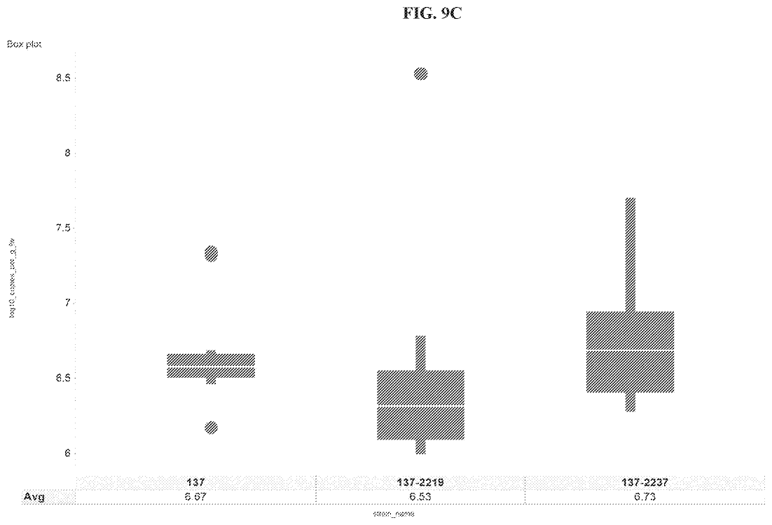

FIGS. 9A-9C show growth and colonization measurements for K. variicola 137 and two of its derivative mutants, 137-2219 and 137-2237. FIG. 9A shows the average at least six replicate peak OD measurements of the strains in the greenhouse slurry. FIG. 9B shows the average peak gain in greenhouse slurry in a BioLector growth experiment. In both experimental formats, 137-2237 shows more robust growth than 137-2219. FIG. 9C shows the colonization in CFU/g FW of at least 10 replicate plants inoculated with the specified strain. In this example, the more robust growth of 137-2237 in corn root slurry compared to 137-2219 corresponded to a more robust colonization capability in corn roots.

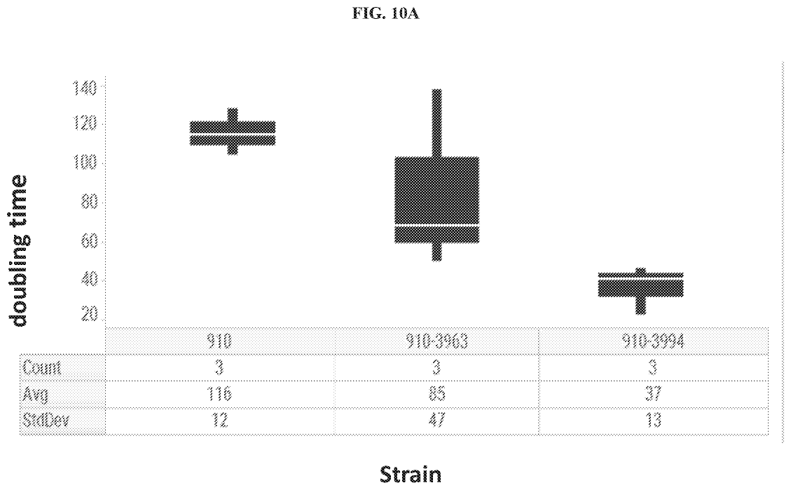

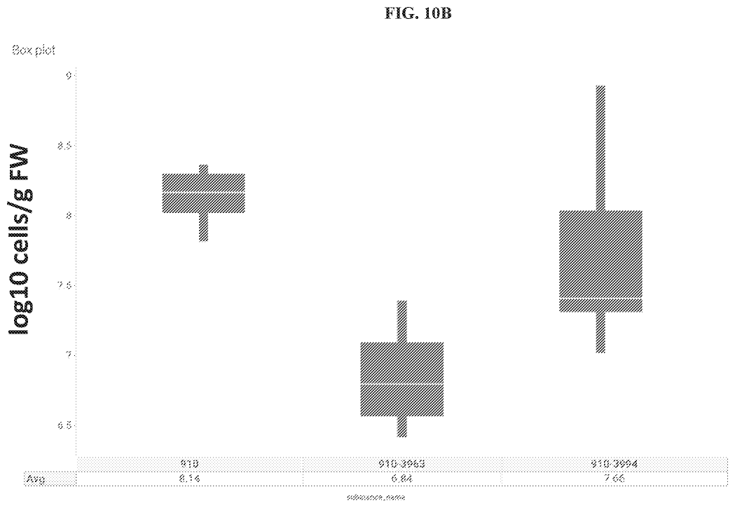

FIGS. 10A and 10B show growth and colonization measurements for Metakosakonia intenstini 910 and two of its derivative mutants, 910-3994 and 910-3963. FIG. 10A shows a boxplot of doubling time from at least three replicate growth curves of the strains in grow-room corn root slurry. FIG. 10B shows the colonization in CFU/g FW of at least 10 replicate plants inoculated with the specified strain. In this example, the more robust growth of 910-3994 in corn root slurry compared to 910-3963 corresponded to a more robust colonization capability in corn roots.

The results in FIGS. 9A-10B show how growth metrics in corn root slurry can allow for the selection of strains with altered corn root colonization phenotype, e.g., increased colonization capability, in two distinct species of diazotrophic bacteria.

### Example 6: Higher Growth in Slurry Corresponds to Improvement in Corn Fresh Weight

Using the same slurries and methods described in Example 3, diazotrophic strains were further analyzed for doubling time in corn root slurries. Then a greenhouse experiment was carried out to assess the early vegetative growth of corn plants inoculated with select strains.

Corn growth assay methods. A corn growth assay was performed under standard greenhouse conditions with a 15-hour day length and temperature set points of 25° C. during daylight hours and 22° C. during night hours. Seeds were planted in standard potting mix combined 1:1 with calcined clay by pressing two 2-inch holes near the center of each pot with a planting tool. One seed was then dropped into each prepared hole and inoculated with sterile PBS (UTC controls) or an equal volume of bacterial suspension using cells diluted to a set optical density. For positive control plants, 1.8 g of Nitroform controlled release urea was amended to the top 1-2 inches of each pot at the time of planting. Otherwise, these plants were treated the same as the negative UTC control with PBS and no microbe inoculated at planting. Seedlings were given only water for the first week, then thinned to a single plant per pot by selecting the most vigorous seedling and removing the remaining plant at approximately 7 days after planting. At one-week post planting, fertigation began on all plants using a modified Hoagland's solution containing 8 mM of total nitrogen. Fertigation typically occurred twice per week, and additional water was given to all plants as needed throughout the duration of the experiment. Shoot fresh weight was measured immediately after harvest by cutting the stalk at the soil surface and immediately weighing the above-ground portion of plant tissue.

### Results:

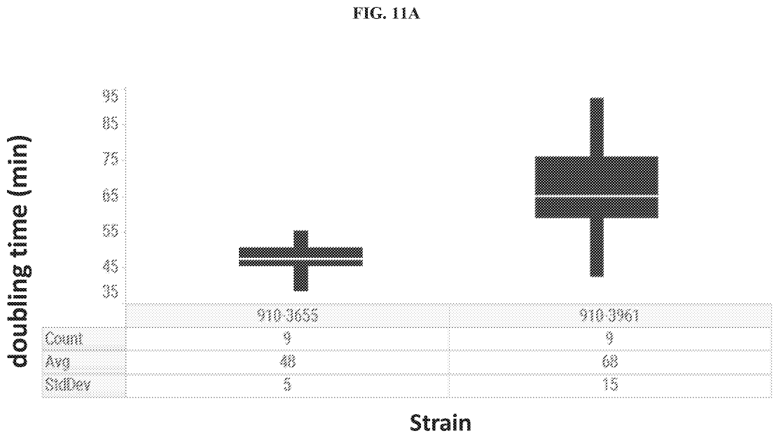

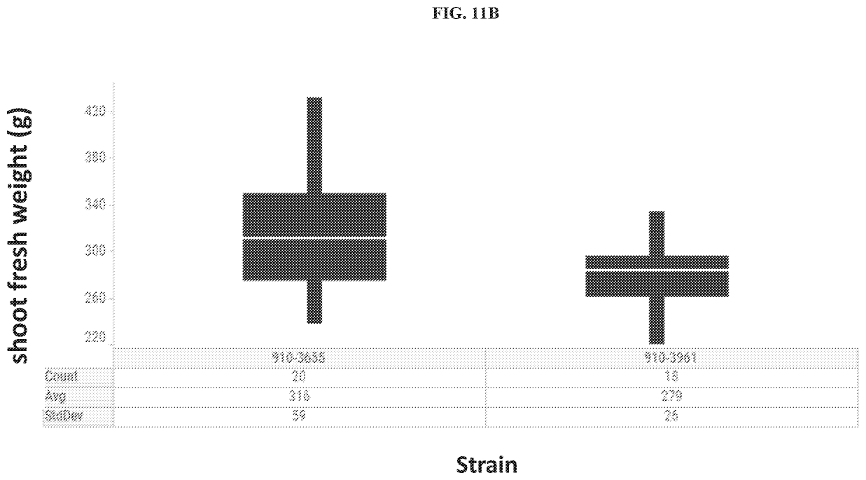

FIGS. 11A and 11B show growth in root slurry of M intenstini 910 strains 910-3655 and 910-3961, as well as biomass of corn plants inoculated with each strain. 910-3655 exhibited a faster doubling time (i.e., higher growth rate) in grow room and corn root slurry (FIG. 11A). In a corn growth assay, plants inoculated with 910-3655 exhibited higher fresh weight than plants inoculated with 910-3961 (FIG. 11B). These results show that growth in corn root slurry can be used to select a strain with a larger impact on corn growth.

### Example 7: Growth in Root Exudate/Slurry can be Used to Select Species with High Root Colonization in Greenhouse and Field

Using the same slurries and methods described in Example 3, multiple species were assayed for differences in growth in root exudate and differences in corn root colonization. Growth chamber colonization assays were carried as described in Example 3. Additionally, a multi-location field trial was carried out to evaluate colonization of the strains in relevant agricultural conditions.

Colonization field trial methods. Field trials to test microbial colonization of corn roots were carried out in ten locations across nine states: Texas, Georgia, California, South Carolina, Louisiana, Missouri, Nebraska, Ohio, and Illinois. Trials consisted of randomized complete block design with four reps at 66% of recommended N fertilizer (determined locally for each location), and sites were planted between March and June 2019. Microbial treatments consisting of concentrated cultures were applied to seeds and the treated seeds were held at 4° C. until planting. Root samples were collected from three representative plants from each plot at two time points, V4-V6 and V10-V12. Roots were immediately packaged on ice and shipped overnight for processing the following day using the same procedures applied to greenhouse plant samples. On arrival, roots were washed free of soil and a 1-inch section of seminal and node 1 roots directly below the seed was homogenized and total genomic DNA (plant and microbial) was extracted. Colonization was measured using microbe-specific qPCR normalized to input tissue fresh weight and compared to untreated control plants as described in WO 2019/032926 A1, paragraphs 303 and 304.

### Results:

FIGS. 12A-12D show both growth in corn root slurry and colonization of three diazotrophic species: Paraburkholderia tropica 8, Paenibacillus polymixa 41, and Herbaspirillum aquaticum 3069. Growth data is shown for at least three replicates per strain. In corn root slurry, strain P. tropica 8 exhibited both the highest peak OD (FIG. 12A), and strain H. aquaticum 3069 exhibited the fastest doubling time (FIG. 12B), while strain P. polymixa 41 showed the lowest peak OD (FIG. 12A) and slowest doubling time (FIG. 12B). FIGS. 12C and 12D show the colonization of these strains in field trials at two different locations, showing high colonization of corn roots by both P. tropica 8 and H. aquaticum 3069 compared to P. polymixa 41. These results show that growth in corn root slurry can be used to select strains with superior corn root colonization phenotypes from a set of diverse species.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of predicting an in planta phenotype of a microbial strain, the method comprising:
(a) culturing a microbial strain in a plant exudate medium (PEM);
(b) assaying an in vitro phenotype of said microbial strain; and
(c) using said in vitro phenotype from (b) to predict an in planta phenotype of said microbial strain.

2. method of claim 1, wherein said microbial strain is isolated from a soil sample.

3. The method of claim 1, wherein said microbial strain is a genetically modified microbial strain.

4.-32. (canceled)

33. A method of selecting a genetically modified microbial strain having an altered in planta phenotype, the method comprising:
(a) culturing a genetically modified microbial strain in a plant exudate medium (PEM);
(b) assaying an in vitro phenotype of said genetically modified microbial strain; and
(c) selecting said genetically modified microbial strain if it exhibits an alteration in said in vitro phenotype compared to a non-genetically modified microbial strain of the same species cultured under similar conditions, thereby selecting said genetically modified microbial strain having said altered in planta phenotype.

34.-38. (canceled)

39. The method of claim 33, wherein said in vitro phenotype is nitrogen fixation activity, ammonium excretion, growth rate, peak optical density of a microbial strain culture, or growth in a cell growth competition assay.

40. (canceled)

41. (canceled)

42. The method of claim 33, wherein said in planta phenotype is promotion of plant growth, plant colonization ability, or rhizosphere fitness.

43.-60. (canceled)

61. The method of claim 33, wherein said genetically modified microbial strain is produced by random mutagenesis, site-directed mutagenesis, or transposon mutagenesis.

62. (canceled)

63. (canceled)

64. The method of claim 33, wherein said genetically modified microbial strain is an endophyte, an epiphyte, or rhizospheric.

65.-70. (canceled)

71. A method of selecting a plant-associated microbe that is attracted to a component of a plant exudate medium (PEM), the method comprising:
(a) obtaining or providing a semisolid agar plate comprising multiple regions, said multiple regions comprising:
(i) a first region comprising agar dissolved in a rich medium;
(ii) a last region comprising agar dissolved in said PEM; and
(iii) a plurality of intermediate regions that each comprise a mix of said rich medium and said PEM to form a gradient from said first region to said last region;
(b) applying a plurality of putative plant-associated microbes to said first region;
(c) culturing said plurality of putative plant-associated microbes for a period of time; and
(d) collecting one or more microbes which have migrated the furthest from said first region toward said last region, thereby selecting said plant-associated microbe.

72. The method of claim 71, further comprising:
(e) selecting a plurality of collected plant-associated microbes from step (d);
(f) obtaining an additional semisolid agar plate as described in (a);
(g) applying the plurality of collected plant-associated microbes from step (e) to said additional semisolid agar plate; and
(h) collecting one or more microbes which have migrated the furthest from said first region toward said last region, wherein said collected microbes optionally are exposed to a mutagen prior to performing steps (e) to (h).

73.-78. (canceled)

79. The method of claim 71, wherein said plurality of putative plant-associated microbes comprises wildtype strains, genetically modified microbes, microbes isolated from an environmental sample, a library of strains formed by mutagenesis, or one or more strains with defects in DNA repair.

80.-94. (canceled)

95. A method of conducting a field trial of a plant beneficial microbial strain, comprising:
(a) culturing a plurality of plant beneficial microbial strains in a plant exudate medium (PEM);
(b) assaying an in vitro phenotype of said plurality of plant beneficial microbial strains;
(c) selecting a plant beneficial microbial strain that exhibits a desired in vitro phenotype;
(d) contacting said selected plant beneficial microbial strain with plants in a field; and
(e) assessing a plant phenotype of said plants in said field as compared to similar plants in a similar field which are not contacted with said selected plant beneficial microbial strain.

96. The method of claim 95, wherein said plurality of plant beneficial microbial strains comprises a plurality of different species of microbes or a plurality of genetic variants of a single microbial species.

97. (canceled)

98. The method of claim 95, further comprising selecting a plant beneficial microbial strain in step (c) when the desired in vitro phenotype is high titer growth in PEM or rapid growth rate in PEM.

99.-140. (canceled)

141. An engineered microbe which comprises a modification which alters chemoattraction to a component of a plant exudate medium (PEM), optionally a natural PEM (NPEM).

142. The engineered microbe of claim 141, wherein said altered chemoattraction is improved chemoattraction.

143. The engineered microbe of claim 141, wherein said altered chemoattraction is decreased chemoattraction.

144. The engineered microbe of claim 141, wherein said engineered microbe is a diazotrophic bacterium or a phosphate-solubilizing bacterium.

145. (canceled)

146. (canceled)

147. The engineered microbe of claim 141, wherein said NPEM is formed by steeping a root system of a plant in an aqueous solution, an aeroponic system, semisolid agar, an absorbent surface, or an adsorbent surface or said NPEP is formed by homogenizing a plant part or a plant root system in an aqueous solution.

148.-153. (canceled)

154. The engineered microbe of claim 147, wherein said plant is a cereal plant.

155. The engineered microbe of claim 147, wherein said plant is selected from the group consisting of: corn, soybean, canola, sorghum, potato, rice, barley, fonio, oats, Palmer's grass, rye, pearl millet, sorghum, spelt, teff, triticale, wheat, breadnut, buckwheat, cattail, chia, flax, grain amaranth, hanza, quinoa, and sesame.

156. (canceled)

157. (canceled)
